(19) United States
(12) Patent Application Publication
(10) Pub. No.: US 2026/0151509 A1
(43) Pub. Date: Jun. 4, 2026

(54) COMPOSITIONS AND METHODS FOR PREVENTING, AMELIORATING, OR TREATING SICKLE CELL DISEASE

(71) Applicants: INCISIVE GENETICS INC., Vancouver (CA); THE UNIVERSITY OF BRITISH COLUMBIA, Vancouver (CA)

(72) Inventors: Blair LEAVITT, Vancouver (CA); Austin HILL, Vancouver (CA); Pamela WAGNER, Vancouver (CA); Nicholas CARON, Vancouver (CA)

(21) Appl. No.: 18/706,761

(22) PCT Filed: Nov. 2, 2022
(86) PCT No.: PCT/IB2022/060572

§ 371 Date: May 2, 2024

Related U.S. Application Data
(60) Provisional application No. 63/274,630, filed on Nov. 2, 2021.

(51) Int. Cl. A61K 48/00; A61P 7/00; C12N 9/22; C12N 15/11

(52) U.S. Cl. CPC A61K 48/0058; A61K 48/0033; A61K 48/0041; A61P 7/00; C12N 9/226; C12N 15/11; C12N 2310/20

(57) ABSTRACT

The present disclosure provides nucleic acids, compositions and vectors containing and their use for effecting gene editing and/or gene expression alteration on sickle cell disease (SCD)-associated genes invivo. The present disclosure further provides methods of effecting gene editing and/or gene expression alteration on SCD-associated genes invivo and methods of preventing, ameliorating, or treating SCD.

### RELATED APPLICATIONS

The present disclosure claims benefit of priority to, and incorporates by reference the contents of U.S. Provisional Application No. 63/274,630, filed on Nov. 2, 2021.

### SEQUENCE LISTING

The contents of the electronic sequence listing (2957415.001601.xml; Size: 154,443 bytes; and Date of Creation: Oct. 26, 2022) is herein incorporated by reference in its entirety.

### FIELD OF THE INVENTION

The present disclosure relates to compositions and methods for preventing, ameliorating, and/or treating Sickle cell disease (SCD). The present disclosure also relates to compositions and methods for effecting gene editing and/or gene expression alteration in vivo using a lipid-based, transfection competent vesicle (TCV) in cells in the bone marrow or of bone marrow origin.

### BACKGROUND OF THE INVENTION

SCD is a group of disorders characterized by a mutation(s) in and/or altered expression of HBB, the gene encoding beta-globin, which is hemoglobin (Hb)'s beta subunit (Kato et al.; Nat Rev Dis Primers, 2018 Mar. 15; 4:18010.). SCD patients carry at least one sickle Hb (HbS) allele of HBB, the βS allele, containing an adenine-to-thymine substitution relative to the wildtype HBB gene and encodes the sickle Hb (HbS) variant of beta-globin, containing a glutamate-to-valine (“E-to-V” or “E6V”) substitution.

Hb expressed during a fetus stage (HbF) is a tetramer of two alpha-globin subunits and two gamma-globin subunits and does not involve beta-globin (Philpsen, Haematologica. 2014 November; 99(11):1647-9.). SCD patients typically have no gene alterations in the gene encoding alpha- or gamma-globin and thus are not affected by the βS allele during the fetus stage. In contrast, hemoglobin that starts increasing its expression post birth (HbA) is a tetramer formed by two alpha-globin subunits and two beta-globin subunits. HbA in SCD patients (HbS) thus contains the beta-globin HbS variant, and deoxygenated HbS can polymerize and HbS polymers can stiffen the erythrocyte, causing an anemic phenotype as HbS starts to dominate. Common complications include acute pain events, acute chest syndrome and stroke, and chronic complications including chronic kidney disease can damage all organs.

Medications available for reduce the frequency of pain crisis, include hydroxyurea, L-glutamine oral powder, and crizanlizumab. General pain medications are also used to alleviate pain. A recently approved voxelotor, a HbS polymerization inhibitor, decreases sickling of HbS and extends the half-life of RBCs Hydroxycarbamide, blood transfusions, and hematopoietic stem cell transplantation can reduce the severity of the disease, but currently there is no sufficiently effective or feasible treatment or cure.

### SUMMARY OF THE INVENTION

In one aspect, the present disclosure provides one or more guide RNAs (gRNAs) for Clustered Regularly Interspaced Short Palindromic Repeats (CRISPR)-mediated gene editing and compositions containing. The gRNA may comprise at least one CRISPR RNA (crRNA) sequence comprising a target-complementary sequence comprising at least 17 nucleic acids, optionally comprising 17, 18, 19, 20, 21, 22, 23, 24, 25, 26, 27, 28, 29, or 30 nucleic acids.

In some embodiments, the target-complementary sequence may comprising: (i) the polynucleotide sequence of SEQ ID NO: 85, 25, 45, 47, 49, 65, 67, 69, 75, or 77; or (ii) a polynucleotide sequence comprising one or more (optionally one, two, three, four, or five) mutations relative to the polynucleotide sequence of SEQ ID NO: 85, 25, 45, 47, 49, 65, 67, 69, 75, or 77. In certain embodiments, the mutations may be at any nucleic acid position(s) other than the 4th to the 7th nucleic acid positions from the 3′-end of the polynucleotide sequence of SEQ ID NO: 85, 25, 45, 47, 49, 65, 67, 69, 75, or 77, respectively.

In some embodiments, the gRNA may be a single guide RNA (sgRNA) comprising (i) a crRNA sequence comprising the target-complementary sequence and a crRNA backbone sequence and (ii) a trans-activating CRISPR RNA (tracrRNA) sequence in a single strand. In certain embodiments, the crRNA sequence and the tracrRNA sequence may be linked via a linker optionally comprising SEQ ID NO: 139. In certain embodiments, the gRNA may comprise the target-complementary sequence followed by a sgRNA backbone sequence of any of SEQ ID NOS: 141-144, In certain embodiments, the sgRNA backbone sequence may be followed by one or more uracils, further optionally 1-10 uracils.

In some embodiments, the gRNA may be a dual guide RNA (dgRNA) formed by hybridization between (i) a crRNA sequence comprising the target-complementary sequence and a crRNA backbone sequence and (ii) a tracrRNA. In certain embodiments, the crRNA backbone sequence and the tracrRNA may comprise SEQ ID NOS: 145 and 146, respectively, or SEQ ID NOS: 147 and 148, respectively.

In some embodiments, the one or more gRNAs may be synthetic or recombinant.

In some embodiments, the one or more gRNAs may be a synthetic sgRNA and may comprise at least one chemical modification. In certain embodiments, the at least one chemical modification may comprise (i) 2′-O-methylation optionally at first three and last three bases and/or (ii) one or more 3′ phosphorothioate bonds, optionally between first three and last two bases.

In some embodiments, the composition may comprise any one or more of the gRNAs described above.

In another aspect, the present disclosure provides a polynucleotide or polynucleotides encoding any one or more of the isolated gRNAs described herein and compositions containing.

In some embodiments, the composition may comprise any one or more of such polynucleotides.

In another aspect, the present disclosure provides a vector comprising a polynucleotide or polynucleotides encoding any of the isolated gRNAs described herein and compositions containing.

In some embodiments, the polynucleotide or polynucleotides may optionally be linked to one or more regulatory sequences.

In some embodiments, the composition may comprise any one or more of such vectors.

In another aspect, the present disclosure provides ribonucleoproteins (RNPs), comprising: (a) any one or more isolated gRNAs described herein; which is complexed with (b) a Cas endonuclease.

In some embodiments, the Cas endonuclease may be: (i) selected from the group consisting of Cas9, Cas3, Cas8a2, Cas8b, Cas8c, Cas10, Cas11, Cas12, Cas12a or Cpf1, Cas13, Cas13a, C2c1, C2c3, and C2c2. In some embodiments, the Cas endonuclease may be a class 2 Cas endonuclease, optionally a type II, type V, or type VI Cas nuclease. In some embodiments, the Cas endonuclease may be Cas9 of Streptococcus pyogenes (SpCas9), Staphylococcus aureus Cas9 (SaCas9), Streptococcus thermophilus (StCas9), Neisseria meningitidis (NmCas9), Francisella novicida (FnCas9), Campylobacter jejuni (CjCas9), Streptococcus canis (ScCas9), Staphylococcus auricularis (SauriCas9), or any engineered variants thereof, including SaCas9-HF, SpCas9-HF1, KKHSaCas9, eSpCas9, HypaCas9, FokI-Fused dCas9, xCas9, SpRY (variant of SpCas9), and SpG (variant of SpCas9). In some embodiments, the Cas endonuclease may be Cas9. In certain embodiments, the Cas9 may comprise any one of SEQ ID NOS: 150-161.

In some embodiments, the RNP may be formed by mixing at an approximately equimolar ratio (I) a solution comprising the one or more isolated gRNAs and (II) a solution comprising the Cas endonuclease. In certain embodiments, the pH of the solution comprising the one or more isolated gRNAs may be about 6 to 8, about 6.5 to 7.5, optionally about 7. In certain embodiments, the pH of the solution comprising the Cas endonuclease may be about 6 to 8, about 6.5 to 7.5, optionally about 7. In certain embodiments, the mixing may be for about 5 minutes.

In one aspect, the present disclosure provides a pharmaceutical composition for effecting gene editing and/or gene expression alteration. In some embodiments, the gene editing and/or gene expression alteration may be effected in vivo.

In some embodiments, the pharmaceutical composition may comprise at least one cargo encapsulated in a carrier. In certain embodiments, the carrier may be a lipid-based, transfection competent vesicle (TCV). In some embodiments, the at least one cargo may be capable of effecting gene editing of at least one Sickle cell disease (SCD)-associated gene and/or a promoter or enhancer thereof in vivo in a subject in need thereof. In some embodiments, the at least one cargo may be capable of altering the expression, function, and/or effect of at least one SCD-associated gene in vivo in a subject in need thereof. In some embodiments, the at least one cargo may be capable of effecting gene editing of at least one Sickle cell disease (SCD)-associated gene and/or a promoter or enhancer thereof and altering the expression, function, and/or effect of at least one SCD-associated gene in vivo in a subject in need thereof. In some embodiments, the subject may have or may have a risk of developing SCD, which is optionally sickle cell anemia (SCA), Sickle cell-hemoglobin C (HbSC), or HbS β-thalassaemia.

In some embodiments, the pharmaceutical composition may be for: (I) direct injection into the bone marrow of the subject; and/or (II) intravenous injection into the subject who may optionally be administered at least one agent that promotes stem cell mobilization.

In some embodiments, the carrier may be a lipid-based TCV, and the TCV in the composition may comprise at least one ionizable cationic lipid. In some embodiments, the at least one ionizable cationic lipid may comprise, essentially consist of, or consist of a lipid selected from the group consisting of N,N-dimethyl-2,3-dioleyloxy)propylamine (DODMA), 1,2-dioleoyl-3-dimethylammonium propane (“DODAP”), 1,2-Dilinoleoyl-3-dimethylaminopropane (DLinDAP), N,N-dimethyl-2,2-di-(9Z,12Z)-9,12-octadecadien-1-yl-1,3-dioxolane-4-ethanamine (KC2), (6Z,9Z,28Z,31Z)-heptatriaconta-6,9,28,31-tetraen-19-yl 4-(dimethylamino)butanoate (MC3), N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxyl)propyl)-N,N,N-trimethylammonium chloride (DOTMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA·Cl), 1,2-Dilinoleoyl-3-trimethylaminopropane chloride salt (DLin-TAR·Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLin-K-DMA), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyl)tetrahydro-3 aH-cyclopenta[d][1,3]dioxol-5-amine (ALNY-100), N-(2,3-dioleyloxyl)propyl-N,N—N-triethylammonium chloride (“DOTMA”); 1,2-Dioleyloxy-3-trimethylaminopropane chloride salt (“DOTAP·Cl”); 3.beta.-(N—(N′,N′-dimethylaminoethane)-carbamoyl)cholesterol (“DC-Chol”), N-(1-(2,3-dioleyloxyl)propyl)-N-2-(sperminecarboxamido)ethyl)-N,N-dimethyl-ammonium trifluoracetate (“DOSPA”), dioctadecylamidoglycyl carboxyspermine (“DOGS”), and N-(1,2-dimyristyloxyprop-3-yl)-N,N-dimethyl-N-hydroxyethyl ammonium bromide (“DMRIE”), and any combinations thereof;

In some embodiments, the TCV may further comprise at least one helper lipid. In some embodiments, the helper lipid may comprise, essentially consist of, or consist of a lipid selected from the group consisting of dioleoylphosphatidylethanolamine (DOPE), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), and any combinations thereof;

In some embodiments, the TCV may further comprise at least one phospholipid. In some embodiments, the phospholipid may comprise, essentially consist of, or consist of a group selected from the group consisting of distearoylphosphatidylcholine (DSPC), dioleoyl phosphatidylethanolamine (DOPE), dipalmitoylphosphatidylcholine (DPPC), phosphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dilinoleoylphosphatidylcholine distearoylphophatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine, and any combinations thereof;

In some embodiments, the TCV may further comprise at least one cholesterol or cholesterol derivative. In some embodiments, the cholesterol or cholesterol derivative may comprise, essentially consist of, or consist of a cholesterol or cholesterol derivative selected from the group consisting of cholesterol, N,N-dimethyl-N-ethylcarboxamidocholesterol (DC-Chol), 1,4-bis(3-N-oleylamino-propyl)piperazine, imidazole cholesterol ester (ICE), and any combinations thereof.

In some embodiments, the TCV may further comprise at least one PEG-lipid. In some embodiments, the PEG-lipid may comprise, essentially consist of, or consist of a PEG-lipid selected from the group consisting of PEG-myristoyl diglyceride (PEG-DMG) (e.g., 1,2-dimyristoyl-rac-glycero-3-methoxypolyethylene glycol-2000 (Avanti® Polar Lipids (Birmingham, AL)), which is a mixture of 1,2-DMG PEG2000 and 1,3-DMG PEG2000 (e.g., in about 97:3 ratio)), PEG-phosphatidylethanolamine and phosphatidic acid, PEG-ceramide conjugates (e.g., PEG-CerC14 or PEG-CerC20), PEG-modified dialkylamines, PEG-modified 1,2-diacyloxypropan-3-amines, and any combinations thereof.

In some embodiment, the TCV comprising the at least one ionizable cationic lipid as described above may further comprises one or more of: the at least one helper lipid as described above; the at least one phospholipid as described above; the at least one cholesterol or cholesterol derivative as described above; and/or the at least one PEG-lipid as described above.

In some embodiments, the TCV is substantially, essentially, or entirely free of destabilizing agents.

In some embodiments, the TCV may be formed by: (a) generating a first solution by dissolving all components of the TCV, optionally at about 20-35 mM, in ethanol; (b) providing a second solution, which is aqueous and contains sodium acetate and/or sodium citrate, optionally at about 25 mM, optionally wherein the pH of the solution is about 4; (c) combining the first and second solutions by gentle mixing (optionally repeated manual reciprocation of the TCV-generating fluid in a pipette), micromixing optionally using a staggered herringbone micromixer (SHM) or T-junction or Y-junction mixing, or extrusion; and (d) removing ethanol, optionally by dialysis or evaporation.

In some embodiments, the size of the TCV before encapsulation of the at least one cargo may be in a range of about 9 nm to about 80 nm at pH of about 4.

In some embodiments, in any of the pharmaceutical compositions described above, the amount of the at least one ionizable cationic lipid relative to the total components of the TCV may be about 10 mol % to about 70 mol %, about 10 mol % to about 60 mol %, about 10 mol % to about 50 mol %, about 10 mol % to about 40 mol %, about 10 mol % to about 30 mol %, about 15 mol % to about 25 mol %, about 18 mol % to about 22 mol %, about 19 mol % to about 21 mol %, about 19.5 mol % to about 20.5 mol %, about 19.8 mol % to about 20.2 mol %, or about 20 mol %. In particular embodiments, the amount of the at least one ionizable cationic lipid relative to the total components of the TCV may be about 20 mol %.

In some embodiments, in any of the pharmaceutical compositions described above, the amount of the at least one ionizable cationic lipid relative to the total components of the TCV may be about 10 mol % to about 70 mol %, about 20 mol % to about 70 mol %, about 30 mol % to about 70 mol %, about 40 mol % to about 70 mol %, about 40 mol % to about 60 mol %, about 45 mol % to about 55 mol %, about 48 mol % to about 52 mol %, about 49 mol % to about 51 mol %, about 49.5 mol % to about 50.5 mol %, about 49.8 mol % to about 50.2 mol %, or about 50 mol %. In particular embodiments, the amount of the at least one ionizable cationic lipid relative to the total components of the TCV may be about 50 mol %.

In some embodiments, in any of the pharmaceutical compositions described above, the amount of the at least one helper lipid relative to the total components of the TCV may be about 10 mol % to about 60 mol %, about 10 mol % to about 50 mol %, about 10 mol % to about 40 mol %, about 20 mol % to about 40 mol %, about 25 mol % to about 35 mol %, about 28 mol % to about 32 mol %, about 29 mol % to about 31 mol %, about 29.5 mol % to about 30.5 mol %, about 29.8 mol % to about 30.2 mol %, or about 30 mol %. In particular embodiments, the amount of the at least one helper lipid relative to the total components of the TCV may be about 30 mol %.

In some embodiments, in any of the pharmaceutical compositions described above, the amount of the at least one phospholipid relative to the total components of the TCV may be about 5 mol % to about 65 mol %, about 5 mol % to about 55 mol %, about 5 mol % to about 45 mol %, about 5 mol % to about 35 mol %, about 5 mol % to about 25 mol %, about 5 mol % to about 15 mol %, about 8 mol % to about 12 mol %, about 9 mol % to about 11 mol %, about 9.5 mol % to about 10.5 mol %, about 9.8 mol % to about 10.2 mol %, or about 10 mol %. In particular embodiments, the amount of the at least one phospholipid relative to the total components of the TCV may be about 10 mol %.

In some embodiments, in any of the pharmaceutical compositions described above, the amount of the at least one cholesterol or cholesterol derivative relative to the total components of the TCV may be about 20 mol % to about 60 mol %, about 25 mol % to about 55 mol %, about 30 mol % to about 50 mol %, about 35 mol % to about 45 mol %, about 38 mol % to about 42 mol %, about 39 mol % to about 41 mol %, about 39.5 mol % to about 40.5 mol %, about 39.8 mol % to about 40.2 mol %, or about 40 mol %, or about 39%. In particular embodiments, the amount of the at least one cholesterol or cholesterol derivative relative to the total components of the TCV may be about 40 mol % or about 39%.

In some embodiments, in any of the pharmaceutical compositions described above, the amount of the at least one PEG or PEG-lipid relative to the total components of the TCV may be about 0.1 mol % to about 5 mol %, 0.1 mol % to about 4 mol %, 0.1 mol % to about 3 mol %, 0.1 mol % to about 2 mol %, 0.5 mol % to about 1.5 mol %, 0.8 mol % to about 1.2 mol %, 0.9 mol % to about 1.1 mol %, or about 1 mol %. In particular embodiments, the amount of the at least one PEG-lipid relative to the total components of the TCV may be about 1 mol %.

In some embodiments, in any of the pharmaceutical compositions described above, the TCV may comprise, essentially consist of, or consist of: (i) at least one ionizable cationic lipid, which is optionally DODMA; (ii) at least one helper lipid, which is optionally DOPE; (iii) at least one phospholipid, which is optionally DSPC; and (iv) at least one cholesterol or cholesterol derivative. In particular embodiments, the amounts of the at least one ionizable cationic lipid, the at least one helper lipid, the at least one phospholipid, and the at least one cholesterol or cholesterol derivative, relative to the total components of the TCV, may be about 20 mol %, about 30 mol %, about 10 mol %, and about 40 mol %, respectively. In a particular embodiment, the TCV may comprise, essentially consist of, or consist of, DODMA, DOPE, DSPC, cholesterol, with amounts (relative to the total components of the TCV) of about 20 mol %, about 30 mol %, about 10 mol %, and about 40 mol %, respectively.

In some embodiments, in any of the pharmaceutical compositions described above, the TCV may comprise, essentially consist of, or consist of: (i) at least one ionizable cationic lipid, which is optionally DODMA; (ii) at least one helper lipid, which is optionally DOPE; (iii) at least one phospholipid, which is optionally DSPC; (iv) at least one cholesterol or cholesterol derivative; and (v) at least one PEG or PEG-lipid, which is optionally PEG-DMG. In particular embodiments, the amounts of the at least one ionizable cationic lipid, the at least one helper lipid, the at least one phospholipid, the at least one cholesterol or cholesterol derivative, and the at least one PEG or PEG-lipid, relative to the total components of the TCV, may be about 20 mol %, about 30 mol %, about 10 mol %, about 39 mol %, and about 1 mol %, respectively.

In some embodiments, the TCV may be substantially, essentially, or entirely free of ethanol, methanol, isopropanol, tetrahydrofuran (THF), dimethyl sulfoxide (DMSO), dimethyl formamide (DMF), and acetonitrile (ACN). In particular embodiments, optionally wherein the TCV is substantially, essentially, or entirely free of organic solvents and detergents. In particular embodiments, the TCV may be substantially, essentially, or entirely free of destabilizing agents. In particular embodiments, the TCV may be stable for prolonged periods of time at about 1 to about 40° C., about 5 to about 35° C., about 10 to about 30° C., or about 15 to about 25° C.

In some embodiments, the TCV or the pharmaceutical composition may further comprise and/or be stored in the presence of at least one cryoprotectant. In certain embodiments, the cryoprotectant may comprise a sugar-based molecule, which is optionally sucrose, trehalose, or a combination thereof. In particular embodiments, the concentration of the cryoprotectant may be about 1% to about 40%, about 3% to about 30%, about 5% to about 30%, about 10% to about 20%, or about 15%. In a particular embodiment, the concentration of the cryoprotectant may be about 10% to about 20%. In certain embodiments, the TCV may be stable at a freezing temperature, optionally at about −20° C. or about −80° C. In certain embodiments, the TCV may be stable at a freezing temperature for at least about one week, at least about two weeks, at least about three weeks, at least about a month, at least about two months, at least about four months, at least about five months, at least about six months, at least about nine months, at least about a year, or at least about two years, or longer. In certain embodiments, the TCV may be stable at a freezing temperature for about one week to about two year, about two weeks to about a year, about three weeks to about nine months, about one to about six months, about one to five months, about one to four months, about one to three months, or about two months. In a particular embodiment, the TCV or the pharmaceutical composition may further comprise and/or be stored in the presence of about 10% to about 20% sucrose and may be stable at about −80° C. for at least about two months.

In some embodiments, in any of the pharmaceutical composition described above, the at least one SCD-associated gene may comprise one or more genes selected from the group consisting of HBB (the sickle cell hemoglobin (HbS) variant, also known as the βS allele), BCL11A, KLF1, SOX6, GATA1, NF-E4 (or NFE4), COUP-TF, NR2C1 (also known as TR2), NR2C2 (also known as TR4), genes encoding members of the MBD2 protein complex, IKZF1 (also known as Ikaros), genes encoding other members of PYR complex (CHD4, HDAC2, RBBP7, SMARCB1, SMARCC1, SMARCC2, SMARCD1, and SMARCE1), BRG1, and genes that directly or indirectly modulate the expression thereof.

In particular embodiments, the at least one SCD-associated gene may be HBB (such as the sickle cell hemoglobin (HbS) variant of HBB, also known as the βS allele or the hemoglobin C (HbC) variant of HBB), which optionally comprises the polynucleotide sequence of SEQ ID NO: 11, 21, or 31 and/or encoding the amino acid sequence of SEQ ID NO: 1, 2, or 3, and/or a promoter or enhancer region of HBB.

In particular embodiments, the at least one SCD-associated gene may be BCL11A, optionally encoding the amino acid sequence of SEQ ID NO: 6, and/or a promoter or enhancer region of BCL11A, preferably the erythroid-enhancer region (EER) of BCL11A.

In particular embodiments, the at least one SCD-associated gene may be KLF1, optionally encoding the amino acid sequence of SEQ ID NO: 7, and/or a promoter or enhancer region of KLF1.

In particular embodiments, the at least one SCD-associated gene may be HBG1, optionally encoding the amino acid sequence of SEQ ID NO: 8, and/or a promoter or enhancer region of HBG1. In particular embodiments, the at least one SCD-associated gene may be HBG2, optionally encoding the amino acid sequence of SEQ ID NO: 9, and/or a promoter or enhancer region of HBG2.

In some embodiments, in any of the pharmaceutical composition described above, the gene editing may be mediated by a protease, nuclease, endonuclease, meganuclease, zinc finger nuclease (ZFN), transcription activator-like nuclease (TALEN), or clustered regularly interspaced short palindromic repeats (CRISPR)-associated (Cas) nuclease, optionally resulting in at least one nucleic acid insertion, deletion, or replacement (e.g., resulting in a nonsense, missense, or silent mutation) in the at least one SCD-associated gene.

In some embodiments, in any of the pharmaceutical composition described above, the at least one cargo capable of effecting gene editing may comprise, essentially consist of, or consist of: (a) a Cas nuclease, a RNA encoding a Cas nuclease, or a nucleic acid such as a DNA or RNA encoding a Cas nuclease; and (b) a guide RNA (gRNA) comprising a target-complementary sequence which is complementary to a target sequence within the at least one SCD-associated gene and/or a promoter or enhancer thereof, or a nucleic acid encoding said gRNA.

In some embodiments, the Cas nuclease may be selected from the group consisting of Cas 9, Cas3, Cas8a2, Cas8b, Cas8c, Cas10, Csx11, Cas12, Cas12a or Cpf1, Cas13, Cas13a, C2c1, C2c3, and C2c2. In some embodiments, the Cas nuclease may be a class 2 Cas nuclease, optionally a type V or type VI Cas nuclease. In particular embodiments, the Cas nuclease may be Cas 9. In particular embodiments, Cas9 may be Cas9 of Streptococcus pyogenes (SpCas9), Staphylococcus aureus Cas9 (SaCas9), Streptococcus thermophilus (StCas9), Neisseria meningitidis (NmCas9), Francisella novicida (FnCas9), Campylobacter jejuni (CjCas9), Streptococcus canis (ScCas9), Staphylococcus auricularis (SauriCas9), or any engineered variants thereof, including SaCas9-HF, SpCas9-HF1, KKHSaCas9, eSpCas9, HypaCas9, FokI-Fused dCas9, xCas9, SpRY (variant of SpCas9), and SpG (variant of SpCas9). In particular embodiments, the Cas9 may comprise any one of SEQ ID NOS: 150-161.

In some embodiments, the gRNA may be a single guide RNA (sgRNA) comprising (1) a crRNA sequence comprising the target-complementary sequence and a crRNA backbone sequence and (2) a trans-activating CRISPR RNA (tracrRNA) sequence in a single strand. In certain embodiments, the crRNA sequence and the tracrRNA sequence may be linked via a linker optionally comprising SEQ ID NO: 139. In certain embodiments, the gRNA may comprise the target-complementary sequence followed by a sgRNA backbone sequence of any of SEQ ID NOS: 141-144, optionally wherein the sgRNA backbone sequence may be followed by one or more uracils, further optionally 1-10 uracils.

In some embodiments, the gRNA may be a dual guide RNA (dgRNA) formed by hybridization between (1) a crRNA sequence comprising the target-complementary sequence and a crRNA backbone sequence and (2) a tracrRNA. In certain embodiments, the crRNA backbone sequence and the tracrRNA may comprise SEQ ID NOS: 145 and 146, respectively, or SEQ ID NOS: 147 and 148, respectively.

In some embodiments, in any of the pharmaceutical composition described above, the at least one cargo may comprise, essentially consist of, or consist of a ribonucleoprotein (RNP), which is a complex of the gRNA and the Cas nuclease. In certain embodiments, the RNP may be any of the RNPs described above or herein.

In some embodiments, the RNP may be formed by mixing Cas9 and gRNA at an approximately equimolar ratio. In some embodiments, the mixing may be for about 5 minutes,

In some embodiments, in any of the pharmaceutical composition described above, the pharmaceutical composition or the at least one cargo may further comprise a DNA repair template, which optionally may be single stranded or double stranded.

In some embodiments, the at least one cargo (which may comprise the RNP or the RNP and the DNA repair template) encapsulated in the TCV may be obtained by: (i) providing an aqueous solution comprising the TCV, optionally wherein the pH of the aqueous solution is about 3 to about 8, further optionally about 4 to about 7.5; and (ii) mixing the at least one cargo with the aqueous solution, wherein mixing is effected under conditions suitable for the at least one cargo to be encapsulate within the TCV. In some embodiments, the mixing may comprise gentle mixing (optionally repeated manual reciprocation of the TCV-generating fluid in a pipette), micromixing optionally using a staggered herringbone micromixer (SHM) or T-junction or Y-junction mixing, or extrusion. In some embodiments, the mixing time may be about 0.1 second to about 20 minutes.

In some embodiments, the aqueous solution of step (i) may be substantially, essentially, or entirely free of ethanol, methanol, isopropanol, tetrahydrofuran (THF), dimethyl sulfoxide (DMSO), dimethyl formamide (DMF), and acetonitrile (ACN). In some embodiments, the aqueous solution of step (i) may be substantially, essentially, or entirely free of organic solvents and detergents, further optionally substantially, essentially, or entirely free of destabilizing agents.

In some embodiments, the mixing of step (ii) may be performed substantially, essentially, or entirely free of ethanol, methanol, isopropanol, tetrahydrofuran (THF), dimethyl sulfoxide (DMSO), dimethyl formamide (DMF), and acetonitrile (ACN). In some embodiments, the mixing of step (ii) may be performed substantially, essentially, or entirely free of organic solvents and detergents. In particular embodiments, the mixing of step (ii) may be performed substantially, essentially, or entirely free of destabilizing agents. In a particular embodiment, the final ethanol concentration after encapsulation may be 5% (v/v) or below. In a yet particular embodiment, the final ethanol concentration after encapsulation may be 0.5% (v/v) or below.

In some embodiments, the size of the TCV after encapsulation of the at least one cargo may be in a range of about 80 nm to about 1000 nm and/or in arrange of about 100 nm to about 250 nm, at pH of about 7.5.

In some embodiments, the at least one cargo (which comprises the RNP or the RNP and the DNA repair template) encapsulated in the TCV may be comprised in a matrix vesicle, which is optionally for gradual release of the TCV.

In some embodiments, in the pharmaceutical composition according to the present disclosure, the at least one SCD-associated gene may comprise or consist of HBB (such as the sickle cell hemoglobin (HbS) variant of HBB, also known as the βS allele, or the hemoglobin C (HbC) variant of HBB) and/or a promoter or enhancer region of HBB. In particular embodiments, the gRNA may direct the Cas protein to and hybridize to a target sequence, which may be located between nucleotide positions 5225464 to 5227071 of Chromosome 11 (according to Gene Assembly GRCh38.p13, positive or negative strand) and which may optionally be within the polynucleotide sequence of SEQ ID NO: 11, 21, or 31 or the sequence complementary thereto. In particular embodiments, the gRNA may direct the Cas protein to and hybridize to a target sequence, which may be located within or overlapping with exon 1 of HBB.

In some embodiments, the pharmaceutical composition or the at least one cargo may further comprise a DNA repair template which may allow for a knock-in of or correction to the wildtype HBB gene sequence (SEQ ID NO: 11) or the polynucleotide sequence encoding the wildtype beta-globin amino acid sequence (SEQ ID NO: 1).

In some embodiments, the at least one SCD-associated gene may comprise or consist of BCL11A and/or the erythroid-enhancer region (EER) of BCL11A and/or a promoter or enhancer region of BCL11A. In certain embodiments, the gRNA may direct the Cas protein to and hybridize to a target sequence, which may be located between nucleotide positions 60450520 to 60/553,654 of Chromosome 2 (according to Gene Assembly GRCh38.p13, positive or negative strand) and/or a promoter or enhancer region of BCL11A.

In some embodiments, the at least one SCD-associated gene may comprise or consist of KLF1 and/or a promoter or enhancer region of KLF1. In some embodiments, the gRNA may direct the Cas protein to and hybridize to a target sequence, which may be located between nucleotide positions 12884422 to 12887201 of Chromosome 19 (according to Gene Assembly GRCh38.p13, positive or negative strand) and/or a promoter or enhancer region of KLF1.

In some embodiments, the at least one SCD-associated gene may comprise or consist of HBG1 and/or a promoter or enhancer region of HBG1, preferably in the BCL11A-binding site thereof. In some embodiments, the gRNA may direct the Cas protein to and hybridize to a target sequence, which may be located between nucleotide positions 5248269 to 5249857 of Chromosome 11 (according to Gene Assembly GRCh38.p14, positive or negative strand), preferably in the BCL11A-binding site thereof.

In some embodiments, the at least one SCD-associated gene may comprise or consist of HBG2 and/or a promoter or enhancer region of HBG2, preferably in the BCL11A-binding site thereof. In some embodiments, the gRNA may direct the Cas protein to and hybridize to a target sequence, which may be located between nucleotide positions 5253188 to 5254781 of Chromosome 11 (according to Gene Assembly GRCh38.p14, positive or negative strand), preferably in the BCL11A-binding site thereof.

In some embodiments, in any of the pharmaceutical composition for effecting gene editing to HBB, the target sequence may be or comprise SEQ ID NO: 24 or the first 17, 18, or 19 nucleotides from the 5′ end of SEQ ID NO: 24, and/or the target-complementary sequence may comprise the polynucleotide sequence of SEQ ID NO: 25, or the first 17, 18, or 19 nucleotides thereof from the 3′ end of SEQ ID NO: 25. In some embodiments, in any of the pharmaceutical composition for effecting gene editing to HBB, the target sequence may be or comprise SEQ ID NO: 44 or the first 17, 18, or 19 nucleotides from the 5′ end of SEQ ID NO: 44 such as SEQ ID NO: 46, and/or the target-complementary sequence may comprise the polynucleotide sequence of SEQ ID NO: 45, or the first 17, 18, or 19 nucleotides thereof from the 3′ end of SEQ ID NO: 45 such as SEQ ID NO: 47. In some embodiments, in any of the pharmaceutical composition for effecting gene editing to HBB, the target sequence may be or comprise SEQ ID NO: 48 or the first 17, 18, or 19 nucleotides from the 5′ end of SEQ ID NO: 48, and/or the target-complementary sequence may comprise the polynucleotide sequence of SEQ ID NO: 49, or the first 17, 18, or 19 nucleotides thereof from the 3′ end of SEQ ID NO: 49.

Optionally, such a pharmaceutical composition or the at least one cargo may further comprise a DNA repair template, which optionally comprise: (I) a single-strand oligo DNA nucleotide molecule (ssODN) comprising or consisting of a 5′ homology arm, a central region, and a 3′ homology arm, or (II) a double-strand DNA molecule, which comprises a first strand comprising any of the ssODN sequences of (I) and a second strand complementary to the first strand.

As for ssODNs, in some embodiments, the 5′ homology arm may comprise or consist of (i-1) the sequence of SEQ ID NO: 112, (i-2) the sequence corresponding to the first nucleotide to at least the 20th nucleotide (e.g., at least the 30th, such as to the 39th, at least the 40th, such as to the 49th, or at least the 50th, such as to the 50th or the 59th) counting from the 3′-end of SEQ ID NO: 112, (i-3) or a sequence comprising at least one (such as one, two, three, four, five, six, seven, eight, nine, or ten) silent mutation(s) relative to the sequence of (i-1) or (i-2). In some embodiments, the central region may have the sequence of 5′-CTCA-3′, 5′-TTCA-3′, 5′-CTCT-3′, 5′-TTCT-3′, 5′-CTCC-3′, 5′-TTCC-3′, 5′-CTCG-3′, or 5′-TTCG-3′. In some embodiments, the 3′ homology arm may comprise or consist of (i-1) the sequence of SEQ ID NO: 122, (i-2) the sequence corresponding to the first nucleotide to at least the 20th nucleotide (e.g., at least the 30th, such as to the 37th, at least the 40th, such as to the 47th, or at least the 50th, such as to the 57th) counting from the 5′-end of SEQ ID NO: 122, (i-3) or a sequence comprising at least one (such as one, two, three, four, five, six, seven, eight, nine, or ten) silent mutation(s) relative to the sequence of (iii-1) or (iii-2). In particular embodiments, the ssODN may comprise the consist of the sequence of any of SEQ ID NOs: 170, 172, 174, 176, and 101-108. In a particular embodiment, the ssODN may comprise or consist of the sequence of SEQ ID NO: 101 or 102. Alternatively, the sequence of the ssODN may be fully complementary to the sequence any of the ssODNs described above. In particular embodiments, the sequence of the ssODN may be or may comprise any of SEQ ID NOs: 169, 171, 173, and 175. In particular embodiments, a silent mutation if included may be at the 12th nucleotide of SEQ ID NO: 112 (for example a G-to-C mutation) or the corresponding nucleotide position of a sequence complementary to SEQ ID NO: 112 (for example a C-to-G mutation).

As for the double-strand DNA molecules, a double-strand DNA molecule may comprise a first strand comprising any of the ssODN sequences described above and a second strand complementary to the first strand.

In some embodiments, in any of the pharmaceutical composition for effecting gene editing to BCL11A, the target sequence may be or comprise SEQ ID NO: 64 or the first 17, 18, or 19 nucleotides from the 5′ end of SEQ ID NO: 64, and/or the target-complementary may comprise the polynucleotide sequence of SEQ ID NO: 65, or the first 17, 18, or 19 nucleotides thereof from the 3′ end of SEQ ID NO: 65. In some embodiments, the target sequence may be or comprise SEQ ID NO: 66 or the first 17, 18, or 19 nucleotides from the 5′ end of SEQ ID NO: 66, and/or the target-complementary sequence may comprise the polynucleotide sequence of SEQ ID NO: 67 or the first 17, 18, or 19 nucleotides thereof from the 3′ end of SEQ ID NO: 67. In some embodiments, the target sequence may be or comprise SEQ ID NO: 68 or the first 17, 18, or 19 nucleotides from the 5′ end of SEQ ID NO: 68, and/or the target-complementary sequence may comprise the polynucleotide sequence of SEQ ID NO: 69 or the first 17, 18, or 19 nucleotides thereof from the 3′ end of SEQ ID NO: 69.

In some embodiments, in any of the pharmaceutical composition for effecting gene editing to KLF1, the target sequence may be or comprise SEQ ID NO: 74 or the first 17, 18, or 19 nucleotides from the 5′ end of SEQ ID NO: 74, and/or the target-complementary sequence may comprise the polynucleotide sequence of SEQ ID NO: 75 or the first 17, 18, or 19 nucleotides thereof from the 3′ end of SEQ ID NO: 75. In some embodiments, the target sequence may be or comprise SEQ ID NO: 76 or the first 17, 18, or 19 nucleotides from the 5′ end of SEQ ID NO: 76, and/or the target-complementary sequence may comprise the polynucleotide sequence of SEQ ID NO: 77 or the first 17, 18, or 19 nucleotides thereof from the 3′ end of SEQ ID NO: 77.

In some embodiments, in any of the pharmaceutical composition for effecting gene editing to HBG1 and/or HBG2, the target sequence may be or comprise SEQ ID NO: 84 or the first 17, 18, or 19 nucleotides from the 5′ end of SEQ ID NO: 84, and/or the target-complementary sequence may comprise the polynucleotide sequence of SEQ ID NO: 85 or the first 17, 18, or 19 nucleotides thereof from the 3′ end of SEQ ID NO: 85.

In some embodiments, the pharmaceutical composition may comprise a RNP comprising any of the gRNAs described above or herein.

In some embodiments, the pharmaceutical composition may comprise at least one cargo capable of altering the expression of a target gene. In some embodiments, the cargo may comprise, essentially consist of, or consist of a nucleic acid molecule. In some embodiments, the nucleic acid molecule may be a ribonucleic acid (RNA), a single or double stranded RNA, a small interfering RNA (siRNA), a short hairpin RNA, a microRNA (miRNA), a messenger RNA (mRNA), a deoxyribonucleic acid (DNA), a double or single stranded DNA, a plasmid DNA, a complementary DNA (cDNA), and/or a locked nucleic acid.

Again, in some embodiments, the at least one cargo encapsulated in the TCV may be obtained by: (i) providing an aqueous solution comprising the TCV, optionally wherein the pH of the aqueous solution is about 3 to about 8, further optionally about 4 to about 7.5; and (ii) mixing the at least one cargo with the aqueous solution. In some embodiments, the mixing may be effected under conditions suitable for the at least one cargo to be encapsulate within the TCV. In some embodiments, the mixing may comprise gentle mixing (optionally repeated manual reciprocation of the TCV-generating fluid in a pipette), micromixing optionally using a staggered herringbone micromixer (SHM) or T-junction or Y-junction mixing, or extrusion. In some embodiments, the mixing time may be about 0.1 second to about 20 minutes.

In some embodiments, the aqueous solution of step (i) may be substantially, essentially, or entirely free of ethanol, methanol, isopropanol, tetrahydrofuran (THF), dimethyl sulfoxide (DMSO), dimethyl formamide (DMF), and acetonitrile (ACN), optionally substantially, essentially, or entirely free of organic solvents and detergents. In particular embodiments, the aqueous solution of step (i) may be substantially, essentially, or entirely free of destabilizing agents.

In some embodiments, the mixing of step (ii) may be performed substantially, essentially, or entirely free of ethanol, methanol, isopropanol, tetrahydrofuran (THF), dimethyl sulfoxide (DMSO), dimethyl formamide (DMF), and acetonitrile (ACN). In some embodiments, the mixing of step (ii) may be performed substantially, essentially, or entirely free of organic solvents and detergents. In particular embodiments, the mixing of step (ii) may be performed substantially, essentially, or entirely free of destabilizing agents.

In particular embodiments, the final ethanol concentration after encapsulation may be 5% (v/v) or below, preferably 0.5% (v/v) or below.

In some embodiments, in any of the above-described pharmaceutical composition, the pharmaceutical composition may comprise at least another cargo. In some instances, the at least another cargo may be encapsulated in the TCV encapsulating at least one cargo capable of effecting gene editing or gene expression alteration as describe above. Or, in some instances, the at least another cargo may be encapsulated in a different or separate TCV. Or, in other instances, the at least another cargo may not be encapsulated in a TCV. Regardless of whether the encapsulation status of the at least another cargo, the at least another cargo may be according to any of the at least one cargo capable of effecting gene editing and/or gene expression alteration.

In some embodiments, in any of the above-described pharmaceutical composition, the pharmaceutical composition may further comprise at least one agent that promotes stem cell mobilization. In some instances, the at least one agent that promotes stem cell mobilization may be selected from the group consisting of granulocyte colony-stimulating factor (G-CSF), granulocyte-macrophage colony-stimulating factor (GM-CSF), Plerixafor, stem cell factor (SCF), CXCR4 antagonists (e.g., POL6326, BKT-140, TG-0054), CXCL12 neutralizers (e.g., NOX-A12), Sphingosine-1-phosphate (SIP) antagonists (e.g., SEW2871), vascular cell adhesion molecule-1/Very Late Antigen 4 (VCAM/VLA-4) inhibitors (e.g., BIO 5192), parathyroid hormone, protease inhibitors (e.g., Bortezomib), Grop (e.g., SB-251353), and hypoxia inducible factor (HIF) stabilizers (e.g., FG-4497).

In some embodiments, in any of the above-described pharmaceutical composition, the pharmaceutical composition may further comprise at least one agent that promotes erythropoiesis. Om some instances, such an agent may be selected from the group consisting of SCF, GM-CSF, interleukin-3 (IL-3), interleukin-9 (IL-9), erythropoietin (EPO) (or an engineered EPO or EPO mimetic), TGF-beta, growth differentiating factor 11 (GDF11), Activin A, Transferrin (Tf), ferritin, ferroportin, hepcidin, vitamin B12, folic acid, and copper. In some other instances, the at least one agent that promotes erythropoiesis may be selected from the group consisting of GATA-1, STAT5A, STAT5B, MCL-1, BCL-xL, and HSP70, a RNA or DNA encoding thereof, and in some cases the agent may be encapsulated in the TCV encapsulating at least one cargo capable of effecting gene editing or gene expression alteration as describe above, or, alternatively, in a different (separate) TCV. In yet other instances, the at least one agent that promotes erythropoiesis may be an inhibitor or silencer of a negative regulator of erythropoiesis, and in some cases such a negative regulator may be selected from the group consisting of inhibin, TGF-beta, BID (a member of the BCL-2 family), Fas ligand, Fas, and caspases. In some cases, the negative regulator may be encapsulated in the TCV encapsulating at least one cargo capable of effecting gene editing or gene expression alteration as describe above or, alternatively, in a different (separate) TCV.

In some embodiments, in any of the above-described pharmaceutical composition, the TCV may comprise at least one targeting moiety which may allow the TCV to carry the at least one cargo preferentially into one or more target cells. In some embodiments, the one or more target cells may comprise hematopoietic stem cells (HSCs), hematopoietic stem and progenitor cells (HSPCs), multipotent progenitor cells (MPPs), common myeloid progenitors (CMPs), megakaryocyte-erythroid progenitors (MEPs), hematopoietic progenitor cells (HPCs), erythroid progenitors (e.g., burst-forming unit erythroid cells (BFU-Es), colony-forming unit erythroid cells (CFU-Es)), proerythroblasts, erythroblasts (basophilic erythroblasts, early erythroblasts (e.g., type I, type II), polychromatic erythroblasts, intermediate erythroblasts, acidophilic erythroblasts, late erythroblasts, normoblasts, reticulocytes (before nucleus expulsion), or any combinations thereof, In some preferred embodiments, the one or more target cells may comprise, may be, may essentially consist of, or consist of HSCs and/or HSPCs. In particular embodiments, the targeting moiety may be specific to, target CD34, and/or target CD34+ cells.

In another aspect, the present disclosure further provides a method for effecting gene editing and/or gene expression alteration in one or more target cells in vivo in a subject in need thereof.

In some embodiments, the pharmaceutical composition may be injected into the bone marrow of the subject.

In certain embodiments, the one or more target cells may comprise HSCs, HSPCs, MPPs, CMPs, MEPs, HPCs, erythroid progenitors (e.g., BFU-Es, CFU-Es), proerythroblasts, erythroblasts (basophilic erythroblasts, early erythroblasts (e.g., type I, type II), polychromatic erythroblasts, intermediate erythroblasts, acidophilic erythroblasts, late erythroblasts, normoblasts, reticulocytes (before nucleus expulsion), or any combinations thereof, preferably HSCs and/or HSPCs.

In certain embodiments, the subject has or has a risk of developing SCD, which may optionally be SCA, HbSC, or HbS β-thalassaemia.

In certain embodiments, the pharmaceutical composition may comprise, per mL, about 300 pmol to about 30000 pmol of the RNP or the nucleic acid molecule. In particular embodiments, the pharmaceutical composition may comprise, per mL, about 500 to about 10000 pmol, about 1000 to about 5000 pmol, about 2000 to about 4000 pmol, about 2500 to about 3000 pmol, or about 2700 pmol of the RNP or the nucleic acid molecule.

In certain embodiments, the injecting may comprise injecting the pharmaceutical composition in a continuous flow of about 25 mL to 125 mL per minute, In particular embodiments, the injecting may comprise about 25 mL to 50 mL per minute, about 50 mL to 100 mL per minute, about 100 mL to 125 mL per minute, about 40 mL to about 80 mL per minute, or about 50 mL to about 70 mL per minute.

In certain embodiments, the injecting may be a slow bolus push using an instrument with an intraosseous device or intramarrow needle optionally having a needle length of about 50 to about 100 mm or about 70 to about 80 nm.

In certain embodiments, the injecting may be effected, optionally two or more times, to reach a minimum of about 10%, about 15%, about 20%, about 30%, or an about final 15-30% or about final 20-40% HSCs and HSPCs with successful gene editing and/or gene expression alteration among the total HSCs and HSPCs in the bone marrow.

In certain embodiments, the injecting may be effected two or more times, optionally about 3-5 time, optionally about once a week, about every 2 weeks, or about every 3 weeks, about once a month, about every 3 months, about every 6 months, or about once per year.

In certain embodiments, the bone marrow the composition may be injected into may be the bone of tibia, femur, sternum, skull, ribs, pelvis (e.g., iliac), or any combinations thereof.

In certain embodiments, the subject may be of any age and/or at any stage of the disease. For example, the subject may be in the immediate post-natal period, optionally about 6 weeks old or younger, may be about 3 month old or younger, may still comprises sufficient amount of fetal hemoglobin (HbF) relative to adult hemoglobin (HbA) (e.g., HbF:HbA is about 2:1, about 1:1, about 1:2, about 1:3, about 1:4, about 1:5, or about 1:10), and/or may not have fully developed SCD and may be prior to manifesting a symptom or complication.

In some embodiments, in the method of effecting gene editing and/or gene expression alteration in one or more target cells in vivo in a subject in need thereof, the method may comprise: (I) administering, optionally intravenously, to the subject at least one agent that promotes stem cell mobilization (from the bone marrow to the peripheral circulation); and (II) injecting, optionally intravenously, any of the pharmaceutical compositions described above into the peripheral circulation of the subject.

In some instances, the at least one agent that promotes stem cell mobilization may, for example, selected from the group consisting of G-CSF (filgrastim), GM-CSF, Plerixafor, SCF, CXCR4 antagonists (e.g., POL6326, BKT-140, TG-0054), CXCL12 neutralizers (e.g., NOX-A12), Sphingosine-1-phosphate (SIP) antagonists (e.g., SEW2871), VCAM/VLA-4 inhibitors (e.g., BIO 5192), parathyroid hormone, protease inhibitors (e.g., Bortezomib), Grop (e.g., SB-251353), hypoxia inducible factor (HIF) stabilizers (e.g., FG-4497), and any combinations thereof.

In certain embodiments, the one or more target cells may comprise HSCs, HSPCs, MPPs, CMPs, MEPs, HPCs, erythroid progenitors (e.g., BFU-Es, CFU-Es), proerythroblasts, erythroblasts (basophilic erythroblasts, early erythroblasts (e.g., type I, type II), polychromatic erythroblasts, intermediate erythroblasts, acidophilic erythroblasts, late erythroblasts, normoblasts, reticulocytes (before nucleus expulsion), or any combinations thereof. In a particular embodiment the one or more target cells may comprise, may be, may essentially consist of, or consist of HSCs and/or HSPCs.

In some instances, the subject may have or may have a risk of developing SCD, which optionally may be SCA, HbSC, or HbS β-thalassaemia.

In certain embodiments, the administering at least one agent that promotes stem cell mobilization may comprise intravenous (IV) administration of G-CSF followed by intravenous administration of plerixafor prior to said injecting. In particular embodiments, the dosing of G-CSF may be about 5-30 μg/kg/day, preferably about 10 μg/kg/day, for about 3-5 days, preferably 4 days. In particular embodiments, the dosing of plerixafor may start once the peripheral blood CD34+ cells are <20 cells/μL and/or on the day of the last G-CSF administration or the following day. In particular embodiments, the dosing of plerixafor may be about 0.1-0.5 mg/kg, preferably about 0.2-0.3 mg/kg or about 0.24 mg/kg.

In certain embodiments, the pharmaceutical composition which is to be administered to the peripheral circulation of the subject may comprise, per mL, about 300 pmol to about 30000 pmol of the RNP or the nucleic acid molecule. In particular embodiments, the pharmaceutical composition which is to be administered to the peripheral circulation of the subject may comprise, per mL, about 500 to about 10000 pmol, about 1000 to about 5000 pmol, about 2000 to about 4000 pmol, about 2500 to about 3000 pmol, or about 2700 pmol of the RNP or the nucleic acid molecule.

In certain embodiments, the injecting any of the pharmaceutical compositions may starts once the peripheral blood CD34+ cells are 60 cells/μL or more. In certain embodiments, the injecting may be a single injection, optionally about 3-7 days, about every 3-7 days, about 4-6 days, about every 4-6 days, about 5 days, or about every 5 days after the last plerixafor administration. In certain embodiments, the injecting may occur once daily for one week following the last plerixafor administration.

In certain embodiments, the injecting may comprise injecting the pharmaceutical composition in a continuous flow of about 25 mL to 125 mL per minute. In particular embodiments, the injecting may comprise about 25 mL to 50 mL per minute, about 50 mL to 100 mL per minute, about 100 mL to 125 mL per minute, about 40 mL to about 80 mL per minute, or about 50 mL to about 70 mL per minute.

In certain embodiments, the combination of the administration of at least one agent that promotes stem cell mobilization and the injection of any of the pharmaceutical compositions described herein may be effected, optionally two or more times, to reach a minimum of about 10%, about 15%, about 20%, about 30%, or an about final 15-30% or about final 20-40% HSCs and HSPCs with successful gene editing and/or gene expression alteration among the total HSCs and HSPCs in the peripheral circulation.

In certain embodiments, the combination of the administration of at least one agent that promotes stem cell mobilization and the injection of any of the pharmaceutical compositions described herein may be effected, optionally two or more times, to reach a minimum of about 10%, about 15%, about 20%, about 30%, or an about final 20-30% increase in the peripheral HSCs and HSPCs expressing HbF, or a minimum of about 10%, about 15%, about 20%, about 30%, or an about final 20-30% increase in the total HbF expression levels in the total HSCs and HSPCs in the peripheral circulation, optionally wherein the SCD-associated gene is BCL11A, HBG1, HBG2, or KLF1.

In certain embodiments, the combination of the administration of at least one agent that promotes stem cell mobilization and the injection of any of the pharmaceutical compositions described herein may be effected two or more times. In particular embodiments, the combination may be effected about 3-5 time, about once a week, about every 2 weeks, or about every 3 weeks, about once a month, about every 3 months, about every 6 months, or about once per year.

In certain embodiments, the subject may be at any age and/or at any disease stage. In particular embodiments, the subject may be in the immediate post-natal period, optionally about 6 weeks old or younger, may be about 3 month old or younger, may still comprises sufficient amount of HbF relative to HbA (e.g., HbF:HbA is about 2:1, about 1:1, about 1:2, about 1:3, about 1:4, about 1:5, or about 1:10), and/or may not have fully developed SCD and is prior to manifesting a symptom or complication.

In another aspect, the present disclosure further provides a method for preventing, ameliorating, or treating a disease, which may relate to cells of bone marrow origin and/or cells of the bone marrow. In some embodiments, the disease may be SCD, which optionally may be SCA, HbSC, or HbS β-thalassaemia, in a subject in need thereof.

In some embodiments, the method for preventing, ameliorating, or treating a disease may comprise any of the in vivo methods described herein in which the pharmaceutical composition is injected into the bone marrow of the subject and/or the in vivo method in which the pharmaceutical composition is injected into the peripheral circulation (e.g., IV) of the subject.

In certain embodiments, the effect of the method may be evaluated based on any appropriate parameters (and/or any combinations thereof) that indicate successful gene editing and/or gene expression alteration and/or the associated improvement in any of the disease symptoms.

In particular embodiments, the method may be evaluated based on % HSCs and HSPCs in the blood with successful gene editing and/or gene expression alteration. In particular embodiments, the method may be evaluated based on the number of HSCs and HSPCs in the blood with successful gene editing and/or gene expression alteration. In particular, the method may be evaluated based on % HSCs and HSPCs expressing HbF (e.g., when the SCD-associated gene is BCL11A or KLF1). In particular embodiments, the method may be evaluated based on the number of HSCs and HSPCs expressing HbF, optionally wherein the SCD-associated gene is BCL11A, HBG1, HBG2, or KLF1. In particular embodiments, the method may be evaluated based on the expression level of the at least one SCD-associated gene or gene product or molecule, such as but not limited to beta-globin, beta-globin (HbS variant), gamma-globin 1, gamma-globin 2, HbF, HbA, BCL11A, and/or KLF1. In particular embodiments, the method may be evaluated based on changes in the symptom optionally pain, swelling of hands and feet, infection frequency, growth, and/or symptoms associated with vision.

In certain embodiments, the method further comprises administering at least one agent that promotes erythropoiesis. In particular embodiments, the at least one agent that promotes erythropoiesis may be selected from the group consisting of SCF, GM-CSF, IL-3, IL-9, EPO (or an engineered EPO or EPO mimetic), TGF-beta, GDF11, Activin A, Tf, ferritin, ferroportin, hepcidin, vitamin B12, folic acid, copper, and any combinations thereof. In particular embodiments, the at least one agent that promotes erythropoiesis may be selected from the group consisting of GATA-1, STAT5A, STAT5B, MCL-1, BCL-xL, and HSP70, a RNA or DNA encoding thereof. Such an agent may be encapsulated in the TCV encapsulating the at least one cargo capable of effecting gene therapy and/or gene expression alteration. Alternatively, such an agent may be encapsulated in a TCV different or separate from the TCV encapsulating the at least one cargo capable of effecting gene therapy and/or gene expression alteration. In particular embodiments, the at least one agent that promotes erythropoiesis may be an inhibitor or silencer of a negative regulator of erythropoiesis. In a particular embodiment, optionally the negative regulator may be selected from the group consisting of inhibin, TGF-beta, BID (a member of the BCL-2 family), Fas ligand, Fas, and caspases, and any combinations thereof. Such an agent may be encapsulated in the TCV encapsulating the at least one cargo capable of effecting gene therapy and/or gene expression alteration. Alternatively, such an agent may be encapsulated in a TCV different or separate from the TCV encapsulating the at least one cargo capable of effecting gene therapy and/or gene expression alteration.

In certain embodiments, the method further comprises administering at least one other agent for treating SCD, which optionally comprises hydroxyurea, L-glutamine oral powder, crizanlizumab, a general pain medication, voxelotor, or any combination thereof.

In another aspect, the present disclosure further provides a method of manufacturing any of the pharmaceutical compositions described herein comprising a RNP.

In some embodiments, the method may comprise: (a) providing an aqueous solution comprising the TCV, optionally wherein the pH of the aqueous solution is about 3 to about 8, further optionally about 3.5 to about 7.5, about 3.5 to about 5.5, or about 4; and (b) mixing the at least one cargo with the aqueous solution under conditions suitable for the at least one cargo to be encapsulate within the TCV,

In certain embodiments, the RNP may be any of the RNPs described herein.

In certain embodiments, the mixing may be for about 0.1 second to about 20 minutes. In certain embodiments, the mixing may be via gentle mixing (optionally repeated manual reciprocation of the TCV-generating fluid in a pipette). In certain embodiments, the mixing may be via micromixing, optionally using a staggered herringbone micromixer (SHM) or T-junction or Y-junction mixing. In certain embodiments, the method may comprise extrusion.

### BRIEF DESCRIPTION OF THE DRAWINGS

FIG. 1 is a schematic of a transgenic reporter mouse design. Proof-of-concept gRNAs are designed, so that, when successful gene editing occurs, the reporter gene will be turned on.

FIGS. 2A-2C provide exemplary disruption of the erythroid-enhancer region (EER) of BCL11A.

FIG. 2A shows a schematic of an editing strategy used in Examples 12-13. Numbered boxes represent BCL11A exons of the indicated exon number, and lines between exons indicate introns. The dashed box represents the EER in its intact form. Each open rectangle above the EER represents a sgRNA portion comprising a target-complementary sequence (e.g., SEQ ID NO: 65, 67, or 69) which may hybridize to the EER. Each filled rectangle adjacent thereto indicates where the PAM sequence is in the target DNA. Upon hybridization of a sgRNA, spCas9 may mediate NHEJ, resulting in a disrupted EER (grid box). Transcription factor(s) may no longer bind to the disrupted EER in cells of the erythroid lineage, and transcription of BCL11A may be inhibited. BCL11A is a repressor of HBG1 and HBG2, and inhibition of BCL11A transcription may result in increased gamma-globin thereby restoring HbF production.

FIG. 2B provides exemplary results obtained in Example 12. HEK293 cells were treated with TCV-encapsulated RNPs comprising sgRNA targeting luciferase (using SEQ ID NO: 55), BCL11A EER1 (using SEQ ID NO: 65), or BCL11A EER2 (using SEQ ID NO: 69). The graph shows percent editing efficiency at the target sites. N=3 per treatment condition. One way ANOVA: p=0.0010. Dunnett's multiple comparison test: **p=0.0027, ***p=0.0007.

FIG. 2C provides exemplary results obtained in Example 13. HEK293 cells were treated with increasing amounts of TCV-encapsulated RNPs comprising sgRNA targeting luciferase (using SEQ ID NO: 55) or targeting BCL11A EER1 (using SEQ ID NO: 65). The graph shows percent editing efficiency at the target sites. N=3 per treatment condition. Two-way way ANOVA: treatment p<0.0001, dose p<0.0001, interaction p<0.0001. Tukey's multiple comparison test: ****p<0.0001.

FIGS. 3A-3D provide exemplary disruption of the BCL11A-binding site present in both the promoter of HBG1 and the promoter of HBG2.

FIG. 3A shows a schematic of an editing strategy used in Examples 14-15. Boxes with italic letters indicate the HBE, HBG2, HBG1, HBD, and HBB genes located in the β-like globin gene cluster. Dotted boxes are the promoters for HBG2 and HBG1. Each open rectangle above the promoter represents a sgRNA portion comprising a target-complementary sequence (e.g., SEQ ID NO: 85) which may hybridize to the promoter. Each filled rectangle adjacent thereto indicates where the PAM sequence is in the target DNA. Upon hybridization of a sgRNA, spCas9 may mediate NHEJ, resulting in a disrupted promoter (grid box). BCL11A (gamma-globin repressor) may no longer bind to the promotor of HBG1 and/or the promoter of HBG2, and transcription of HBG1 and/or HBG2 may be inhibited, thereby restoring HbF production.

FIG. 3B provides exemplary results obtained in Example 14. HEK293 cells were treated with TCV-encapsulated RNPs comprising sgRNA targeting (i) luciferase (using SEQ ID NO: 55) or (ii) the HBG promoters of HBG1 and HBG2 (using SEQ ID NO: 85). The graph shows percent editing efficiency at the target promoter site of HBG1 (left) and the target promoter site of HBG2 (right), when the sgRNA targeting (i) luciferase (circle) or (ii) the HBG promoters (square) was used. (N=3 per treatment condition. Two-way ANOVA: HBG promoter p<0.0001, treatment p<0.0001, interaction p=0.0021. Sidak's multiple comparison test: *p=0.0298, ****p<0.0001).

FIG. 3C provides exemplary editing efficiency results obtained in Example 15. HEK293 cells were treated with increasing amounts of TCV-encapsulated RNPs comprising sgRNA targeting luciferase (using SEQ ID NO: 55) or targeting the HBG promoters of HBG1 and HBG2 (using SEQ ID NO: 85). The left graph shows percent editing efficiency at the target promoter site of HBG1 when the sgRNA targeting (i) luciferase (left data set) or (ii) the HBG promoters (right data set) was used. N=3 per treatment condition. Two-way ANOVA: treatment p<0.0001, dose p<0.0001, interaction p<0.0001. Tukey's multiple comparison test: **p<0.01, ****p<0.0001. The right graph shows percent editing efficiency at the target promoter site of HBG2 when the sgRNA targeting (i) luciferase (left data set) or (ii) the HBG promoters (right data set) was used. N=3 per treatment condition. Two-way ANOVA: treatment p<0.0001, dose p<0.0001, interaction p<0.0001. Tukey's multiple comparison test: ****p<0.0001.

FIG. 3D provides exemplary editing event results obtained in Example 15. The graph provides a representative histogram showing distribution of specific editing events following treatment with the TCV-encapsulated RNP targeting the promoter region of HBG1 and HBG2 at 200 nM. The p values indicate whether the editing event occurred at a frequency higher than what would be expected by chance. P values were determined as described in Brinkman et al., Nucleic Acids Res. 2014 Dec. 16; 42(22):e168 (see, e.g., pages 2-3 such as “the sequence trace from the mutated DNA sample is assumed to be a linear combination of the wild-type and the modeled indel traces. This combination is then resolved by standard non-negative linear modeling, for which we used the R package nnls. R² is calculated to assess the goodness of fit. The p-value associated with the estimated abundance of each indel is calculated by a two tailed t-test of the variance-covariance matrix of the standard errors. In order to account for systematic differences between the sequence trace intensities of the control and mutated DNA, the fitting parameters are then multiplied by a constant factor such that their sum equals R².”).

FIGS. 4A-4C provide exemplary editing of and/or correction of mutant HBB exon 1.

FIG. 4A shows a schematic of strategy for editing and correcting mutant HBB exon 1 (e.g., containing a E-to-V mutation). Editing of HBB exon 1 exemplified in Examples 16-17 may be used as part of the strategy. Numbered boxes represent HBB exons of the indicated exon number. The checker box represents a mutation in HBB exon 1. Each open rectangle above HBB exon 1 represents a sgRNA portion comprising a target-complementary sequence (e.g., SEQ ID NO: 25, 45, 47, or 49) which may hybridize to HBB exon 1. Each filled rectangle adjacent thereto indicates where the PAM sequence is in the target DNA. A DNA template (e.g., ssODN) for correcting back to WT or non-disease causing exon 1 may be added. Upon hybridization of a sgRNA, spCas9 may cause cleavage at the target site and the DNA template may mediate HDR, resulting in a correction (slashed box) in HBB exon 1. Correction of mutant HBB exon 1 may result in production of WT or corrected beta-globin, thereby restoring normal HbA production.

FIG. 4B provides exemplary results obtained in Example 16. HEK293 cells were treated with TCV-encapsulated RNPs comprising sgRNA targeting luciferase (using SEQ ID NO: 55), HBB E6V 1A (using SEQ ID NO: 45), or HBB E6V 1B (using SEQ ID NO: 47). The graph shows percent editing efficiency at the target sites. N=3 per treatment condition. One way ANOVA: p<0.0001 Dunnett's multiple comparison test: ***p=0.0001, ****p<0.0001.

FIG. 4C provides exemplary results obtained in Example 17. HEK293 cells were treated with increasing amounts of TCV-encapsulated RNPs comprising sgRNA targeting luciferase (using SEQ ID NO: 55) or targeting HBB E6V 1A (using SEQ ID NO: 45). The graph shows percent editing efficiency at the target sites. N=3 per treatment condition. Two way ANOVA: treatment p<0.0001, dose p<0.0001, interaction p<0.0001. Tukey's multiple comparison test: ****p<0.0001.

### DETAILED DESCRIPTION OF THE INVENTION

The present disclosure provides, among other things, compositions and methods for preventing, ameliorating, and/or treating SCD. The present disclosure also provides compositions and methods for effecting gene editing and/or gene expression alteration in vivo in cells of bone marrow origin and/or cells in the bone marrow.

### Targets

### Target Diseases

In one aspect, a target disease according to the present disclosure may comprise a disease involving cells of bone marrow. In some embodiments, the disease may comprise SCD. In some embodiments, the disease may comprise sickle cell anemia (SCA), Sickle cell-hemoglobin C (HbSC), and HbS β-thalassaemia (also called β-thalassaemia).

### Target Cells

In one aspect, a target cell or target cells according to the present disclosure may comprise a cell or cells of bone marrow origin. In some embodiments, the target cell or target cells may comprise a cell or cells in the bone marrow origin. In some embodiments, the target cell or target cells may comprise a cell or cells capable of differentiating into a RBC. In some embodiments, the target cell or target cells may comprise HSCs, HSPCs, MPPs, CMPs, MEPs, HPCs, erythroid progenitors (e.g., BFU-E, CFU-E), proerythroblasts, erythroblasts (basophilic erythroblasts, early erythroblasts (e.g., type I, type II), polychromatic erythroblasts, intermediate erythroblasts, acidophilic erythroblasts, late erythroblasts, normoblasts, reticulocytes before nucleus expulsion, reticulocytes, or erythrocytes, or any combinations thereof. In particular embodiments, the target cell or target cells may comprise HSCs and/or HSPCs. In particular embodiments, the target cell or target cells may comprise cells that are CD34⁺.

### Target Genes

In one aspect, a target gene according to the present disclosure may be a gene associated with a target disease. In some embodiments, a target gene according to the present disclosure may be edited via any appropriate technique. In some embodiments, the expression (e.g., protein and/or mRNA level) of a target gene according to the present disclosure may be modified via any appropriate technique.

In some embodiments, the target gene may be a SCD-associated gene including a regulatory element thereof, e.g., a region of a promoter or enhancer of such a gene.

In some embodiments, the target gene may comprise a gene encoding a hemoglobin component such as beta-globin (the HBB gene) and/or a regulatory element thereof, e.g., a region of a promoter or enhancer of HBB. The generic sequence of human HBB may comprise the nucleic acid sequence corresponding to the nucleotide positions 5225464 to 5227071 of chromosome 11 (according to Gene Assembly GRCh38.p13). In some embodiments, the HBB gene may be the HbS variant of HBB, the causative gene of SCD. The HbS variant of HBB (coding strand) may comprise the nucleic acid sequence of SEQ ID NO: 21, which encodes the HbS variant of beta-globin having the amino acid sequence of SEQ ID NO: 2. In some embodiments, the HBB gene may be the HbC variant of HBB, which in the homozygous state can cause mild chronic hemolysis, splenomegaly, and jaundice. The HbC variant of HBB (coding strand) may comprise the nucleic acid sequence of SEQ ID NO: 31, which encodes the HbC variant of beta-globin having the amino acid sequence of SEQ ID NO: 3. In some embodiments, the target gene may comprise a gene encoding a gene product (e.g., transcription factor) that directly or indirectly regulates or a DNA region (e.g., promoter, enhancer, transcription factor-binding site) that directly or indirectly regulates the expression of the HBB gene.

In some embodiments, the target gene may comprise a gene encoding a gene product that regulates hemoglobin switching and/or erythropoiesis.

In some embodiments, the target gene may comprise a gene encoding BAF chromatin remodeling complex subunit BCL11A (the BCL11A gene) and/or a regulatory element thereof, e.g., a region of a promoter or enhancer of BCL11A. The generic sequence of human BCL11A may comprise the nucleic acid sequence corresponding to the nucleotide positions 60450520 to 60/553,654 of chromosome 2 (according to Gene Assembly GRCh38.p13), which encodes the amino acid sequence of SEQ ID NO: 6. In some embodiments, the target gene may comprise a gene encoding a gene product (e.g., transcription factor) that directly or indirectly regulates or a DNA region (e.g., promoter, enhancer, transcription factor-binding site) that directly or indirectly regulates the expression of the BCL11A gene.

In some embodiments, the target gene may comprise a gene encoding Kruppel like factor 1 (the KLF1 gene) and/or a regulatory element thereof, e.g., a region of a promoter or enhancer of KLF1. The generic sequence of human KLF1 may comprise the nucleic acid sequence corresponding to the nucleotide positions 12884422 to 12887201 of chromosome 19 (according to Gene Assembly GRCh38.p13), which encodes the amino acid sequence of SEQ ID NO: 7. In some embodiments, the target gene may comprise a gene encoding a gene product (e.g., transcription factor) that directly or indirectly regulates or a DNA region (e.g., promoter, enhancer, transcription factor-binding site) that directly or indirectly regulates the expression of the KLF1 gene.

In some embodiments, the target gene may comprise a gene encoding human hemoglobin subunit gamma 1 (the HBG1 gene) and/or a regulatory element thereof, e.g., a region of a promoter or enhancer of HBG1. The generic sequence of human HBG1 may comprise the nucleic acid sequence corresponding to the nucleotide positions 5248269 to 5249857 of Chromosome 11 (according to Gene Assembly GRCh38.p14), which encodes the amino acid sequence of SEQ ID NO: 8. In some embodiments, the target gene may comprise a gene encoding a gene product (e.g., transcription factor) that directly or indirectly regulates or a DNA region (e.g., promoter, enhancer, transcription factor-binding site) that directly or indirectly regulates the expression of the HBG1 gene, preferably the BCL11A-binding site in the promoter.

In some embodiments, the target gene may comprise a gene encoding human hemoglobin subunit gamma 2 (the HBG2 gene) and/or a regulatory element thereof, e.g., a region of a promoter or enhancer of HBG2. The generic sequence of human HBG2 may comprise the nucleic acid sequence corresponding to the nucleotide positions 5253188 to 5254781 of Chromosome 11 (according to Gene Assembly GRCh38.p14), which encodes the amino acid sequence of SEQ ID NO: 9. In some embodiments, the target gene may comprise a gene encoding a gene product (e.g., transcription factor) that directly or indirectly regulates or a DNA region (e.g., promoter, enhancer, transcription factor-binding site) that directly or indirectly regulates the expression of the HBG1 gene, preferably the BCL11A-binding site in the promoter.

In some embodiments, the target gene may be SOX6, GATA1, NF-E4 (or NFE4), COUP-TF, NR2C1 (also known as TR2), NR2C2 (also known as TR4), genes encoding members of the MBD2 protein complex, IKZF1 (also known as Ikaros), genes encoding other members of PYR complex (CHD4, HDAC2, RBBP7, SM4RCB1, SM4RCC1, SM4RCC2, SM4RCD1, and SMARCE1), or BRG1, or a gene that directly or indirectly modulate the expression thereof, or any combination thereof.

### Target Sequences

In one aspect, any parts of a target gene according to the present disclosure may be targeted (i.e., may be a target sequence), and the target sequence may be any parts of the sequence of the coding (sense) strand or the non-coding (antisense) strand of the gene or its transcript (including pre- and post-splicing sequences), any parts of the sequence of the coding region or non-coding region of the gene or its transcripts, or any parts of the sequence of the polynucleotide regions regulating the expression of the gene (e.g., promoter region, enhancer region, transcription factor-binding site).

For example, when the target gene is the HbS variant of human HBB, in some embodiments, the target sequence may be any parts of the nucleic acid sequence corresponding to the nucleotide positions 5225464 to 5227071 of chromosome 11 (according to Gene Assembly GRCh38.p13) or its transcript (including pre- and post-splicing sequences). In some embodiments, the target sequence may be any parts of the nucleic acid sequence of SEQ ID NO: 21. In some embodiments, the target sequence may be any parts of the nucleic acid sequence of SEQ ID NO: 22. In some embodiments, the target sequence may be any parts of the nucleic acid sequence of SEQ ID NO: 23. In certain embodiments, the target sequence may be the nucleic acid sequence of SEQ ID NO: 44, 46, or 24 or a variant thereof.

For example, when the target gene is human BCL11A, in some embodiments, the target sequence may be any parts of the nucleic acid sequence corresponding to the nucleotide positions 60450520 to 60/553,654 of chromosome 2 (according to Gene Assembly GRCh38.p13) or its transcript (including pre- and post-splicing sequences). In certain embodiments, the target sequence may be within or may overlap with intron 2 of human BCL11A, preferably within or overlapping with the erythroid-enhancer region (EER) therein. In particular embodiments, the target sequence may be the nucleic acid sequence of SEQ ID NO: 64, 68, or 66 or a variant thereof.

For example, when the target gene is human KLF1, in some embodiments, the target sequence may be any parts of the nucleic acid sequence corresponding to the nucleotide positions 12884422 to 12887201 of chromosome 19 (according to Gene Assembly GRCh38.p13) or its transcript (including pre- and post-splicing sequences). In certain embodiments, the target sequence may be the nucleic acid sequence of SEQ ID NO: 74 or 76 or a variant thereof.

For example, when the target gene is human HBG1, in some embodiments, the target sequence may be any parts of the nucleic acid sequence corresponding to the nucleotide positions 5248269 to 5249857 of Chromosome 11 (according to Gene Assembly GRCh38.p14) or its transcript (including pre- and post-splicing sequences). In certain embodiments, the target sequence may be the nucleic acid sequence of SEQ ID NO: 84 or a variant thereof.

For example, when the target gene is human HBG2, in some embodiments, the target sequence may be any parts of the nucleic acid sequence corresponding to the nucleotide positions 5253188 to 5254781 of Chromosome 11 (according to Gene Assembly GRCh38.p14) or its transcript (including pre- and post-splicing sequences). In certain embodiments, the target sequence may be the nucleic acid sequence of SEQ ID NO: 84 or a variant thereof.

### Gene Editing

### Gene Editing Tools

In one aspect, a target gene according to the present disclosure may be edited in vivo. Gene editing may be effected by any appropriate techniques. In some embodiments, gene editing may be mediated by a nuclease, endonuclease, meganuclease, zinc finger nuclease (ZFN), transcription activator-like nuclease (TALEN), or Cas nuclease. In some embodiments, gene editing may result in at least one nucleic acid insertion, deletion, or replacement (e.g., resulting in a nonsense, missense, or silent mutation) in the target gene, such as SCD-associated gene, such as HBB, BCL11A, or KLF1.

### Cas Nucleases

When the CRISPR/Cas system is used for gene editing, any appropriate Cas nucleases may mediate gene editing. In some embodiments, a Cas nuclease (as a protein) or a Cas nuclease-encoding polynucleotide (e.g., DNA or RNA) may be used. In some embodiments, the Cas nuclease may be Cas 9, Cas3, Cas8a2, Cas8b, Cas8c, Cas10, Csx11, Cas12, Cas12a or Cpf1, Cas13, Cas13a, C2c1, C2c3, or C2c2. In some embodiments, the Cas nuclease may be a class 2 Cas nuclease. In some embodiments, the Cas nuclease may be a type V or type VI Cas nuclease. In certain embodiments, the Cas nuclease is Cas9. In certain embodiments, the Cas9 may be Cas9 of Streptococcus pyogenes (SpCas9), Staphylococcus aureus Cas9 (SaCas9), Streptococcus thermophilus (StCas9), Neisseria meningitidis (NmCas9), Francisella novicida (FnCas9), Campylobacter jejuni (CjCas9), Streptococcus canis (ScCas9), Staphylococcus auricularis (SauriCas9), or any engineered variants thereof, including SaCas9-HF, SpCas9-HF1, KKHSaCas9, eSpCas9, HypaCas9, FokI-Fused dCas9, xCas9, SpRY (variant of SpCas9), or SpG (variant of SpCas9). Cas nuclease of different bacterial origins often recognize different PAM sequences and/or different cleavage accuracy or specificity. In some cases, the type of Cas nuclease to use may be selected based on the presence or absence or a certain PAM sequence in the target gene.

### Guide RNA

When the CRISPR/Cas system is used for gene editing, the gRNA may be designed based on the sequence of the target gene and the PAM sequence recognized by the Cas nuclease to be used. When Cas9 of Streptococcus pyogenes is used, the target-complementary sequence of a gRNA may be designed, for example, as the 20 (or alternatively about 17-24) nucleotides immediately upstream (the 5′-side) of any of the 5′-NGG-3′ (N may be any nucleic acid) PAM sites present in the target gene (the coding strand or non-coding strand). A desired target-complementary sequence may be selected from all possible sequences, for example based on, the proximity to the desired editing position, the G-C content (e.g., for example in the range of about 40-80%), self-complementarity, the potential editing efficiency, and/or the potential off-target effects. Non-limiting tools for selecting a desired target-complementary sequence include https://chopchop.cbu.nib.no/.

Exemplary target-complementary sequences of a gRNA for targeting human HBB (any variants) include but are not limited to SEQ ID NOS: 25, 45, 47, and 49, which targets the target sequence of SEQ ID NOS: 24, 44, 46, and 48, respectively, or a variant thereof.

Exemplary target-complementary sequences of a gRNA for targeting the HbS variant of human HBB include but are not limited to SEQ ID NOS: 25, which may target the target sequence of SEQ ID NOS: 24.

Exemplary target-complementary sequences of a gRNA for targeting human BCL11A include but are not limited to SEQ ID NOS: 65, 69, and 67, which may target the target sequence of SEQ ID NOS: 64, 68, and 66, respectively.

Exemplary target-complementary sequences of a gRNA for targeting human KLF1 include but are not limited to SEQ ID NOS: 75 and 77, which may target the target sequence of SEQ ID NOS: 64 and 76, respectively.

Exemplary target-complementary sequences of a gRNA for targeting human HBG1 and/or HBG2 include but are not limited to SEQ ID NO: 85, which targets the target sequence of SEQ ID NOS: 84 or a variant thereof.

gRNA Modifications

In some embodiments, a gRNA according to the present disclosure may comprise one or more modifications. In some embodiments, the modification may be selected from the group consisting of: 2′-O—C1-4alkyl such as 2′-O-methyl (2′-OMe), 2′-deoxy (2′-H), 2′-O—C1-3alkyl-O—C1-3alkyl such as 2′-methoxyethyl (2′-MOE), 2′-fluoro (2′-F), 2′-amino (2′-NH2), 2′-arabinosyl (2′-arabino) nucleotide, 2′-F-arabinosyl (2′-F-arabino) nucleotide, 2′-locked nucleic acid (LNA) nucleotide, 2′-unlocked nucleic acid (ULNA) nucleotide, a sugar in 1 form (1-sugar), and 4′-thioribosyl nucleotide. In some embodiments, the modification is an internucleotide linkage modification selected from the group consisting of: phosphorothioate, phosphonocarboxylate, thiophosphonocarboxylate, alkylphosphonate, and phosphorodithioate. In some embodiments, the modification is selected from the group consisting of: 2-thiouracil (2-thioU), 2-thiocytosine (2-thioC), 4-thiouracil (4-thioU), 6-thioguanine (6-thioG), 2-aminoadenine (2-aminoA), 2-aminopurine, pseudouracil, hypoxanthine, 7-deazaguanine, 7-deaza-8-azaguanine, 7-deazaadenine, 7-deaza-8-azaadenine, 5-methylcytosine (5-methylC), 5-methyluracil (5-methylU), 5-hydroxymethylcytosine, 5-hydroxymethyluracil, 5,6-dehydrouracil, 5-propynylcytosine, 5-propynyluracil, 5-ethynylcytosine, 5-ethynyluracil, 5-allyluracil (5-allylU), 5-allylcytosine (5-allylC), 5-aminoallyluracil (5-aminoallylU), 5-aminoallyl-cytosine (5-aminoallylC), an abasic nucleotide, Z base, P base, Unstructured Nucleic Acid (UNA), isoguanine (isoG), isocytosine (isoC), and 5-methyl-2-pyrimidine. In particular embodiments, a gRNA may comprise (i-1) 2-O-methylation further optionally at first three and last three bases and/or (i-2) one or more 3′ phosphorothioate bonds, further optionally between first three and last two bases.

### DNA Repair Templates

In one aspect, when the CRISPR/Cas system is used for gene editing and when a gene knock-in or gene sequence correction is desired, a DNA repair template (or simply referred to as a repair template) comprising a desired mutation or sequence may further be provided so that a gene knock-in or gene sequence correction is effected based on the template sequence via the cells' endogenous DNA repair mechanisms. When Cas9 is used, Cas9 provides a double-strand break (DSB) in the target gene between the third and fourth nucleotides upstream (5′ side) of the 5′-NGG-3′ and if a repair template is provided, homology-directed repair (HDR) will take place. In some embodiments, a repair template may be a single-stranded (ssODN) or double-stranded.

In some embodiments, a repair template comprises or consists of a 5′ homology arm, a central region, and a 3′ homology arm. In some embodiments, a repair template may be approximately centered with respect to the DSB position. In some embodiments, the DSB position may be in the central region. In some embodiments, a homology arm may be less than about 30, about 25, about 20, about 20, about 15, or about 10 nt away from the DSB position. In particular embodiments, a homology arm may be about 10, about 8, about 6, about 4, about 3, about 2, or about 1 nt away from the DSB position.

In some embodiments, a DNA repair template may comprise a total length of approximately 40-5000 nt. In some embodiments, the total length may be about 40-2000 nt, about 40-1000 nt, about 40-500 nt, about 40-200 nt, about 80-160 nt, about 100-140 nt, about 110-130 nt, or about 120 nt.

Any appropriate size of a homology arm may be used. In some embodiments, the 5′ and 3′ homology arms may have the same or similar nucleotide lengths (e.g., 1, 2, 3, 4, 5, 6, 7, 8, 9, or 10 nt difference). In some embodiments, the 5′ and 3′ homology arms may significantly differ in length. In some embodiments, the size of a homology arm may be approximately 20-2500 nucleotides (nt), about 20-1000 nt, about 20-500 nt, or about 20-100 nt. In particular embodiments, the size of a homology arm may be about 40-80 nt, about 50-70 nt, or about 60 nt. In particular embodiments, the size of each homology arm may be about 40-80 nt, about 50-70 nt, or about 60 nt. In some embodiments, 5′ and/or 3′ homology anns may be 100% complementary to the corresponding sequence in the original DNA sequence before gene editing or may have one or more (a few, for example, 2, 3, 4, or 5) mutations (e.g., silent mutation) relative to the corresponding sequence in the original DNA sequence before gene editing.

In some embodiments, a repair template may have one or more mutations at one or more of the PAM positions. In some embodiments, such a mutation(s) helps prevent or reduce Cas-mediated cleavage of the repair template itself or of the DNA molecule post HDR. In case of a ssODN, such a mutation may be within the PAM bases or the reverse (or antisense) bases, i.e., the opposite strand) and/or at one or more of the 5′-neighbouring bases of the PAM (or the 3′-neighbouring bases of the reverse (or antisense) sequences corresponding to the PAM). In some embodiments, a ssODN may comprise complementarity to the gRNA strand.

In some embodiments when the target gene is the HbS variant of HBB, a knock-in of wild type HBB may be desired.

In some embodiments, a ssODN encoding part of the wildtype beta-globin, or a ssODN comprising a sequence complementary thereto may be used. In certain embodiments, the part of the wildtype beta-globin may be encoded by the wildtype nucleic acid sequence. In certain embodiments, the part of the wildtype beta-globin may be encoded by a nucleic acid sequence comprising one or more mutations (e.g., silent mutation(s)) relative to the wildtype nucleic acid sequence.

In some embodiments, the ssODN may comprise a central region having the sequence of 5′-CTCA-3′, 5′-TTCA-3′, 5′-CTCT-3′, 5′-TTCT-3′, 5′-CTCC-3′, 5′-TTCC-3′, 5′-CTCG-3′, or 5′-TTCG-3′. Any of these central region sequences, once knocked-in, would correct the glutamate-to-valine (“E-to-V”) SCD-causing amino acid substitution back to the wild type glutamate.

In some embodiments, the ssODN may comprise a 5′ homology arm having the sequence of SEQ ID NO: 112. Alternatively, a 5′ homology arm may have part of the 3′ sequence of SEQ ID NO: 112. In some embodiments, a 5′ homology arm may have any length of 20 nt or longer counting from the 3′ end of SEQ ID NO: 112. In certain embodiments, a 5′ homology arm may have a length of at least 20, at least 30 (such as 39), at least the 40 (such as 49), or at least 50 (such as 50 or 59) nt counting from the 3′-end of SEQ ID NO: 112. In some embodiments, the 5′ homology arm may further comprise one or more mutations, preferably silent mutation(s), relative to such 5′ homology arm sequences.

In some embodiments, the ssODN may comprise a 3′ homology arm having the sequence of SEQ ID NO: 122. Alternatively, a 3′ homology arm may have part of the 5′ sequence of SEQ ID NO: 122. In some embodiments, a 3′ homology arm may have any length of 20 nt or longer counting from the 5′ end of SEQ ID NO: 122. In certain embodiments, a 3′ homology arm may have a length of at least 20, at least 30 (such as 37), at least the 40 (such as 47), or at least 50 (such as 57) nt counting from the 5′-end of SEQ ID NO: 122. In some embodiments, the 3′ homology arm may further comprise one or more mutations, preferably silent mutation(s), relative to such 3′ homology arm sequences.

In particular embodiments, the ssODN may comprise, essentially consist of or consist of the sequence of any one of SEQ ID NOS: 101-108.

In further embodiments, the ssODN may comprise a sequence complementary to any of the sequence of ssODNs for correcting back to a wildtype beta globin-encoding DNA sequence described herein.

In some embodiments, when a DNA repair template, a double-stranded DNA template may also be used instead. In such a case, one of the strands of the template may comprise the same sequence as a desired ssODN and the other strand have a sequence complementary thereto.

### Gene Expression Alteration

### Expression Alteration Tools

In one aspect, the expression of a target gene according to the present disclosure maybe altered in vivo. Gene expression may be altered by any appropriate techniques. In some embodiments, gene expression may be altered via a nucleic acid molecule, such as but not limited to: a RNA (single or double stranded), a siRNA, a shRNA, a miRNA, a mRNA, a DNA (single or double stranded), a plasmid DNA, a cDNA, and/or a locked nucleic acid. Gene expression alteration may be effected alone or in combination with gene editing described herein.

When reduced expression of a target gene is desired: In some embodiments, the target gene expression may be altered via RNA interference. For example, in some embodiments, an siRNA or shRNA specific to the transcript sequence of a target gene may be used. In some embodiments, a miRNA which negatively modulates the target gene expression may be introduced. In further embodiments, an RNA or DNA molecule encoding a gene that negatively modulates the target gene expression (e.g., repressor of the target gene) may be introduced, via transduction or transfection.

When increased expression of a target gene is desired: In some embodiments, the target gene expression may be altered via forced expression of the gene, via transduction or transfection of a nucleic acid molecule encoding the gene. In some embodiments, the target gene expression may be altered via transduction or transfection of a nucleic acid molecule encoding a gene that positively modulates the target gene expression (e.g., a transcription factor). In further embodiments, a negative regulator of the target gene may be silenced via RNA interference, using e.g., an siRNA or shRNA.

### Delivery Vehicles

### Vehicle Type

In one aspect, any components that may be used for effecting gene editing and/or gene expression alteration as described herein may be carried into as a cargo (or cargoes) into a cell by a delivery vehicle. Such a delivery vehicle may be a transfection competent vehicle (TCV).

### Lipid-Based TCVs

TCVs particularly used in the present disclosure include lipid-based TCVs. Compared to non-lipid-based TCVs such as viral vectors, lipid-based TCVs may have several advantages, e.g., less immunogenicity if needed, no random integration into the target cell genome.

### Ionizable Cationic Lipid

In some embodiments, a lipid-based TCV may comprise at least one ionizable cationic lipid.

In some embodiments, the at least one ionizable cationic lipid may comprise DODMA, DODAP, DLinDAP, KC2, MC3, DODAC, DDAB, DOTAP, DOTMA, DLinDMA, DLenDMA, DLin-C-DAP, DLin-DAC, DLin-MA, DLin-S-DMA, DLin-2-DMAP, DLin-TMA·Cl, DLin-TAR·Cl, DLin-MPZ, DLinAP, DOAP, DLin-EG-DMA, DLin-K-DMA, DLin-K-DMA or analogs thereof, ALNY-100, DOTMA, DOTAP·Cl, DC-Chol, DOSPA, DOGS”, DMRIE, or any combinations thereof. In particular embodiments, the at least one ionizable cationic lipid may comprise or consist of DODMA.

The amount of the at least one ionizable cationic lipid may be determined as appropriate. In some cases, the amount of the at least one ionizable cationic lipid to be used may be determined based on the type of cargo.

In some embodiments, the amount of ionizable cationic lipid(s) relative to the total amount of TCV components may be about 10 mol % to about 70 mol %. In some embodiments, the total amount of TCV components may be about 10 mol % to about 60 mol %, about 10 mol % to about 50 mol %, about 10 mol % to about 40 mol %, about 10 mol % to about 30 mol %, about 15 mol % to about 25 mol %, about 18 mol % to about 22 mol %, about 19 mol % to about 21 mol %, about 19.5 mol % to about 20.5 mol %, about 19.8 mol % to about 20.2 mol %, or about 20 mol %. In particular embodiments, for example when the cargo comprises a nucleic acid and a protein (or a RNP), the total amount of ionizable cationic lipid(s) relative to the total amount of TCV components may be about 20 mol %.

In a preferred embodiment, a lipid-based TCV according to the present disclosure comprises DODMA at 20 mol % relative to the total amount of TCV components.

In some embodiments, the amount of ionizable cationic lipid(s) relative to the total amount of TCV components may be about 10 mol % to about 70 mol %, about 20 mol % to about 70 mol %, about 30 mol % to about 70 mol %, about 40 mol % to about 70 mol %, about 40 mol % to about 60 mol %, about 45 mol % to about 55 mol %, about 48 mol % to about 52 mol %, about 49 mol % to about 51 mol %, about 49.5 mol % to about 50.5 mol %, about 49.8 mol % to about 50.2 mol %, or about 50 mol %. In particular embodiments, for example when the cargo comprises a nucleic acid such as a siRNA, sihRNA or miRNA or a RNA or DNA vector, the total amount of ionizable cationic lipid(s) relative to the total amount of TCV components may be about 50 mol %.

In a preferred embodiment, a lipid-based TCV according to the present disclosure comprises DODMA at 50 mol % relative to the total amount of TCV components.

### Helper Lipid

In some embodiments, a lipid-based TCV may comprise at least one helper lipid in addition to the at least one ionizable cationic lipid. In some embodiments, the at least one helper lipid may comprise DOPE, DSPC, DOPC, DPPC, DOPG, DPPG, POPC, POPE, DOPE-mal, DPPE, DMPE, DSPE, 16-O-monomethyl PE, 16-O-dimethyl PE, 18-1-trans PE, SOPE, or any combinations thereof. In particular embodiments, the at least one helper lipid may comprise or consist of DOPE. In some cases, the at least one helper lipid to be used may be determined based on the stability of the TCV.

The amount of the at least one helper lipid may be determined as appropriate.

In some embodiments, the total amount of helper lipid(s) relative to the total amount of TCV components may be about 10 mol % to about 60 mol %. In some embodiments, the total amount of helper lipid(s) relative to the total amount of TCV components may be about 10 mol % to about 50 mol %, about 10 mol % to about 40 mol %, about 20 mol % to about 40 mol %, about 25 mol % to about 35 mol %, about 28 mol % to about 32 mol %, about 29 mol % to about 31 mol %, about 29.5 mol % to about 30.5 mol %, about 29.8 mol % to about 30.2 mol %, or about 30 mol %. In particular embodiments, the total amount of helper lipid(s) relative to the total amount of TCV components may be about 30 mol %.

In some embodiments, the total amount of helper lipid(s) relative to the total amount of TCV components may be about 20 mol % to about 60 mol %, about 30 mol % to about 50 mol %, about 35 mol % to about 45 mol %, about 38 mol % to about 42 mol %, about 39 mol % to about 41 mol %, about 39.5 mol % to about 40.5 mol %, about 39.8 mol % to about 40.2 mol %, or about 40 mol %. In particular embodiments, the total amount of helper lipid(s) relative to the total amount of TCV components may be about 40 mol %.

In a preferred embodiment, a lipid-based TCV according to the present disclosure comprises DOPE at 30 mol %. Such a TCV may be used, for example when the cargo comprises a nucleic acid and a protein (or a RNP).

### Phospholipid

In some embodiments, a lipid-based TCV may comprise at least one phospholipid in addition to the at least one ionizable cationic lipid. In some embodiments, the at least one phospholipid may comprise DSPC, DOPE, DPPC, DOPC, DMPC, PLPC, DAPC, PE, EPC, DLPC, DMPC, MPPC, PMPC, PSPC, DBPC, SPPC, DEPC, POPC, lysophosphatidyl choline, DSPE, DMPE, DPPE, POPE, lysophosphatidylethanolamine, or any combinations thereof. In particular embodiments, the at least one helper lipid may comprise or consist of DSPC.

In some embodiments, the amount of phospholipid(s) relative to the total amount of TCV components may be about 5 mol % to about 65 mol %, about 5 mol % to about 55 mol %, about 5 mol % to about 45 mol %, about 5 mol % to about 35 mol %, about 5 mol % to about 25 mol %, about 5 mol % to about 15 mol %, about 8 mol % to about 12 mol %, about 9 mol % to about 11 mol %, about 9.5 mol % to about 10.5 mol %, about 9.8 mol % to about 10.2 mol %, or about 10 mol %. In particular embodiments, the total amount of phospholipid(s) relative to the total amount of TCV components may be about 10 mol %.

In some embodiments, the total amount of phospholipid(s) relative to the total amount of TCV components may be about 5 mol % to about 65 mol %, about 15 mol % to about 65 mol %, about 25 mol % to about 55 mol %, about 35 mol % to about 45 mol %, about 38 mol % to about 42 mol %, about 39 mol % to about 41 mol %, about 39.5 mol % to about 40.5 mol %, about 39.8 mol % to about 40.2 mol %, or about 40 mol %. In particular embodiments, the total amount of phospholipid(s) relative to the total amount of TCV components may be about 40 mol %.

In a preferred embodiment, a lipid-based TCV according to the present disclosure comprises DSPC at 10 mol % relative to the total amount of TCV components. Such a TCV may be used, for example when the cargo comprises a nucleic acid molecule or nucleic acid and a protein (or a RNP complex).

### Cholesterol or Cholesterol Derivative

In some embodiments, a lipid-based TCV may comprise at least one cholesterol or cholesterol derivative in addition to the at least one ionizable cationic lipid. In some embodiments, the at least one cholesterol or cholesterol derivative may comprise cholesterol, DC-Chol, 1,4-bis(3-N-oleylamino-propyl)piperazine, ICE, or any combinations thereof. In particular embodiments, the at least one cholesterol or cholesterol derivative may comprise or consist of cholesterol.

In some embodiments, the amount of cholesterol and/or cholesterol derivative(s) relative to the total amount of TCV components may be about 20 mol % to about 60 mol %. some embodiments, the amount of cholesterol and/or cholesterol derivative(s) relative to the total amount of TCV components may be about 25 mol % to about 55 mol %, about 30 mol % to about 50 mol %, about 35 mol % to about 45 mol %, about 38 mol % to about 42 mol %, about 39 mol % to about 41 mol %, about 39.5 mol % to about 40.5 mol %, about 39.8 mol % to about 40.2 mol %, or about 40 mol %, or about 39%. In particular embodiments, the total amount of cholesterol and/or cholesterol derivative(s) relative to the total amount of TCV components may be about 40 mol % or about 39 mol %.

In a preferred embodiment, a lipid-based TCV according to the present disclosure comprises cholesterol at 40 mol % or 39 mol % relative to the total amount of TCV components. Such a TCV may be used, for example when the cargo comprises a nucleic acid molecule or a nucleic acid and a protein (or a RNP complex).

### PEG-Lipid

In some embodiments, a lipid-based TCV may comprise at least one PEG-lipid in addition to the at least one ionizable cationic lipid. In some embodiments, the at least one PEG-lipid may comprise PEG-DMG (e.g., (Avanti® Polar Lipids (Birmingham, AL)), PEG-phosphatidylethanolamine and phosphatidic acid, PEG-ceramide conjugates (e.g., PEG-CerC14 or PEG-CerC20), PEG-modified dialkylamines, PEG-modified 1,2-diacyloxypropan-3-amines, or any combinations thereof. In particular embodiments, the at least one PEG-lipid may comprise or consist of PEG-DMG.

In some embodiments, the amount of PEG and/or PEG-lipid(s) relative to the total amount of TCV components may be about 0.1 mol % to about 5 mol %, 0.1 mol % to about 4 mol %, 0.1 mol % to about 3 mol %, 0.1 mol % to about 2 mol %, 0.5 mol % to about 1.5 mol %, 0.8 mol % to about 1.2 mol %, 0.9 mol % to about 1.1 mol %, or about 1 mol %. In particular embodiments, the total amount of PEG-lipid(s) relative to the total amount of TCV components may be about 1 mol %.

In a preferred embodiment, a lipid-based TCV according to the present disclosure comprises PEG-DMG at 1 mol % relative to the total amount of TCV components.

In a preferred embodiment, a lipid-based TCV according to the present disclosure comprises DODMA at 20 mol %, DOPE at 30 mol %, DSPC at 10 mol %, and cholesterol at 40 mol % relative to the total amount of TCV components. Such a TCV may be used, for example when the cargo comprises a nucleic acid and a protein (or a RNP complex).

In another preferred embodiment, a lipid-based TCV according to the present disclosure comprises DODMA at 50 mol %, DSPC at 10 mol %, cholesterol at 39 mol %, PEG-DMG at 1 mol % relative to the total amount of TCV components. Such a TCV may be used, for example when the cargo comprises a nucleic acid molecule.

### Surface Modulation

In some embodiments, a TCV according to the present disclosure may comprise a targeting moiety. The targeting moiety to be incorporated may be determined based on the target cell type, so that the TCV may preferentially carry its cargo into the target cells.

The targeting moiety may be any type of materials that allows for specific or preferential binding to a target cell. In some embodiments, the targeting molecule may be a protein (e.g., an antibody or an antibody fragment), a peptide, a nucleic acid (e.g., aptamer), a small molecule, or another material (e.g., a vitamin or a carbohydrate).

In some embodiments, the targeting moiety may be designed to be specific for HSCs, HSPCs, MPPs, CMPs, MEPs, HPCs, erythroid progenitors (e.g., BFU-Es, CFU-Es), proerythroblasts, erythroblasts (basophilic erythroblasts, early erythroblasts (e.g., type I, type II), polychromatic erythroblasts, intermediate erythroblasts, acidophilic erythroblasts, late erythroblasts, normoblasts, reticulocytes (before nucleus expulsion), or any combinations thereof.

In particular embodiments, the targeting moiety may be designed to be specific for HSCs and/or HSPCs. In certain embodiments, the targeting moiety may specifically or preferentially bind to CD34, which is present on HSCs and/or HSPCs. In certain embodiments, the targeting moiety may be an antibody or an antibody fragment specific to CD34.

### TCV Size

In some embodiments, the size of TCVs may be determined by any appropriate techniques. Non-limiting examples of measurement methods include dynamic light chattering, binding assays, surface plasmon resonance (SPR), static image analysis, and dynamic image analysis. An appropriate measurement technique may be selected based on the accuracy and the approximate size range the technique is optimal for.

In some embodiments, the size of the TCV before encapsulation of the at least one cargo may be in a range of about 3 nm to about 240 nm, about 6 nm to about 160 nm, about 9 nm to about 80 nm, or about 20 nm to about 40 nm, at pH of about 4. In particular embodiments, the size of the TCV before encapsulation of the at least one cargo may be in a range of about 9 nm to about 80 nm at pH of about 4.

### Pharmaceutical Compositions

### Compositions for Gene Editing

In one aspect, a pharmaceutical composition according to the present disclosure comprises at least one cargo which is capable of effecting gene editing. In some embodiments, the gene editing may a SCD-associated gene. In some embodiments, the gene editing takes place in vivo. For example, the gene editing may take place in the bone marrow (in a cell in the bone marrow). Alternatively, the gene editing may take place in the peripheral circulation (in a cell in the peripheral circulation).

In some embodiments, a pharmaceutical composition which may be used for effecting gene editing may comprise a Cas nuclease and a gRNA. Non-limiting examples of Cas proteins and gRNAs that may be contained in a pharmaceutical composition are as described herein. In some embodiments, the Cas nuclease and the gRNA contained in a pharmaceutical composition may be forming a complex, i.e., RNP.

In particular embodiments, the Cas nuclease contained in a pharmaceutical composition may be a Cas9 nuclease. In a particular embodiment, the Cas9 nuclease may be Cas9 of Streptococcus pyogenes (SpCas9). In particular embodiments, the gRNA contained in a pharmaceutical composition may comprise a target-complementary sequence of any one of SEQ ID NOS: 25, 65, 67, 75, and 77.

In some embodiments, when a gene knock-in and/or gene correction is desired, a pharmaceutical composition may further comprise a repair DNA template. In particular embodiments, the repair DNA template may be a ssODN. In some embodiments, the repair DNA template may be encapsulated in the same TCV as the Cas nuclease and gRNA (or RNP) or in a separate TCV.

In some embodiments, when correction in the HBB gene back to a gene encoding a wildtype beta-globin is desired, the repair DNA template may be any of the repair DNA template for HBB described herein. In particular embodiments, the repair DNA template may be a ssODN comprising the nucleic acid sequence of any one of SEQ ID NOS: 101-108, or a sequence fully complementary thereto. In particular embodiments, the repair DNA template may be a ssODN comprising the nucleic acid sequence of SEQ ID NO: 101 or 102, or a sequence fully complementary thereto.

### Compositions for Gene Expression Alteration

In one aspect, a pharmaceutical composition according to the present disclosure comprises at least one cargo which is capable of effecting gene expression alteration. In some embodiments, the expression a SCD-associated gene may be altered, e.g., upregulated or downregulated, or increased or decreased. In some embodiments, the gene expression alteration takes place in vivo. For example, the gene expression alteration (or modification) may take place in the bone marrow (in a cell in the bone marrow). Alternatively, the gene expression alteration (or modification) may take place in the peripheral circulation (in a cell in the peripheral circulation).

In some embodiments, a pharmaceutical composition which may be used for effecting gene expression alteration (or modification) may comprise a nucleic acid molecule. Any of the nucleic acid molecules that mediate gene expression alteration (or modification) disclosed herein may be contained in a pharmaceutical composition.

### Cargo Combinations

In some embodiments, a pharmaceutical composition according to the present disclosure may comprise a TCV comprising (i) at least one cargo which is capable of effecting gene editing and (ii) at least one cargo capable of effecting gene expression alteration. In some embodiments, a pharmaceutical composition according to the present disclosure may comprise (i) a TCV comprising at least one cargo which is capable of effecting gene editing and (ii) a separate TCV comprising at least one cargo capable of effecting gene expression alteration.

In some embodiments, a pharmaceutical composition according to the present disclosure may comprise a TCV comprising (i) at least one first cargo which is capable of effecting first gene editing and (ii) at least one second cargo capable of effecting second gene editing. In some embodiments, a pharmaceutical composition according to the present disclosure may comprise (i) a first TCV comprising at least one first cargo which is capable of effecting first gene editing and (ii) a second TCV comprising at least one second cargo capable of effecting second gene editing.

In some embodiments, a pharmaceutical composition according to the present disclosure may comprise a TCV comprising (i) at least one first cargo which is capable of effecting first gene expression alteration and (ii) at least one second cargo capable of effecting second gene expression alteration. In some embodiments, a pharmaceutical composition according to the present disclosure may comprise (i) a first TCV comprising at least one first cargo which is capable of effecting gene expression alteration and (ii) a second TCV comprising at least one second cargo capable of effecting second gene expression alteration.

### Organic Solvents and Detergents

In some embodiments, one characteristic of a pharmaceutical composition is that the composition is substantially, essentially, or entirely free of destabilizing agents, and/or contains significantly lower amounts of destabilizing agents compared to other pharmaceutical compositions comprising a similar type of TCVs.

In some embodiments, one characteristic of a pharmaceutical composition is that the composition is substantially, essentially, or entirely free of organic solvents and detergents, and/or contains significantly lower amounts of organic solvents and detergents compared to other pharmaceutical compositions comprising a similar type of TCVs.

In some embodiments, one characteristic of a pharmaceutical composition is that the composition is substantially, essentially, or entirely free of ethanol, methanol, isopropanol, tetrahydrofuran (THF), dimethyl sulfoxide (DMSO), dimethyl formamide (DMF), and acetonitrile (ACN), and/or contains significantly lower amounts of ethanol, methanol, isopropanol, THF, DMSO, DMF, and ACN, compared to other pharmaceutical compositions comprising a similar type of TCVs.

In particular embodiments, the pharmaceutical composition may be entirely free of methanol, isopropanol, THF, DMSO, DMF, and ACN.

With regard to ethanol, in some embodiments, the pharmaceutical composition may be substantially free of ethanol, which may mean that the ethanol concentration is 5% (v/v) or below. In particular embodiments, the pharmaceutical composition may be essentially free of ethanol, which may mean that the ethanol concentration is 0.5% (v/v) or below.

In a particular embodiment, the pharmaceutical composition may be entirely free of ethanol, methanol, isopropanol, THF, DMSO, DMF, and ACN.

### Additional Components

In one aspect, a pharmaceutical composition may comprise an additional component in addition to at least one cargo for effecting gene editing or gene expression alteration.

### Stem Cell Mobilization Agents

In some embodiments, the pharmaceutical composition may further comprise at least one stem cell mobilization agent. In some embodiments, the at least one stem cell mobilization agent may be contained in the composition outside the TCVs comprising the at least one cargo for effecting gene editing or gene expression alteration.

In some embodiments, a composition comprising a at least one stem cell mobilization agent may be suited for injection into the peripheral circulation, e.g., IV injection. In such an embodiment, the at least one stem cell mobilization agent may promote exit of stem cells (e.g., HSPs and/or HSPCs) into the circulation, and this may help TCVs efficiently enter the stem cells in the circulation. In some embodiments, such a composition may be suited for continuous injection for a period of time (e.g., 3 hours, 6 hours, 12 hours, 18 hours, 24 hours) in some instances multiple times. Without wishing to be bound by theory, the continuous and/or multiple loading of at least one stem cell mobilization may provide relatively sustained levels of stem cells in circulation that TCVs may enter.

In some embodiments, the at least one stem cell mobilization agent may comprise G-CSF (filgrastim), GM-CSF, Plerixafor, SCF, CXCR4 antagonists (e.g., POL6326, BKT-140, TG-0054), CXCL12 neutralizers (e.g., NOX-A12), Sphingosine-1-phosphate (SIP) antagonists (e.g., SEW2871), VCAM/VLA-4 inhibitors (e.g., BIO 5192), parathyroid hormone, protease inhibitors (e.g., Bortezomib), Groβ (e.g., SB-251353), hypoxia inducible factor (HIF) stabilizers (e.g., FG-4497), or any combinations thereof.

In some embodiments, the at least one stem cell mobilization agents may comprise G-CSF (filgrastim). In some embodiments, the at least one stem cell mobilization agents may comprise Plerixafor. In some embodiments, the at least one stem cell mobilization agents may comprise G-CSF (filgrastim) and Plerixafor.

### Erythropoiesis Stimulating Agents

In some embodiments, the pharmaceutical composition may further comprise at least one erythropoiesis stimulating agent. In some embodiments, the at least one erythropoiesis stimulating agent may be contained in the composition inside or outside a TCV, and if inside, the TCV may be the TCV comprising the at least one cargo for effecting gene editing or gene expression alteration or a separate TCV. In some embodiments, if the agent is an extracellular factor (e.g., growth factor, cytokine), the agent may be contained outside a RCV. In some embodiments, if the agent is an intracellular factor (e.g., transcription factor) or an agent that requires an intracellular machinery (e.g., a nucleic acid that needs to be transcribed and/or translated to function).

In some embodiments, the at least one erythropoiesis stimulating agent may help more of the HSCs and/or HSPCs the TCV comprising the at least one cargo for effecting gene editing or gene expression alteration entered ultimately differentiate into red blood cells. In some embodiments, the at least one erythropoiesis stimulating agent may help more of the cells at the early stages of erythropoiesis (e.g., MMPs, CMPs, MEPs) the TCV comprising the at least one cargo for effecting gene editing or gene expression alteration entered ultimately differentiate into red blood cells.

In some embodiments, the at least one erythropoiesis stimulating agent may comprise SCF, GM-CSF, interleukin-3 (IL-3), interleukin-9 (IL-9), erythropoietin (EPO), TGF-beta, growth differentiating factor 11 (GDF11), Activin A, Transferrin (Tf), ferritin, ferroportin, hepcidin, vitamin B12, folic acid, copper, or any combinations thereof. In some embodiments, such an agent may be encapsulated outside a TCV.

In some embodiments, the at least one erythropoiesis stimulating agent may comprise an agent selected from the group consisting of GATA-1, STAT5A, STAT5B, MCL-1, BCL-xL, HSP70, or any combinations thereof, or a RNA or DNA encoding thereof. In some embodiments, such an agent may be encapsulated in the TCV encapsulating the at least one cargo for effecting gene editing or gene expression alteration or in a separate TCV.

In some embodiments, the at least one erythropoiesis stimulating agent may comprise an inhibitor or silencer of a negative regulator of erythropoiesis. In some embodiments, such a negative regulator may be inhibin, TGF-beta, BID (a member of the BCL-2 family), Fas ligand, Fas, caspase, or any combinations thereof. In some embodiments, the agent may be encapsulated in the TCV encapsulating said at least one cargo for effecting gene editing or gene expression alteration or in a different TCV. Alternatively, in some embodiments, the agent may be encapsulated outside a TCV.

Additional exemplary erythropoiesis stimulating agents include but are not limited to engineered EPOs (such as Darbepoetin alfa, AMG 205, AMG 114, Pegzyrepoetin alfa, or MK-2578), EPO mimetics (such as EMP1, CNTO 528, CNTO 530, or Peginesatide), or anti-EPO receptor agonistic antibodies. Non-limiting examples of such agents are reviewed in detail in Sinclair. Biologics. 2013; 7:161-74. Epub 2013 Jul. 3.

### Agents for Treating SCD

In some embodiments, the pharmaceutical composition may further comprise at least another agent for treating SCD. In some embodiments, the at least another agent may be hydroxyurea, L-glutamine oral powder, crizanlizumab, a general pain medication, voxelotor, or any combination thereof.

### Manufacturing

### TCVs

As described above, one characteristic of a pharmaceutical composition according to some embodiments may be that the composition substantially, essentially, or entirely lacks organic solvents and detergents, which may help improve the stability and/or integrity of the TCV and/or its cargo. In some embodiments, the manufacturing method of a TCV according to the present disclosure may contribute to such a characteristic.

In some embodiments, TCVs may be stored at a freezing temperature. In some embodiments, when a TCV is prepared, a cryoprotectant may be added. In some embodiments, a cryoprotectant may comprise a sugar-based molecule. Non-limiting examples of cryoprotectants include sucrose, trehalose, and a combination thereof.

In some embodiments, the TCV may further comprise at least one cryoprotectant. In certain embodiments, the at least one cryoprotectant may be or may comprise a sugar-based molecule, e.g., a sugar molecule or a derivative thereof. In particular embodiments, the at least one cryoprotectant may be sucrose, trehalose, or a combination thereof. In a particular embodiment, the at least one cryoprotectant may be sucrose.

In some embodiments, a pharmaceutical composition according to the present disclosure may comprise at least one cryoprotectant. In certain embodiments, the cryoprotectant in a pharmaceutical composition may be the cryoprotectant comprised in the TCV contained in the pharmaceutical composition.

In some embodiments, the TCV, which may comprise at least one cryoprotectant, may be stable at a freezing temperature, optionally at about −20° C. or about −80° C., optionally for at least about one week, at least about two weeks, at least about three weeks, at least about a month, at least about two months, at least about four months, at least about five months, at least about 6 months, at least about 9 months, at least about a year, or at least about two year, or longer, or about one week to about two year, about two weeks to about a year, about three weeks to about nine month, about one to about six months, about one to five months, about one to four months, about one to three months, or about one to two months.

In some embodiments, the concentration of the at least one cryoprotectant contained in the TCV or pharmaceutical composition may be about 1% to about 40%, about 3% to about 30%, about 5% to about 30%, about 10% to about 20%, or about 15%.

A TCV according to the present disclosure may be prepared by any appropriate methods. In some embodiments, a TCV may be prepared by (a) generating a first solution by dissolving all components of the TCV in ethanol; (b) providing a second solution, which is aqueous; (c) combining the first and second solutions; and (d) removing ethanol, optionally by dialysis or evaporation.

In some embodiments, the first solution in step (a) may contain the TCV components at about 20-35 mM. In some embodiments, the second solution in step (b) may contain sodium acetate and/or sodium citrate, which optionally may be at about 25 mM. In some embodiments, the pH of the second solution in step (b) may be about 4 In some embodiments, the combining in step (c) may be by gentle mixing (optionally repeated manual reciprocation of the TCV-generating fluid in a pipette), micromixing optionally using a staggered herringbone micromixer (SHM), T-junction or Y-junction mixing, or extrusion. In a particular embodiment, the removing in step (d) is by dialysis.

### RNPs

In some embodiments, wherein the TCV comprise a RNP as a cargo, the RNP may be generated by any appropriate methods. In some embodiments, the RNP may be formed by mixing Cas9 and gRNA at an approximately equimolar ratio, optionally for about 5 minutes.

### Cargo Encapsulation by TCVs

In some embodiments wherein the TCV comprise a RNP as a cargo, the RNP encapsulation by TCVs may be performed by any appropriate methods. In some embodiments, the encapsulation may be performed by (i) providing an aqueous solution comprising the TCV; and (ii) mixing the at least one RNP with the aqueous solution, wherein mixing is effected under conditions suitable for the at least one RNP to be encapsulate within the TCV. In some embodiments, the aqueous solution in step (i) may have the pH of about 3 to about 8, optionally about 4 to about 7.5.

In some embodiments wherein the TCV comprise a nucleic acid molecule (not RNPs) as a cargo, the nucleic acid molecule encapsulation by TCVs may be performed by any appropriate methods. In some embodiments, the encapsulation may be performed by: (i) providing an aqueous solution comprising the TCV; and (ii) mixing the nucleic acid molecule with the aqueous solution, wherein mixing is effected under conditions suitable for the at least one nucleic acid molecule to be encapsulate within the TCV. In some embodiments, the aqueous solution in step (i) may have the pH of about 3 to about 8, optionally about 4 to about 7.5.

### In Vivo Methods

In one aspect, a pharmaceutical composition may be used for in vivo purposes. In some embodiments, the pharmaceutical composition may be for effecting gene editing of a target gene such as a SCD-associated gene in vivo. In some embodiments, the pharmaceutical composition may be for effecting gene expression alteration of a target gene such as a SCD-associated gene in vivo. In some embodiments, the pharmaceutical composition may be for preventing, ameliorating, or treating SCD.

In the strategies currently tested in the clinic for providing gene editing or gene expression alteration on a SCD-associated gene for the purpose of treating SCD, gene editing or gene expression alteration occurs in autologous HSCs and/or HSPCs ex vivo. This means that the strategy requires the steps of harvesting of HSCs and/or HSPCs from a patient, ex vivo modification of the harvested HSCs and/or HSPCs, myeloablation, and administering the modified HSCs and/or HSPCs back to the patient, which requires lengthy steps that require sophisticated facilities and high cost.

In one aspect, one characteristic of the inventive method is that the method addresses such disadvantages and difficulties. In some embodiments, the method involves injection of a pharmaceutical composition according to the present disclosure directly into the bone marrow of a subject, e.g. a subject with SCD, SCA, HbSC, or HbS β-thalassaemia, or other subjects as disclosed in the following section.

In some embodiments, the method involves injection of a pharmaceutical composition according to the present disclosure into the peripheral circulation of a subject, e.g. a subject with SCD, SCA, HbSC, or HbS β-thalassaemia or other subjects as disclosed in the following section, in which stem cell mobilization is induced. Such methods avoid any of the complicated steps as explained above (steps from stem cell harvest to administering modified stem cells).

### Subject/Patient Population

In one aspect, an in vivo method according to the present disclosure may be applied to any subject who is in need of a pharmaceutical composition according to the present disclosure.

In some embodiments, a subject may have or have a risk of developing a disease involving a cell or bone marrow origin or a cell in the bone marrow. In some embodiments, a subject may have or have a risk of developing SCD. In such an embodiment, the subject may have at least one βS allele, i.e., the subject is in the fate of having SCD from birth.

In some embodiment, the subject may be at any age or at any stage of SCD. In certain embodiments, the subject is in the immediate post-natal period, optionally about 6 weeks old or younger. In certain embodiments, the subject is about 3 month old or younger. In certain embodiments, the subject still comprises sufficient amount of HbF relative to HbA. In some embodiments, the subject is has the HbF:HbA ratio of about 2:1, about 1:1, about 1:2, about 1:3, about 1:4, about 1:5, or about 1:10. In certain embodiments, the subject has not fully developed SCD and is prior to manifesting a symptom or complication.

Injection into the Bone Marrow

In some embodiments, the method involves injection of a pharmaceutical composition according to the present disclosure directly into the bone marrow of a patient.

### Intramarrow Injection

Injection into the bone marrow, intramarrow injection or Intraosseous infusion (10), may be performed via any appropriate methods. The intramarrow route is currently used in the clinic, e.g., for bone marrow transplant or for acute infusion when peripheral venous access is not available. Also, application for the treatment of hematopoietic malignancies is being proposed (Islam, Clin Case Rep. 2015 December; 3(12): 1026-1029.; Islam. Biomed J. Sci & Tech Res. 23(5)-2019.)

In some embodiments, intramarrow injection may be performed using any appropriate methods, e.g., by a slow bolus push, e.g., using a standard IO device such as but not limited to First Access for Shock and Trauma (FAST1), the EZ-IO, or the Bone Injection Gun (BIG) or a manual device such as the Jamshidi needle or the Diekman modified needle (Dornhofer and Kellar. Intraosseous Vascular Access. [Updated 2021 Jul. 26]. In: StatPearls [Internet] Treasure Island (FL); StatPearls Publishing; 2021 January-.). Other examples of devices and details on the injection methods may be found in Islam, J Clin Pathol. 2020 September; 73(0):552-556, or U.S. Pat. No. 10,265,481B2. In particular embodiments, an intramarrow needle comprising the length of about 50 to about 100 mm or about 70 to about 80 nm may be used.

In some embodiments, when the target cells are cells that have a potential to eventually differentiate into red blood cells, the direct injection may be performed into tibia, femur, sternum, skull, ribs, pelvis (e.g., iliac), or any combinations thereof, which are the bones in which erythropoiesis mainly takes place. As erythropoiesis in the tibia and femur declines by about age 25, in some embodiments, these bones may be a suitable injection site for patients up to younger patients up to about age 20 or about 25.

### Dosing Regimen

The pharmaceutical composition for intramarrow injection may comprise any amounts of cargo sufficient for effecting sufficient gene editing and/or gene expression alteration. In some embodiments, the pharmaceutical composition may comprise per mL about 300 pmol to about 30000 pmol, optionally about 500 to about 10000 pmol, about 1000 to about 5000 pmol, about 2000 to about 4000 pmol, about 2500 to about 3000 pmol, or about 2700 pmol of the RNP or the nucleic acid molecule. In particular embodiments, the pharmaceutical composition may comprise about 2700 pmol of the RNP or the nucleic acid molecule per mL.

The intramarrow injection may be given at any appropriate volume and/or speed suited for effecting sufficient gene editing and/or gene expression alteration. In some embodiments, injection of the pharmaceutical composition may be via a continuous flow of about 25 mL to 125 mL per minute, optionally about 25 mL to 50 mL per minute, about 50 mL to 100 mL per minute, about 100 mL to 125 mL per minute, about 40 mL to about 80 mL per minute, or about 50 mL to about 70 mL per minute.

In some embodiments, the TCVs encapsulating at least one cargo may be comprised in or loaded in a matrix or material that may be injected or implanted in the bone marrow. Any appropriate matrices or materials may be used. In some embodiments, use of such a matrix or material may lead to improved safety, for example by allowing a smaller thus safer dose to provide efficacy and/or lead to improved feasibility, for example by allowing gradual release of the TCVs, thereby offering less frequent need of injections or shorter injection duration. Non-limiting examples of such a matrix or material may be those described in Ho et al., Sci Adv. 2021 May 19; 7(21):eabg3217, Lee et al., Proc Natl Acad Sci USA. 2012 Nov. 27; 109(48):19638-43. Epub 2012 Nov. 12., and Shah et al., Nat Biotechnol. 2019 March; 37(3):293-302.

Injection into the Peripheral Circulation

In some embodiments, the method involves injection of a pharmaceutical composition according to the present disclosure into the peripheral circulation of a patient in which stem cell mobilization is induced.

### Stem Cell Mobilization

Stem cell mobilization may be induced in a subject via any appropriate methods. In some embodiments, at least one agent that promotes stem cell mobilization (from the bone marrow to the peripheral circulation) may be administered to the subject. In some embodiments, such at least one agent may be administered prior to or at the same time as injection of a pharmaceutical composition comprising a TCV encapsulating a cargo according to the present disclosure.

In some embodiments, exemplary agents for stem cell mobilization include but are not limited to G-CSF (filgrastim), GM-CSF, Plerixafor, SCF, CXCR4 antagonists (e.g., POL6326, BKT-140, TG-0054), CXCL12 neutralizers (e.g., NOX-A12), Sphingosine-1-phosphate (SIP) antagonists (e.g., SEW2871), VCAM/VLA-4 inhibitors (e.g., BIO 5192), parathyroid hormone, protease inhibitors (e.g., Bortezomib), Grop (e.g., SB-251353), and hypoxia inducible factor (HIF) stabilizers (e.g., FG-4497). In particular embodiments, the stem cell mobilization agent may be G-CSF (filgrastim). In particular embodiments, the stem cell mobilization agent may be Plerixafor. In particular embodiments, the stem cell mobilization agent may be G-CSF (filgrastim) and Plerixafor.

The at least one stem cell mobilization agents may be administered in any appropriate manner (dose, route, frequency) that would provide sufficient and timely mobilization. Exemplary dosing may be described in the art, for example in Andreola et al., Eur J Haematol. 2012 February; 88(2):154-8. Epub 2011 Nov. 17. In some embodiments, stem cell mobilization may be induced by intravenous administration of G-CSF and plerixafor prior to injection of a pharmaceutical composition comprising a TCV encapsulating a cargo according to the present disclosure. In some embodiments, stem cell mobilization may be induced by intravenous administration of G-CSF followed by intravenous administration of plerixafor prior to injection of a pharmaceutical composition comprising a TCV encapsulating a cargo according to the present disclosure.

In particular embodiments, the dosing of G-CSF may be about 5-30 μg/kg/day, preferably about 10 μg/kg/day, for about 3-5 days, preferably 4 days. In particular embodiments, the dosing of plerixafor may start once the peripheral blood CD34⁺ cells are <20 cells/μL and/or on the day of the last G-CSF administration (e.g., the 4^{th} day) or the following day. In particular embodiments, the dosing of plerixafor may be about 0.1-0.5 mg/kg, preferably about 0.2-0.3 mg/kg or about 0.24 mg/kg.

### Dosing Regimen

The pharmaceutical composition for injection into the peripheral circulation (e.g., intravenous (IV) injection) may comprise any amounts of cargo sufficient for effecting sufficient gene editing and/or gene expression alteration. In some embodiments, the pharmaceutical composition may comprise per mL about 300 pmol to about 30000 pmol, optionally about 500 to about 10000 pmol, about 1000 to about 5000 pmol, about 2000 to about 4000 pmol, about 2500 to about 3000 pmol, or about 2700 pmol of the RNP or the nucleic acid molecule. In particular embodiments, the pharmaceutical composition may comprise about 2700 pmol of the RNP or the nucleic acid molecule per mL.

The injection into the peripheral circulation may be given at any appropriate volume and/or speed suited for effecting sufficient gene editing and/or gene expression alteration. In some embodiments, injection of the pharmaceutical composition may be via a continuous flow of about 25 mL to 125 mL per minute, optionally about 25 mL to 50 mL per minute, about 50 mL to 100 mL per minute, about 100 mL to 125 mL per minute, about 40 mL to about 80 mL per minute, or about 50 mL to about 70 mL per minute.

The maximum number of circulating CD34+ cells (or HSCs and/or HPCSs) may be achieved about 5 days after last plerixafor administration, at which point the median number of CD34+ cells (or HSCs and/or HPCSs) may be about 60 per μL (Andreola et al., Eur J Haematol. 2012 February; 88(2): 154-8. Epub 2011 Nov. 17.). Therefore, in some embodiments, injection of a pharmaceutical composition comprising a TCV encapsulating a cargo according to the present disclosure may start once the peripheral blood CD34+ cells (or HSCs and/or HPCSs) are 60 cells/μL or more.

In some embodiments, a single injection or a first injection (if injection is to be repeated or continuous injection is intended) may take place about 3-7 days, about every 3-7 days, about 4-6 days, about every 4-6 days, about 5 days, or about every 5 days after the last plerixafor administration. In alternative embodiments, a series of injections may be given once daily, e.g., for one week following the last plerixafor administration.

In some embodiments, there may be a prolonged time period between administration of the composition comprising the cargo comprising TCVs into the peripheral circulation or bone marrow, e.g., if it is determined after treatment (see “Monitoring and dosing regimen adjustment” section infra), that the treated subject does not comprise a sufficient number of normal (gene-edited) cells in their peripheral circulation, e.g., HSCs, HSPCs, MPPs, CMPs, MEPs, HPCs, erythroid progenitors (e.g., BFU-E, CFU-E), proerythroblasts, erythroblasts (basophilic erythroblasts, early erythroblasts (e.g., type I, type II), polychromatic erythroblasts, intermediate erythroblasts, acidophilic erythroblasts, late erythroblasts, normoblasts, reticulocytes before nucleus expulsion, reticulocytes, or erythrocytes, or any combination thereof.

### Method of Preventing, Ameliorating, or Treating

In one aspect, a pharmaceutical composition comprising a TCV encapsulating a cargo according to the present disclosure may be for preventing, ameliorating, or treating a target disease. In some embodiments, the target disease may be a disease associated with cells of bone marrow origin and/or cells in the bone marrow. In some embodiments, the target disease may be SCD. In particular embodiments, the target disease may be SCA, HbSC, or HbS β-thalassaemia.

Such a preventative, amelioration, or therapeutic method may comprise any of the methods for effecting gene editing and/or gene expression alteration in one or more target cells in vivo disclosed herein.

In some embodiments, the preventative, amelioration, or therapeutic method may comprise further administering another agent, together with or separately from the pharmaceutical composition according to the present disclosure. In some embodiments, the other agent may be one or more erythropoiesis stimulating agents. In some embodiments, the one or more erythropoiesis stimulating agents may be any of such agents disclosed herein. In some embodiments, the other agent may be another agent for SCD. In some embodiments, such another agent for SCD may be hydroxyurea, L-glutamine oral powder, crizanlizumab, a general pain medication, voxelotor, or any combination thereof. In some embodiments, a synergistic effect may be achieved by combining a pharmaceutical composition according to the present disclosure and at least one other agent for treating SCD.

### Monitoring and Dosing Regimen Adjustment

The effect of any of the in vivo method may be monitored, and monitoring may be on any appropriate parameters. Non-limiting examples of such parameters include but are not limited to: (i) % HSCs and HSPCs in the blood or bone marrow with successful gene editing and/or gene expression alteration; (ii) the number of HSCs and HSPCs in the blood or bone marrow with successful gene editing and/or gene expression alteration; (iii) % HSCs and HSPCs expressing HbF in the blood or bone marrow (e.g., the target gene is BCL11A or KLF1); (iv) the number of HSCs and HSPCs expressing HbF in the blood or bone marrow (e.g., the target gene is BCL11A or KLF1); (v) the expression level of the at least one SCD-associated gene or gene product or molecule, optionally beta-globin, beta-globin (HbS variant), gamma-globin, HbF, HbA, BCL11A, and/or KLF1; and (vi) changes in the symptom, which optionally may be changes in the frequency and/or levels of pain, swelling of hands and feet, infection, growth, and/or symptoms associated with vision.

Monitoring may be effected periodically, e.g., weekly, every 2 weeks, monthly or every 2 months, to assess whether the subject comprises a sufficient number of normal (gene-edited) cells in their peripheral circulation, e.g., HSCs, HSPCs, MPPs, CMPs, MEPs, HPCs, erythroid progenitors (e.g., BFU-E, CFU-E), proerythroblasts, erythroblasts (basophilic erythroblasts, early erythroblasts (e.g., type I, type II), polychromatic erythroblasts, intermediate erythroblasts, acidophilic erythroblasts, late erythroblasts, normoblasts, reticulocytes before nucleus expulsion, reticulocytes, or erythrocytes, or any combination thereof. A sufficient amount refers to the number of gene-edited which is determined to preclude or inhibit the symptoms of SCD, SCD, SCA, HbSC, or HbS β-thalassaemia, such as sickle cell crisis, vaso-occlusive crisis, acute cell syndrome, aplastic crisis, hemolytic crisis and the like. This will typically involve collecting blood samples from the subject periodically and assaying the genome of the collected peripheral cells thereof in order to determine the approximate number of gene-edited cells therein.

In any of the in vivo methods disclosed herein, the dosing regimen (such as dose, frequency, injection duration, etc) may be adjusted based on such monitoring.

Regardless of the injection route, the injection may be repeated as many times as need to for providing sufficient gene editing and/or gene expression alteration and/or sufficient prevention, amelioration, or treatment outcome.

In some embodiments, the injection may be given two or more times, to reach e.g., a minimum of about 10%, about 15%, about 20%, about 30%, or an about final 15-30% or about final 20-40% HSCs and HSPCs with successful gene editing and/or gene expression alteration among the total HSCs and HSPCs (in the bone marrow or in the peripheral circulation). In some embodiments, the injection may be given two or more times, to reach e.g., a minimum of about 10%, about 15%, about 20%, about 30%, or an about final 15-30% or about final 20-40% HSCs and HSPCs with wildtype beta-globin expression (e.g., when the target gene is the HbS variant of HBB) among the total HSCs and HSPCs (in the bone marrow or in the peripheral circulation). In some embodiments, the injection may be given two or more times, to reach e.g., a minimum of about 10%, about 15%, about 20%, about 30%, or an about final 15-30% or about final 20-40% HSCs and HSPCs with HbF expression (e.g., when the target gene is BCL11A or KLF1) among the total HSCs and HSPCs (in the bone marrow or in the peripheral circulation).

Regardless of the injection route, the injection may be repeated at any appropriate frequency for providing sufficient gene editing and/or gene expression alteration. In some embodiments, the injection may be given two or more times, optionally about 3-5 time, optionally about once a week, about every 2 weeks, or about every 3 weeks, about once a month, about every 3 months, about every 6 months, or about once per year.

In some embodiments, the level of successfully modified (by gene editing or gene expression alteration) target cells (or differentiated cells thereof) may be monitored. In some embodiments, the level of cells expressing the intended phenotype (e.g., expression of wildtype beta-globin or expression of HbF) may be monitored. In some embodiments, the dosing regimen such as injection dose, speed, or frequency may be adjusted based on the observation made during such monitoring.

### Definitions

All references cited herein, including patent documents and non-patent documents, are hereby incorporated by reference in their entirety. Nothing herein is to be construed as an admission that the present invention is not entitled to antedate such disclosure by virtue of prior invention.

Although various embodiments and examples of the present invention have been described referring to certain molecules, compositions, methods, or protocols, it is to be understood that the present invention is not limited to the particular molecules, compositions, methods, or protocols described herein, as theses may vary. It is also to be understood that the terminology used in the description is for the purpose of describing the particular versions or embodiments only and is not intended to limit the scope of the present invention which will be limited only by the appended claims.

It should be understood that, unless clearly indicated otherwise, in any methods disclosed or claimed herein that comprise more than one step, the order of the steps to be performed is not restricted by the order of the steps specifically cited.

Unless defined otherwise, all technical and scientific terms used herein have the same meaning as commonly understood by one of ordinary skill in the art to which this disclosure belongs.

It must also be noted that, unless the context clearly dictates otherwise, the singular forms “a,” “an,” and “the” as used herein and in the appended claims include plural refence. Thus, the reference to “a cell” refers to one or more cells and equivalents thereof known to those skilled in the art, and so forth. Unless defined otherwise, all technical and scientific terms used herein have the same meanings as commonly understood by a person of skilled in the art.

The term “about” or “approximately” means within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

In the specification above and in the appended claims, all transitional phrases such as “comprising,” “including,” “having,” “containing,” “involving,” “composed of,” and the like are to be understood to be open-ended, namely, to mean including but not limited to. Only the transitional phrases “consisting of” and “consisting essentially of” shall be closed or semi-closed transitional phrases, respectively.

“BAF chromatin remodeling complex subunit BCL11A”, also referred to herein as “BCL11A”, is known to function as a repressor of the HBG1 and HBG2 genes and thus a key regulator of the switch from HbF to HbA. BCL11A may have an amino acid sequence provided as GenBank: ADL14508.1. In one aspect, human BCL11A has the amino acid sequence provided as SEQ ID NO: 6 or the equivalent residues from a non-human species, e.g., mouse, rodent, monkey, ape and the like. In humans, BCL11A is encoded by the BCL11A gene on chromosome 2, with gene location 2p16.1 at nucleotide positions 60450520 to 60/553,654 (according to Gene Assembly GRCh38.p13), which encodes nine exons (NCBI, Gene ID: 53335). In one aspect, the BCL11A gene may have the polynucleotide sequence provided as NCBI Reference Sequence: NC_000002.12.

The term “cargo” or “cargo molecule” as used herein is one or more materials carried by and/or encapsulated by/in a TCV according to the present disclosure. In some embodiments, the combination of materials carried by a TCV may be collectively referred to as a “cargo”. For example, a TCV may carry a combination of a nuclease protein (such as Cas9) and a guide RNA (such as one comprising a sequence complementary to a target sequence) as a cargo. In some embodiments, a TCV may carry as a cargo a siRNA or a shRNA comprising a sequence complementary to a target sequence. In some embodiments, a TCV may carry as a cargo a miRNA comprising a sequence partially complementary (i.e., the percent complementarity is less than 100%) to a target sequence.

The term “cholesterol derivative” as used herein, in its broadest sense, encompasses any derivatives of cholesterol. Non-limiting examples of cholesterol derivatives include: DC-Chol (N,N-dimethyl-N-ethylcarboxamidocholesterol), 1,4-bis(3-N-oleylamino-propyl)piperazine (Gao, et al. Biochem, Biophys. Res. Comm. 179, 280 (1991); Wolf et al, Biotechniques 23, 139 (1997; U.S. Pat. No. 5,744,335), or imidazole cholesterol ester (ICE) (US20210220273A1).

Clustered regularly interspaced short palindromic repeats (CRISPR)/CRISPR-associated (Cas) systems are a class of genome-editing tools that target desired genomic sites in mammalian cells. A CRISPR/Cas system involves at least one Cas nuclease and a gRNA. Typically, the Cas nuclease may recognize a protospacer adjacent motif (PAM) sequence specific to the Cas nuclease in the target gene (sense or antisense strand) and if the gRNA is able to hybridize with a target sequence in the target gene proximate to the PAM site, the Cas nuclease may mediate cleavage of the target gene at about 2-6 nucleotides upstream of the PAM site. For example, the PAM sequence for Cas9 is 5′-NGG-3′. Type II CRISPR/Cas systems use Cas9 nuclease that is targeted to a genomic site by complexing with a guide RNA that hybridizes to an approximately 17-24-nucleotide DNA sequence immediately preceding an 5′-NGG-3′ motif (where “N” can be any nucleotide) recognized by Cas9 (thus, a (N)₂₀NGG target DNA sequence). This results in a double-strand break between the third and fourth nucleotides upstream of the NGG motif. The double strand break instigates either non-homologous end-joining (NHEJ), which typically leads to the introduction of one or more nucleotide insertions or deletions resulting in frameshift mutations that knock out gene alleles (e.g., nonsense-mediated mRNA decay (NMD)), or homology-directed repair (HDR), which can be exploited with the use of an exogenously introduced double-strand or single-strand DNA repair template to knock in or correct a mutation in the genome.

The term “destabilizing agent” as used herein encompasses any agents that destabilizes the cargo of a TCV according to the present disclosure. In some embodiments, a destabilizing agent may destabilize or degrade a nucleic acid cargo such as a gRNA, a protein cargo such as Cas nuclease, and/or a RNP. Exemplary destabilizing agents include but are not limited to: organic solvents such as ethanol and detergents such as sodium dodecyl sulfate. In some embodiments, a TCV may be substantially free of destabilizing agents. In some embodiments, such a TCV may be

The term “disease-associated gene” as used herein refers to a gene that is involved in and/or contributes to the pathogenesis or pathology of a disease or condition. Disease may be any disease such as but not limited to hematological diseases and cancer. In some embodiments, the disease is SCD.

The term “erythropoiesis” as used herein refers to the general process in which hematopoietic stem cells (HSCs) or hematopoietic stem and progenitor cells (HSCs) develop into mature erythrocytes (Zivot et al., Mol Med. 2018 Mar. 23; 24(1):11). HSCs or HSPCs, both of which have self-renewal capacity, give rise to multipotent progenitors (MPPs, also called short-term HSCs), which may develop directly into megakaryocyte-erythroid progenitors (MEPs) or first into common myeloid progenitors (CMPs) and then into MEPs (Erasmus et al., Cold Spring Harb Perspect Med 2013; 3:a011601). Cells in the stages from MPPs to MEPs belong to hematopoietic progenitor cells (HPCs), which do not have self-renewal capacity (Ferrari et al., Nat Rev Genet. 2021 April; 22(4):216-234.). Subsequently, MEPs may develop into erythroid progenitors of different differentiation levels (burst-forming unit erythroid cells (BFU-E); then colony-forming unit erythroid cells (CFU-E)), followed by proerythroblasts, and then erythroblasts of different differentiation levels (basophilic erythroblasts, also called early erythroblasts (further classified into type I and type II); polychromatic erythroblasts, also called intermediate erythroblasts; and acidophilic erythroblasts, also called late erythroblasts) (Valent et al., Haematologica, 2018 October; 103(10):1593-1603.). Healthy erythroblasts may also be referred to as normoblasts. Late erythroblasts then go through expulsion of the nucleus, which leads to the formation of reticulocytes. Reticulocytes exits the site of erythropoiesis (e.g., yolk sac, liver, spleen, or bone marrow) and enter the bloodstream. RNA and micro-organelles contained in reticulocytes mediate further protein synthesis, which promotes maturation of reticulocytes into erythrocytes, i.e., mature red blood cells (RBCs) (Lee et al., Blood Cells Mol Dis. June-August 2014; 53(1-2):1-10.).

Various pathways involving various factors (e.g., cytokines, cell surface receptors, transcription factors, etc) mediate erythropoiesis (Valent et al., Haematologica, 2018 October; 103(10):1593-1603; Sinclair, Biologics. 2013; 7:161-74. Epub 2013 Jul. 3.). For example, interleukin-3 (IL-3), granulocyte-macrophage colony-stimulating factor (GM-CSF), and granulocyte colony-stimulating factor (G-CSF) mediate development from CMPs and MEPs into BFU-E. IL-3, GM-CSF, IL-9, and insulin-like growth factor-1 (IGF-1) promote development during the BFU-E stage (early stage to late stage). IL-3, GM-CSF, IL-9, and erythropoietin (EPO) promote development from BFU-E to CFU-E, and EPO further promotes development of CFU-E into proerythroblasts. Effects mediated by EPO via EPO receptor are dependent on iron-metabolism and the interaction between the death receptor FAS and its ligand (FAS-L). Development during the erythroid progenitor up to proerythroblast stages are also regulated by broadly acting hematopoietic cytokines including stem cell factor (SCF) and the SCF receptor KIT. Transferrin (Tf) and Tf-receptor-1 (TfR-1) are additional major regulators of erythropoiesis, and growth differentiating factor 11 (GDF11) and polymeric immunoglobulin A (IgA) are considered to be involved in the regulation of certain stages of erythropoiesis. FAS-L and GDF11 seem to be particularly involved in the final maturation stage that leads to the generation of erythrocytes. As for transcription factors, GATA-1 triggers erythropoiesis by regulating the transcription of several erythroid differentiation-related genes, including genes involved in heme and/or globin synthesis, glycophorins, anti-apoptotic genes of the BH-3 family, genes involved in cell cycle regulation, and the gene for EPO receptor and therefor is the main regulator of lineage commitment, differentiation and survival of erythroid progenitors. Caspase is activated during the erythroblast stages and various caspase targets are affected, including Rock-1, Lamin B and Acinus. However, GATA-1 is protected from caspase cleavage by heat shock protein 70 (HSP70). Other factors (e.g., Activin A, TGF-beta, MCL-1, BCL-xL, HSP70, vitamin B12, folic acid, copper, ferritin, ferroportin, hepcidin) and transcription factors (e.g., STAT5A and STAT5B) and negative regulators (e.g., inhibin, TGF-beta, BID, caspases) also play a role in erythropoiesis regulation, which is reviewed in detail in, e.g., Valent et al., Haematologica. 2018 October; 103(10):1593-1603.

The location that erythropoiesis takes place in depends on the developmental stage of an organism. In humans, during the fetal stage, erythropoiesis occurs in the blood islands of the yolk sac in the first 8 weeks of gestation and then the fetal liver between 8 and 32 weeks of gestation (Philipsen, Haematologica. 2014 November; 99(11):1647-8.; Sankaran et al., Br J Haematol, 2010 April; 149(2):181-94. Epub 2010 Mar. 1.). The majority of erythropoiesis starts occurring in the bone marrow at around 32 weeks of gestation. Around birth the spleen serves as a transient erythropoietic organ, and the bone marrow takes over the majority erythropoiesis in about three months. The sites of erythropoiesis further change over time; red blood cell production recedes in the long bones (tibia, femur) by about age 25 and persists in the flat bones (sternum, skull, ribs, pelvis (e.g., iliac)), while minor contributions from the liver and less so from the spleen (Horn et al., Immunol Res. 2015 December; 63(1-3):75-89.). Once reticulocytes are formed, reticulocytes enter into the circulation. The site of erythropoiesis within the fetal liver, spleen (specifically the red pulp), and bone marrow is called erythroblastic islands (Wanwani and Bieker, Curr Top Dev Biol. 2008; 82: 23-53.).

“Guide RNA” or “gRNA”, as used herein in relation to the CRISPR/Cas gene editing, refers to a piece of a RNA fragment that binds to a target DNA sequence and guide a Cas nuclease protein to the specific site of gene editing. In CRISPR/Cas gene editing, a gRNA may comprise or consist of: a crispr RNA (crRNA), which comprises a target-complementary sequence of about 15-75 nucleotides that is complementary to the target DNA sequence; and a trans-activating crispr RNA (tracrRNA), which serves as a binding scaffold for the Cas nuclease. A gRNA may be comprise the two parts (crRNA and tracrRNA) linked forming a single molecule, or a gRNA may be a complex of a crRNA molecule and a trcrRNA molecule. When Cas9 is used, in some embodiments, the target-complementary sequence may comprise a GC content in the range of 40-80%, and in some embodiments, and the target-complementary sequence may have a length of 17-24 nucleotides. A gRNA may be a single-stranded gRNA (sgRNA) molecule.

“Guide RNA” or “gRNA”, as used herein in relation to the CRISPR/Cas gene editing (also referred to as “CRISPR-mediated gene editing), refers to a RNA fragment (e.g., single guide RNA (“sgRNA”)) or a hybrid of two RNA fragments (e.g., dual guide RNA (“dgRNA”)) that binds to a target DNA sequence and guide a Cas endonuclease protein to the specific site of a DNA (e.g., in a genome) to allow for Cas-mediated cleavage of a DNA molecule. In some embodiments, gRNA may be dgRNA comprising: (I) a crispr RNA (crRNA), which comprises (i) a target-complementary sequence of about 15-75 nucleotides that is complementary to (or comprising some mismatches relative to) the target DNA sequence and (ii) a crRNA flagpole sequence; and (II) a trans-activating crispr RNA (tracrRNA), which comprises (i) a tracrRNA flagpole sequence and (ii) tracrRNA endonuclease binding domain, which serves as a binding scaffold for the Cas endonuclease, wherein the crRNA and tracrRNA hybridize with each other via the flagpole sequences. In some embodiments, a gRNA may be sgRNA comprising (I) a crRNA sequence linked to (II) a trarRNA sequence as a single polynucleotide.

In some embodiments, the dgRNA and sgRNA may have the following formats:

dgRNA
crRNA (polynucleotide 1 having a crRNA sequence):
[target-complementary sequence]-[crRNA flagpole sequence]-[(optional) crRNA first flagpole extension]-[(optional) crRNA second flagpole extension]
* the sequence of [crRNA flagpole sequence]-[(optional) crRNA first flagpole extension]-[(optional) crRNA second flagpole extension] may be referred to herein as “crRNA backbone sequence”.
tracrRNA (polynucleotide 2 having a tracrRNA):
[(optional) tracrRNA first extension]-[tracrRNA flagpole sequence]-[tracrRNA endonuclease binding domain]
   sgRNA (Having a crRNA Sequence Linked to a tracrRNA Sequence)
[target-complementary sequence]-[crRNA flagpole sequence]-[(optional) crRNA first flagpole extension]-[(optional) linker]-[(optional) tracrRNA first extension]-[tracrRNA flagpole sequence]-[tracrRNA endonuclease binding domain]
* the sequence of [crRNA flagpole sequence]-[(optional) crRNA first flagpole extension]-[(optional) linker]-[(optional) tracrRNA first extension]-[tracrRNA flagpole sequence]-[tracrRNA endonuclease binding domain] may be referred to herein as “sgRNA backbone sequence”.

In some embodiments, the crRNA flagpole sequence may comprise SEQ ID NO: 131 or 132. In some embodiments, the optional crRNA first flagpole extension may comprise SEQ ID NO: 133. In some embodiments, the optional crRNA second flagpole extension may comprise SEQ ID NO: 134. In some embodiments, the optional tracrRNA first extension may comprise SEQ ID NO: 135. In some embodiments, the tracrRNA flagpole sequence may comprise SEQ ID NO: 136 or 137. In some embodiments, the tracrRNA endonuclease binding domain may comprise SEQ ID NO: 138. In some embodiments, the tracrRNA endonuclease binding domain may further comprise or may be followed by one or more uracil based, e.g., 5′-U-3′, 5′-UU-3′, 5′-UUU-3′, 5′-UUUU-3′, 5′-UUUUU-3′, 5′-UUUUUU-3′, 5′-UUUUUUU-3′, or 5′-UUUUUUUU-3′.

In certain embodiments, the crRNA flagpole sequence may comprise SEQ ID NO: 131 and the tracrRNA flagpole sequence may comprise SEQ ID NO: 136. In certain embodiments, the crRNA flagpole sequence may comprise SEQ ID NO: 132 and the tracrRNA flagpole sequence may comprise SEQ ID NO: 137. In some embodiments, the optional linker which links a crRNA and tracrRNA in a sgRNA may comprise or consist of SEQ ID NO: 139.

In some embodiments, a sgRNA may comprise a sgRNA backbone sequence (the sequence which is placed 3′ to a target-complementary sequence in a sgRNA) of any of SEQ ID NOS: 141-144. In certain embodiments, the sgRNA backbone sequence may be followed by one or more uracils. In particular embodiments, the sgRNA backbone sequence may be followed by 1-10 uracils, such as 3 uracils, 4 uracils, 5 uracils, 6 uracils, 7 uracils, or 8 uracils.

In some embodiments, a dgRNA may comprise (I) a crRNA sequence comprising a crRNA backbone sequence (the sequence which is placed 3′ to a target-complementary sequence in a crRNA) comprising SEQ ID NO: 145 and (II) a tracrRNA sequence comprising SEQ ID NO: 146. In some embodiments, a dgRNA may comprise (I) a crRNA sequence comprising a sgRNA backbone sequence (the sequence which is placed 3′ to a target-complementary sequence in a crRNA) comprising SEQ ID NO: 147 and (II) a tracrRNA sequence comprising SEQ ID NO: 148.

When Cas9 is used, in some embodiments, the target-complementary sequence may comprise a GC content in the range of 40-80%, and in some embodiments, and the target-complementary sequence may have a length of 17-24 nucleotides.

A target-complementary sequence of a gRNA may be any appropriate length. While the most frequently used target-complementary sequence length is 20 nt, a longer or shorter target-complementary sequence may also be used. In some embodiments, a gRNA longer than 20 nt may be used. For example, Ran et al. demonstrated that longer gRNAs are commonly cleaved to a shorter length so that the target-complementary sequence is e.g., 20 nt and thus the complementarity in the segment in excess of 20 nt may not be important, i.e., may or may not be complementary to a target sequence (Ran et al., Cell. 2013 Sep. 12; 154(6):1380-9.). In some embodiments, a gRNA shorter than 20 nt may be used. For example, Fu et al. demonstrated that truncated (i.e., <20 nt) gRNAs, which is as short as 17, 18, or 19 nt, may also target the same target as a corresponding 20 nt-long gRNA and perhaps even may have decreased off-target effects (Fu et al., Nat Biotechnol. 2014 March; 32(3): 279-28).

A target-complementary sequence of a gRNA may or may not comprise a mismatch relative to the target sequence. In some cases, a mismatch at a particular position may reduce gRNA specificity to the target sequence. For example, in the context of SpCas9, Cong et al demonstrated that complementarity at up to 11 nt from the 3′-end of a target-complementary sequence is more important than that at a more upstream region (Cong et al., Science. 2-13 February 15; 339(6121): 819-823.). Again in the context of SpCas9, Zheng et al demonstrated that the core sequence which is from the 4^{th} to the 7^{th} nt from the 3′-end is more sensitive to target mismatch compared to the rest of the target-complementary sequence g p. 23 Therefore, in some embodiments, a gRNA target-complementary sequence may comprise a mismatch relative to its target sequence outside of such a core position.

The term “helper lipid” or “structural lipid” as used herein refers to a type of lipid that may be comprised in a TCV in addition to an ionizable cationic lipid. In some embodiments, a helper lipid may be a non-cationic lipid and may be neutral, zwitterionic, or anionic lipid. In some embodiments, a helper lipid may be a lipid that carries a net negative charge at a selected pH, such as physiological pH. Without wishing to be bound by theory, helper lipids in TCVs in general are used to provide particle stability and/or biocompatibility and/or to enhance cargo delivery efficiency. Non-limiting helper lipids include, but are not limited to dioleoylphosphatidylethanolamine (DOPE), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), or a mixture thereof. In some embodiments, a helper lipid is dioleoylphosphatidylethanolamine (DOPE).

“Hemoglobin”, “haemoglobin”, “Hb”, or “Hgb” is a tetrameric protein composed of both alpha-like and beta-like globin subunits (Sankaran et al., Br J Haematol. 2010 April; 149(2):181-94. Epub 2010 Mar. 1.). Each globin subunit is associated with the cofactor hem (also called haem), which can carry an oxygen molecule. In humans, genes encoding alpha-like globin subunits are located in the alpha-globin locus on Chromosome 16 and are under the control of a set of distal enhancers referred to as the multispecies conserved sequences (MCS); and genes encoding beta-like globin subunits are located in the beta-globin locus on Chromosome 11 and are under the control of a set of distal enhancers referred to as the locus control region (LCR) (Barbarani et al., Front Cell Dev Biol. 2021 Apr. 1; 9:640060.). The alpha-globin locus contains three functional alpha-like globin genes: the embryonic HBZ gene, which encodes zeta-globin, and the two fetal/adult HBA2 and HBA1 duplicated genes, which encode alpha2-globin and alpha1-globin, respectively. The beta-globin locus contains five functional beta-like globin genes: the embryonic HBE gene, which encodes epsilon-globin, the two highly homologous fetal HBG2 and HBG1 genes, which encode G-gamma-globin (hemoglobin subunit gamma 2) and A-gamma-globin (hemoglobin subunit gamma 1) (which only differ by a single amino acid), respectively, and the two adult HBD and HBB genes, which encode delta-globin and beta-globin, respectively. Beta-globin accounts for about 98% of adult beta-like globin. The genes contained in the alpha- and beta-globin loci are sequentially expressed in a stage-specific manner that maintains the 1:1 ratio between the alpha-like and beta-like globin chains, in a process known as “hemoglobin switching”. Hb is mostly found in cells of the erythrocyte lineage. Hb synthesis starts at around the proerythroblast stage, and Hb continues to accumulate as proerythroblasts develop into basophilic, polychromatophilic, and orthochromatic erythroblasts (Zivot et al., Mol Med. 2018 Mar. 23; 24(1):11.).

“Hemoglobin subunit beta”, also referred to herein as “beta-globin”, “beta-globin subunit”, “β-globin”, “β-globin subunit”, “HBB”, or the like, is a component of the adult hemoglobin (HbA). Human beta-globin may have an amino acid sequence provided as NCBI Reference Sequence: NP_000509.1. In one aspect, human beta-globin has the amino acid sequence provided as SEQ ID NO: 1 or the equivalent residues from a non-human species, e.g., mouse, rodent, monkey, ape and the like. In humans, beta-globin is encoded by the HBB gene on chromosome 11, with gene location 11p15.4 (Gene Assembly GRCh38.p13) (NCBI, Gene ID: 3043). In one aspect, the wildtype HBB gene may have the polynucleotide sequence provided as SEQ ID NO: 11 (corresponding to nucleotide positions 5225464 to 5227071 of Chromosome 11 (according to Gene Assembly GRCh38.p13)), with the open reading frame sequence provided as SEQ ID NO: 12 (corresponding to positions 5225601 to 5227021 of Chromosome 11), encoding three exons. In some embodiments, human beta-globin may be encoded by a cDNA comprising the polynucleotide sequence of SEQ ID NO: 13.

Several diseases and/or phenotypes are caused by one or more alterations in the HBB gene. According to the OMIM® database (https://www.omm.org/), such diseases and/or phenotypes include: Delta-beta thalassemia (Phenotype MIM number 141749, autosomal dominant); Erythrocytosis 6 (Phenotype MIM number 617980, autosomal dominant); Heinz body anemia (Phenotype MIM number 140700, autosomal dominant); Hereditary persistence of fetal hemoglobin (Phenotype MIM number 141749, autosomal dominant); Methemoglobinemia, beta type (Phenotype MIM number 617971, autosomal dominant); Sickle cell anemia (Phenotype MIM number 603903, autosomal recessive); Thalassemia, beta (Phenotype MIM number 613985); Thalassemia-beta, dominant inclusion-body beta (Phenotype MIM number 603902, autosomal recessive; and resistance to Malaria, resistance to (Phenotype MIM number 611162).

“Hemoglobin subunit gamma”, also referred to herein as “gamma-globin”, “gamma-globin subunit”, “γ-globin”, “γ-globin subunit”, “HBG”, or the like, is a component of the fetal hemoglobin (HbF). Human hemoglobin subunit gamma 1 may have an amino acid sequence provided as GenBank: EAW68804.1. In one aspect, human hemoglobin subunit gamma 1 has the amino acid sequence provided as SEQ ID NO: 8 or the equivalent residues from a non-human species, e.g., mouse, rodent, monkey, ape and the like. In humans, hemoglobin subunit gamma 1 is encoded by the HBG1 gene on chromosome 11, with gene location 11p15.4 (Gene Assembly GRCh38.p14) (NCBI, Gene ID: 3047). In one aspect, the wildtype HBG1 gene may have the polynucleotide sequence corresponding to nucleotide positions 5248269 to 5249857 of Chromosome 11 (according to Gene Assembly GRCh38.p14)), encoding three exons. Human hemoglobin subunit gamma 2 may have an amino acid sequence provided as GenBank: AAI30460.1. In one aspect, human hemoglobin subunit gamma 2 has the amino acid sequence provided as SEQ ID NO: 9 or the equivalent residues from a non-human species, e.g., mouse, rodent, monkey, ape and the like. In humans, hemoglobin subunit gamma 2 is encoded by the HBG2 gene on chromosome 11, with gene location 11p15.4 (Gene Assembly GRCh38.p14) (NCBI, Gene ID: 3048). In one aspect, the wildtype HBG2 gene may have the polynucleotide sequence corresponding to nucleotide positions 5253188 to 5254781 of Chromosome 11 (according to Gene Assembly GRCh38.p14)), encoding three exons.

“Hemoglobin switch” or “hemoglobin switching” is the process of developmental stage-specific expression of different globin genes. In humans, for beta-like globins, around week 6 of gestation, embryonic globin (epsilon-globin) is silenced and fetal globin (gamma-globin, which is G-gamma-globin or A-gamma-globin) starts to be expressed. Perinatally, the switch to adult globin (beta-globin) occurs; and for the alpha-like globins, a single switch from the embryonic globin (zeta-globin) to the adult globin (alpha-globin, which is alpha2 or alpha1 globin) occurs (Philipsen, Haematologica. 2014 November; 99(11):1637-9.). Therefore, around birth, the most abundant Hb switches from the fetal hemoglobin (HbF), which is a tetramer of two alpha and two gamma globins (α2γ2), to the adult hemoglobin (HbA) ((>90%) form of adult Hb), which is a tetramer of two alpha and two beta globins (α2β2) (Kato et al., Nat Rev Dis Primers. 2018 Mar. 15; 4:18010.). This switch is normally completed during infancy and typically lasts until approximately 6 months of age (Sankaran et al., Br J Haematol. 2010 April; 149(2):181-94, Epub 2010 Mar. 1.). Notably, HbF binds oxygen with greater affinity than HbA, being functional when reactivated in adults (Lamsfus-Calle et al., Sci Rep 2020 Jun. 23; 10(1):10133.).

A variety of nuclear factors involved in transcriptional regulation have been suggested to be involved in globin gene regulation and switching. Such nuclear factors include but are not limited to: BCL11A, KLF1, SOX6, GATA1, NF-E4, COUP-TF, DRED/TR2/TR4, MBD2, Ikaros-PYR complex, and BRG1 (the catalytic subunit of the SWI/SNF complex) (Sankaran et al., Br J Haematol. 2010 April; 149(2):181-94. Epub 2010 Mar. 1.). For example, BCL11A is a repressor of the HBG1 and HBG2 genes and thus a key regulator of the switch from HbF to HbA and is crucial for the maintenance of HbF silencing in humans; and KLF1 was discovered as an activator of the HBB gene. In fact, transduction of K562 cells with BCL11A or KLF1 increased the HBB transcript about 5.9- and 7.5-fold, respectively, and transduction of K562 cells with both BCL11A and KLF1 increased the HBB transcript about 300-890-fold (Trakarnsanga et al., Haematologica. 2014 November; 99(11):1677-85. Epub 2014 Aug. 8.). When erythroid cells differentiated from human iPS cell-derived erythroid progenitor-1 cells (HiDEP-1 cells, which express endogenous KLF1 at a level similar to adult erythroid cells) were transduced with BCL11A, a robust increase in the beta-globin protein expression was observed. As reviewed in Sankaran et al. (Sankaran et al., Br J Haematol. 2010 April; 149(2):181-94. Epub 2010 Mar. 1.): SOX6 seems to have a role in repressing HbF; GATA1 seems to repress HBG1 and/or HBG2 gene expression and have a direct role in Hb switching; NF-E4 increases HBG1 and HBG2 gene expression; COUP-TF is a repressor of HBG1 and HBG2 genes; DRED complex (heterodimer of nuclear orphan receptors TR2 and TR4) seems to repress expression of HBE1, HBG1 and HBG2 genes; MBD2 is a group of proteins (part of the methyl-CpG binding protein complex 1 (MeCP1), which contains the proteins Mi-2, MTA1, MTA2, MBD3, HDAC1, HDAC2, RbAp46 and RbAp48) and is a repressor of HBG1 and HBG2 genes; and Ikaros-PYR complex appears to promote Hb switching; and BRG1 (the catalytic subunit of the SWI/SNF complex) appears to active transcription of HBB.

The term “intra marrow”, “intraosseous”, “intraosseously”, “IO” as used herein the administration route involving direct injection into the bone marrow.

The term “ionizable cationic lipid” as used herein, refers to any lipid that carries a net neutral charge at about physiological pH but is capable of becoming positively charged at a lower pH, e.g., pH below about 7, below about 6.5, below about 6, below about 5.5, below about 5, or below about 4.5, typically below about 6, or between about 5 and 6.5, between about 5 and 6, or between about 5.5 and 6. Without wishing to be bound by theory, a net neutral charge helps toxicity, and positive charges under a low pH may be useful in forming a complex with a negatively charged cargo such as a nucleic acid molecule and/or protein. Becoming positive charges under as the pH decreases may also help release of the cargo from an endosome once in a cell (endosomal escape), e.g., by taking protons in an endosome thereby destabilizing and bursting the endosome. Examples of ionizable cationic lipids may include, for example, N,N-dimethyl-2,3-dioleyloxy)propylamine (DODMA), N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxyl)propyl)-N,N,N-trimethylammonium chloride (DOTMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA·Cl), 1,2-Dilinoleoyl-3-trimethylaminopropane chloride salt (DLin-TAR·Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLin-K-DMA), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyl)tetrahydro-3 aH-cyclopenta[d][1,3]dioxol-5-amine (ALNY-100), N,N-dimethyl-2,2-di-(9Z,12Z)-9,12-octadecadien-1-yl-1,3-dioxolane-4-ethanamine (KC2), (6Z,9Z,28Z,31Z)-heptatriaconta-6,9,28,31-tetraen-19-yl 4-(dimethylamino)butanoate (MC3), or a mixture thereof.

Additional examples of ionizable cationic lipids include, but are not limited to, N-(2,3-dioleyloxyl)propyl-N,N—N-triethylammonium chloride (“DOTMA”); 1,2-Dioleyloxy-3-trimethylaminopropane chloride salt (“DOTAP·Cl”); 3.beta.-(N—(N′,N′-dimethylaminoethane)-carbamoyl)cholesterol (“DC-Chol”), N-(1-(2,3-dioleyloxyl)propyl)-N-2-(sperminecarboxamido)ethyl)-N,N-dimethyl-ammonium trifluoracetate (“DOSPA”), dioctadecylamidoglycyl carboxyspermine (“DOGS”), 1,2-dioleoyl-3-dimethylammonium propane (“DODAP”), and N-(1,2-dimyristyloxyprop-3-yl)-N,N-dimethyl-N-hydroxyethyl ammonium bromide (“DMRIE”), and mixtures thereof. Additionally, a number of commercial preparations of cationic lipids can be used, such as, e.g., LIPOFECTIN (available from GIBCO/BRL), and LIPOFECTAMINE (available from GIBCO/BRL).

The term “complementary” or “complementarity” means that a nucleic acid can form hydrogen bond(s) with another nucleic acid sequence by either traditional Watson-Crick or other non-traditional types of interactions such as Wobble-base pairing which permits binding of guanine and uracil. A percent complementarity indicates the percentage of residues in a nucleic acid molecule that can form hydrogen bonds with a second nucleic acid sequence.

“Kruppel like factor 1”, also referred to herein as “KLF1”, is known to function as an activator of the HBB gene and thus also a key regulator of the switch from HbF to HbA. KLF1 may have an amino acid sequence provided as GenBank: AHA61454.1. In one aspect, human KLF1 has the amino acid sequence provided as SEQ ID NO: 7 or the equivalent residues from a non-human species, e.g., mouse, rodent, monkey, ape and the like. In humans, KLF1 is encoded by the KLF1 gene on chromosome 19, with gene location 19p13.13 at nucleotide positions 12884422 to 12887201 (according to Gene Assembly GRCh38.p13), which encodes three exons (NCBI, Gene ID: 10661). In one aspect, the KLF1 gene may have the polynucleotide sequence provided as NCBI Reference Sequence: NC_000019.10.

The term “mutation” or “point mutation” as used herein in relation to nucleic acid or nucleotide sequence means a change in a nucleotide in a DNA or RNA molecule. A mutation may be a change from a nucleotide to another nucleotide or deletion of a nucleotide or an insertion of a nucleotide. When a mutation causes replacement of a nucleotide with another nucleotide in an open reading frame, the mutation may cause an amino acid substitution (“missense mutation”) or appearance of an early stop codon (“nonsense mutation”) leading to a shorter protein product or may not cause any changes in the protein product (“silent mutation”). When a mutation causes insertion or deletion of a nucleotide in an open reading frame, unless the number of insertion or deletion is divisible by three, the mutation changes the grouping of the codons to be read (“frame shift mutation”), causing dramatic changes in the protein sequence.

“Lipid-based TCVs” as used in are TCVs that comprise at least one lipid and encompass lipid nanoparticles. In some embodiments, a lipid-based TCV may comprise at least one ionizable cationic lipid. In some embodiments, a lipid-based TCV may comprise at least one helper lipid. In some embodiments, a lipid-based TCV may comprise at least one phospholipid. In some embodiments, a lipid-based TCV may comprise at least one cholesterol (or cholesterol derivative). In some embodiments, a lipid-based TCV may comprise, essentially consist of, or consist of at least one ionizable cationic lipid, at least one helper lipid, at least one phospholipid, and at least one cholesterol (or cholesterol derivative), and optionally polyethyleneglycol (PEG) or PEG-lipid. Exemplary TCVs include but not are limited to those described in Applicant's WO2020077007A1. In some embodiments, a lipid-based TCV may comprise, essentially consist of, or consist of an ionizable cationic lipid, one or more phospholipids, and cholesterol, the ratio of which are about 20:30:10:40 in mol %. In some embodiments, a lipid-based TCV may comprise, essentially consist of, or consist of an ionizable cationic lipid, one or more phospholipids, cholesterol, and PEG-lipid, the ratio of which are about 20:30:10:39:1 in mol %. TCVs may be generated using gentle mixing such as repeated manual reciprocation of the TCV-generating fluid in a pipette, micromixing optionally using staggered herringbone micromixer (SHM) or T-junction or Y-junction mixing, or extrusion methods, or other TCV-mixing methods as desired.

The term “nuclease” as used herein refers to an enzyme capable of catalyzing the cleavage of phosphodiester bonds between nucleotides of nucleic acids. In the CRISPR/Cas system, which involves a gRNA and a CRISPR-associated (Cas) nuclease, the Cas nuclease recognizes a PAM sequence in the target gene (sense or antisense) and if the gRNA is able to hybridize with a target sequence of the target gene proximate to the PAM sequence, the Cas nuclease may mediate cleavage of the target gene at about 2-6 nucleotides upstream of the PAM. The PAM sequence is specific to the Cas nuclease. Any appropriate Cas nucleases may be used in the invention disclosed herein. Appropriate Cas nucleases include but are not limited to Cas9 of different bacterial species such as Streptococcus pyogenes (SpCas9, which recognizes the PAM sequence of 5′-NGG-3′), Staphylococcus aureus Cas9 (SaCas9, which recognizes the PAM sequence of 5′-NNGRRT-3′), Streptococcus thermophilus (StCas9, which recognizes the PAM sequence of 5′-NGGNG-3′), Neisseria meningitidis (NmCas9, which recognizes the PAM sequence of 5′-NNNNGATT-3′), Francisella novicida (FnCas9, which recognizes the PAM sequence of 5′-NG-3′), Campylobacter jejuni (CjCas9, which recognizes the PAM sequence of 5′-NNNNACA-3′), Streptococcus canis (ScCas9, which recognizes the PAM sequence of 5′-NNGG-3′), Staphylococcus auricularis (SauriCas9, which recognizes the PAM sequence of 5′-NNG-3′), or any engineered variants thereof, including but not limited to SaCas9-HF, SpCas9-HF1, KKHSaCas9, eSpCas9, HypaCas9, FokI-Fused dCas9, xCas9, SpRY (variant of SpCas9), SpG (variant of SpCas9), which are collectively referred to as Cas9 herein. Other Cas nuclease examples include Cas3, Cas8a2, Cas8b, Cas8c, Cas10, Csx11, Cas12, Cas12a or Cpf1, Cas13, Cas13a, C2c1, C2c3, and C2c2.

The terms “nucleic acid”, “nucleic acid molecule”, and “polynucleotide” are used interchangeably herein and encompass any compounds that comprise a polymer of nucleotides linked via a phosphodiester bond. Exemplary nucleic acids include but are not limited to RNA and DNA molecules, including molecules comprising cDNA, genomic DNA, synthetic DNA, and DNA or RNA molecules containing nucleic acid analogs. Nucleic acid molecules can have any three-dimensional structure. A nucleic acid molecule can be double-stranded or single-stranded (e.g., a sense strand or an antisense strand). Other non-limiting examples of nucleic acid molecules include genes, gene fragments, exons, introns, messenger RNA (mRNA), transfer RNA, ribosomal RNA, siRNA, microRNA, tracrRNAs, crRNAs, guide RNAs, ribozymes, cDNA, recombinant polynucleotides, branched polynucleotides, nucleic acid probes and nucleic acid primers. A nucleic acid molecule may contain unconventional or modified nucleotides. The terms “polynucleotide sequence” and “nucleic acid sequence” as used herein interchangeably refer to the sequence of a polynucleotide molecule. The nomenclature for nucleotide bases as set forth in 37 CFR § 1.822 is used herein.

The term “phospholipid” as used herein refers to any lipid comprising a phosphate group. Non-limiting examples of suitable phospholipids include: distearoylphosphatidylcholine (DSPC), dioleoyl phosphatidylethanolamine (DOPE), dipalmitoylphosphatidylcholine (DPPC), phosphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dilinoleoylphosphatidylcholine distearoylphophatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine, and combinations thereof. In one embodiment, the phospholipid is distearoylphosphatidylcholine (DSPC).

The term “polyethyleneglycol-lipid” or “PEG-lipid” as used herein refers to any lipid modified or conjugated to one or more polyethyleneglycol (PEG) molecules. Without wishing to be bound by theory, containing PEG or a PEG-lipid in a TCV may help maintain TCV particle size (keep a TCV from getting too big) and/or help maintain particle stability in vivo. Some examples of PEG-lipids that are useful in the present invention may have a variety of “anchoring” lipid portions to secure the PEG to the surface of the lipid-based TCVs. Non-limiting examples of suitable PEG-lipids include PEG-myristoyl diglyceride (PEG-DMG) (e.g., 1,2-dimyristoyl-rac-glycero-3-methoxypolyethylene glycol-2000 (Avanti® Polar Lipids (Birmingham, AL)), which is a mixture of 1,2-DMG PEG2000 and 1,3-DMG PEG2000 (e.g., in about 97:3 ratio)), PEG-phosphatidylethanolamine and phosphatidic acid, PEG-ceramide conjugates (e.g., PEG-CerC14 or PEG-CerC20) which are described in U.S. Pat. No. 5,820,873, incorporated herein by reference, PEG-modified dialkylamines, and PEG-modified 1,2-diacyloxypropan-3-amines. Particularly examples include PEG-modified diacylglycerols and dialkylglycerols.

The phrase “pharmaceutically acceptable” refers to molecular entities and compositions that are physiologically tolerable and do not typically produce an unintended and intolerable response such as an allergic response, when administered to a human. In some embodiments, the term “pharmaceutically acceptable”, as used herein, means approved by a regulatory agency of the Federal or a state government or listed in the U.S. Pharmacopeia or other generally recognized pharmacopeia for use in animals, and more particularly in humans.

The term “ribonucleoprotein”, “RNP”, or “RNP complex” as used herein refers to a complex of one or more RNA molecules and an RNA-binding protein. In the context of the CRISPR/Cas system, an RNP may be a complex of a gRNA and a Cas nuclease. The gRNA may be a RNA fragment containing a crRNA portion and a tracrRNA portion linked to each other or be a complex formed between a crRNA molecule and a tracrRNA molecule.

“Sickle cell disease” or “SCD”, as used herein, refers to a group of disorders caused by a mutation(s) in and/or altered expression of HBB, the gene encoding beta-globin (i.e., hemoglobin subunit beta) and may also be referred to as “β-hemoglobinopathies” (Kato et al., Nat Rev Dis Primers. 2018 Mar. 15; 4:18010.). Exemplary SCDs include but are not limited to sickle cell anemia (SCA), Sickle cell-hemoglobin C (HbSC), and HbS β-thalassaemia. HbA is a tetramer formed by two alpha-globin subunits and two beta-globin subunits, the latter of which are encoded by HBB. The sickle Hb (HbS) allele, βS, is an HBB allele in which an adenine-to-thymine (A-to-T) substitution (HBB wild type (SEQ ID NO: 11) to HBB βS allele (SEQ ID NO: 21)) results in the replacement of glutamic acid with valine at position 7 (Glu7Val) (HBB wild type (SEQ ID NO: 1) to HBB HbS variant (SEQ ID NO: 2)) in mature beta-globin (when the first methionine is counted as position 1). SCD occurs when both HBB alleles are mutated and at least one of them is the βS allele. Hemoglobin S (also referred to as sickle Hb or HbS) (containing beta-globin subunits encoded by the βS allele) that is deoxygenated (not bound to oxygen) can polymerize, and HbS polymers can stiffen the erythrocyte. Individuals with one βS allele have the sickle cell trait (HbAS) but not SCD; individuals with SCA, the most common SCD genotype, have two βS alleles (βS/βS).

Other relatively common SCD genotypes are also possible. Individuals with the HbSC genotype have one βS allele and one HBB allele with a different nucleotide substitution, βC allele, that generates another structural variant of Hb, hemoglobin C variant (HbC). βC is an HBB allele in which nucleic acid substitution (e.g., G to A; HBB wild type (SEQ ID NO: 11) to HBB βC allele (SEQ ID NO: 31)) results in the replacement of glutamic acid with lysine at position 7 (Glu7Lys) (HBB wild type (SEQ ID NO: 1) to HBB HbC variant (SEQ ID NO: 3)) in mature beta-globin (when the first methionine is counted as position 1). The βC allele is mostly prevalent in West Africa or in individuals with ancestry from this region. HbSC disease (caused by βS/βC) is a condition with generally milder hemolytic anemia and less frequent acute and chronic complications than SCA, although retinopathy and osteonecrosis (also known as bone infarction, in which bone tissue is lost owing to interruption of the blood flow) are common occurrences. The β3S allele combined with a null HBB allele (Hbβ0) that results in no protein translation causes HbSβ0-thalassaemia, a clinical syndrome indistinguishable from SCA except for the presence of microcytosis (a condition in which erythrocytes are abnormally small). The βS allele combined with a hypomorphic HBB allele (Hbβ+; with a decreased amount of normal β-globin protein) results in HbSβ+-thalassaemia, a clinical syndrome generally milder than SCA owing to low-level expression of normal HbA. Severe and moderate forms of HbSβ-thalassaemia are most prevalent in the eastern Mediterranean region and parts of India, whereas mild forms are common in populations of African ancestry.

One proposed strategy for treating SCD is to correct a mutant HBB gene back to encode wild-type beta-globin or force express wild-type beta-globin. For example, Park et al. (Park et al., Nucleic Acids Res. 2019 Sep. 5; 47(15):7955-7972.) used CRISPR/Cas9 targeting the βS allele and a short single-stranded oligonucleotide template to correct the sickle mutation in βS in hematopoietic stem and progenitor Cells (hHSPCs) from peripheral blood or bone marrow of SCD patients. The CRISPR treatment markedly increased normal HbA and reduced sickle cells.

An alternative approach may involve reversing Hb switching by suppressing beta-globing expression and/or enhancing gamma-globin expression via modifying the enhance and/or repressor of a beta-like globin gene. For example, when the GATA1-binding site of the BCL11A gene's enhancer was edited via the CRISPR/Cas9 system in SCD patient CD34⁺ HSCs, erythroid cells derived from such HSCs showed increased gamma-globin expression, and the sickling morphology was prevented (Wu et al., Nat Med. 2019 May; 25(5):776-783. Epub 2019 Mar. 25.). Similarly, targeting of the KLF1 gene via the CRISPR/Cas9 system in K562 cells also increased the HBG transcript levels and the HbF protein expression levels (Shariati et al., J Gene Med. 2016 October; 18(10):294-301.). Analogous observation was also made by other studies, such as Lamsfus-Calle et al., Sci Rep. 2020 Jun. 23; 10(1):10133.

Currently, several gene therapy strategies for treating β3-hemoglobinopathies are being tested in the clinic or are about to enter the clinical stage. For example, Vertex Pharmaceuticals and CRISPR Therapeutics recently tested safety and efficacy of their CTX001, autologous CD34+ human HSPCs modified via the CRISPR/Cas9 system targeting the erythroid-specific enhancer region of BCL11A, in subjects with transfusion-dependent β-thalassemia (TDT) or severe SCD (ClinicalTrials.gov Identifiers: NCT03655678 (CLIMB THAL-111) and NCT03745287 (CLIMB SCD-121); Frangonl et al., N Engl J Med 2021; 384:252-60.). CD34+ HSPCs were collected from patients by apheresis after mobilization with either filgrastim and plerixafor or plerixafor alone. CTX001 was manufactured by performing gene editing ex vivo using the CRISPR/Cas system on the CD34+ HSPCs. Patients received busulfan myeloablation, followed the infusion of CTX001. Bluebird bio has been testing their LentiGlobin (“bb1111”), autologous CD34+ HSCs transduced ex vivo with the recombinant lentiviral vector encoding βA-T87Q-globin, in patients with TDT or SCD (e.g., ClinicalTrials.gov Identifiers: NCT02140554 (HGB-206) and NCT04293185 (HGB-210)). However, trials have been suspended after finding cancer cases. All these strategies involve harvesting HSCs, modifying HSCs ex vivo, myeloablation, and putting the modified HSCs back into the patients, which creates various difficulties and disadvantages. The lengthy treatment processes not only require an extremely high cost and a dedicated facility but also impose patients a physiological burden. Furthermore, because of such issues, especially considering SCDs are more prevalent in regions with less access to high-quality healthcare such as West Africa (Kato et al., Nat Rev Dis Primers. 2018 Mar. 15; 4:18010.), these therapeutic strategies would unlikely solve the global health problem. The current disclosure provides a method that circumvents the need of HSC harvesting, myeloablation, and infusion of ex vivo edited HSCs.

The term “Sickle cell disease-associated gene” or “SCD-associated gene” as used herein refers to any genes and their mutant forms involved in or associated with the pathogenesis and/or pathology of SCD, including both coding and noncoding sequences (e.g., exons and introns) and regulatory elements for the gene such as promoters and enhancers. SCD-associated genes include genes involved in Hb switching. Non-limiting examples of SCD-associated genes include: HBB (e.g., the HbS variant), BCL11A, KLF1, SOX6, GATA1, NF-E4 (or NFE4), COUP-TF, NR2C1 (also known as TR2), NR2C2 (also known as TR4), genes encoding members of the MBD2 protein complex, IKZF1 (also known as Ikaros), genes encoding other members of PYR complex (CHD4, HDAC2, RBBP7, SMARCB1, SM4RCC1, SM4RCC2, SM4RCD1, and SMARCE1), and BRG1 (Sankaran et al., Br J Haematol. 2010 April; 149(2):181-94. Epub 2010 Mar. 1.), and also genes that directly or indirectly regulate expression thereof.

“Single-strand oligo DNA nucleotides” or “ssODN” as used herein refers to a short DNA fragment of a single strand comprising a particular polynucleotide sequence that may be useful for some of the embodiments disclosed herein. In one aspect, ssODN may be used as part of CRISPR/Cas-mediated gene editing disclosed herein and may function as a DNA template (may also referred to as a DNA repair template) to mediate a knock-in of a sequence of interest through the Cas9-mediated double-strand break site. Such a knock-in may be via homology-directed repair (HDR). In some embodiments, a ssODN may have homology to the strand that initiates repair in the direction of a desired modification. In some embodiments, a ssODN may comprise (i) a central region comprising one or more desired nucleic acids, sandwiched by (ii) a 5′ homology arm and (iii) a 3′ homology arm. Such a homology arm may comprise approximately 20-2500 nucleotides (nt). 5′ and 3′ homology arms often have the same or similar nucleotide lengths (e.g., 0 or 1 to 10 nt difference), but 5′ and 3′ homology arms that significantly differ in length may also be used as long as the ssODN mediate an intended gene repair. 5′ and/or 3′ homology arms may be 100% complementary to the corresponding sequence in the original DNA sequence before gene editing or may have one or more (a few) mutations (e.g., silent mutation) relative to the corresponding sequence in the original DNA sequence before gene editing. In some embodiments, ssODN may have one or more mutations at the PAM sequence (or its reverse (or antisense) sequence of to the PAM sequence, i.e., the opposite strand) and/or at one or more of the 5′-neighbouring bases of the PAM (or the 3′-neighbouring bases of the reverse (or antisense) sequence corresponding to the PAM). In some cases, such a mutation(s) helps prevent or reduce Cas-mediated cleavage of the ssODN itself or of a gene-edited DNA molecule. In some embodiments, a ssODN may comprise complementarity to the gRNA strand. In some embodiments, a ssODN may comprise a total length of approximately 40-5000 nucleotides (nt). As a DNA repair template, a double-stranded DNA template may also be used instead. In such a case, one of the strands of the template may comprise the same sequence as a desired ssODN and the other strand have a sequence complementary thereto.

The term “stem cell mobilization” as used herein refers to a process in which the movement of stem cells from the bone marrow into the blood is stimulated. In some embodiments, the stem cells mobilized may be HSCs and/or HSPCs. Exemplary agents that promote stem cell mobilization include G-CSF, GM-CSF, Plerixafor, and SCF (Hopman and DiPerio, Blood Rev 2014 January; 28(1): 31-40). Other exemplary agents that promote stem cell mobilization include but are not limited to CXCR4 antagonists (e.g., POL6326, BKT-140, TG-0054), CXCL12 neutralizers (e.g., NOX-A12), Sphingosine-1-phosphate (SIP) antagonists (e.g., SEW2871), vascular cell adhesion molecule-1/Very Late Antigen 4 (VCAM/VLA-4) inhibitors (e.g., BIO 5192), parathyroid hormone, protease inhibitors (e.g., Bortezomib), Grop (e.g., SB-251353), hypoxia inducible factor (HIF) stabilizers (e.g., FG-4497).

A “subject” as used herein, which may be interchangeably referred to as “patient”, “individual”, or “animal”, refers to a vertebrate including members of the mammalian species, such as canine, feline, lupine, mustela, rodent (racine, murine, etc.), equine, bovine, ovine, caprine, porcine species, and primates including humans. In specific embodiments, the subject is a human. In some embodiments, a subject may have or have a risk of developing a target disease. In specific embodiments, a subject may have or have a risk of developing SCD.

The term “target cell” or “host cell” as used herein refers to a cell in which the cargo of a TCV according to the present disclosure is intended to function. A TCV according to the present disclosure may be engineered to specifically carry its cargo in a target cell, for example by comprising one or more targeting moiety on the surface.

The term “target disease”, as used herein, which may be used interchangeably with “target disorder” or “target condition”, refers to a disease, disease, or condition that a TCV containing a cargo or a composition containing such a TCV according to the present disclosure is intended to treat, prevent, or ameliorate. A TCV according to the present disclosure may carry its cargo into a target cell, thereby altering a target gene or target gene expression and thus prevent, treat, or ameliorate a target disease.

The term “target gene” or “target gene of interest” as used herein is a gene (including the gene itself and in some cases a polynucleotide region that regulates the expression of the gene such as a promoter and/or an enhancer of the gene) whose sequence is to be altered (e.g., disrupted, partially or entirely removed, or partially or entirely replaced with an intended sequence, for example by a nuclease (such as Cas9) and a guide RNA) or whose expression is to be altered (e.g., reduced or diminished or, in some cases, completely abrogated, for example by a siRNA, shRNA, or miRNA) by a cargo of a TCV according to the present disclosure. In general, “target gene” may be any gene of interest in a target cell. The sequence of “target gene” encompasses the sense antisense strand sequences of the gene.

The term “target sequence” or “target polynucleotide sequence” as used herein is the sequence of a polynucleotide that a cargo of a TCV according to the present disclosure may interact with in a target cell to alter the target gene and/or target gene expression.

The term “therapeutically effective amount/dose” refers to the quantity of a TCV or a pharmaceutical composition comprising such a TCV or its cargo that is sufficient to provide a therapeutic effect (which may be based on, e.g., the number or percentage of target cells in which the intended target gene alteration occurred, the overall change in the target gene expression, the amelioration of one or more symptom, the number or percentage of target cells exhibiting an intended phenotype such as morphology, etc) upon administration to a subject.

The term “transfection competent vesicle” or “TCV” as used herein, in its broadest sense, encompasses any materials capable of carrying one or more cargoes, such as but not limited to a nucleic acid molecule (e.g., a DNA or a RNA) and/or a nucleic acid molecule complexed with a protein or peptide, into a cell. Examples of TCVs include but are not limited to: compounds, such as calcium phosphate, polycations, cationic lipids, phospholipids, organic and nonorganic polymers, dendrimers, organic and nonorganic nanoparticles and nanobeads, and any combinations thereof; lipid-based compositions capable of carrying a nucleic acid molecule, such as liposomes and lipid nanoparticles (LNPs); plasmids; virus-like particles (VLPs); and viral vectors, such as retroviral, lentiviral, and adenoviral vectors. In some embodiments, a TCV may comprise a targeting moiety (e.g., antibody or antibody fragment such as a Fab fragment), which allows the TCV to carry its cargo preferentially into a target cell. In some embodiments, such a targeting moiety may be specific to HSCs, HSCPs, MPPs, CMPs, MEPs, HPCs, erythroid progenitors (e.g., BFU-E, CFU-E), proerythroblasts, erythroblasts (basophilic erythroblasts, early erythroblasts (e.g., type I, type II), polychromatic erythroblasts, intermediate erythroblasts, acidophilic erythroblasts, late erythroblasts, normoblasts, or reticulocytes (before nucleus expulsion).

As used herein, the term “treat,” “treatment,” or “treating” generally refers to the clinical procedure for reducing or ameliorating the progression, severity, and/or duration of a disease or of a condition, or for ameliorating one or more conditions or symptoms (preferably, one or more discernible ones) of a disease. In specific embodiments, the effect of the “treatment” may be evaluated by the amelioration of at least one measurable physical parameter of a disease, resulting from the administration of one or more therapies. The parameter may be, for example, gene expression profiles, the number of disease-affected cells, the percentage or frequency of disease-affected cells among the cells of the same lineage, disease-associated marker levels, and/or the presence or absence or levels of certain cytokines or chemokines or other disease-associated molecules and may not necessarily discernible by the patient. In some embodiments “treat”, “treatment,” or “treating” may result in and/or be evaluated based on the inhibition of the progression of a disease, either physically by, e.g., stabilization of a discernible symptom, physiologically by, e.g., stabilization of a physical parameter, or both. In some embodiments the terms “treat”, “treatment” and “treating” refer to the reduction or stabilization of cancerous tissue or cells. Additionally, the terms “treat,” and “prevent” as well as words stemming therefrom, as used herein, do not necessarily imply 100% or complete cure or prevention. Rather, there are varying degrees of treatment effects or prevention effects of which one of ordinary skill in the art recognizes as having a potential benefit or therapeutic effect. In this respect, the inventive methods can provide any amount of any level of treatment or prevention effects of a disease in a mammal. Furthermore, the treatment or prevention provided by the inventive method can include treatment or prevention of one or more conditions or symptoms of the disease being treated or prevented. Also, for purposes herein, “prevention” can encompass delaying the onset of the disease, or a symptom or condition thereof.

As will be understood by one having ordinary skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While in the above set forth preferred construction, specific elements have been recited in order to adequately illustrate the principles of this invention, it will be apparent to those skilled in the art that alterations and modifications in the construction and arrangement of the system may be made without thereby departing from the spirit of said invention. Changes of form, of details of construction and materials may be made without thereby departing from the spirit of invention set forth, which shall be limited only by the scope of the appended claims/Examples are provided below to illustrate the present invention. These examples are not meant to constrain the present invention to any particular application or theory of operation.

### EXAMPLES

### Example 1: Preparation of Transfection Competent Vesicles (TCVs)

### Materials

1,2-Dioleyloxy-3-dimethylamino-propane (DODMA) was purchased from Cayman Chemical (Ann Arbor, MI). 1,2-dioleoyl-sn-glycero-3-phosphoethanolamine (DOPE) and 1,2-distearoyl-sn-glycero-3-phosphocholine (DSPC) were purchased from Avanti Polar Lipids (Alabaster, AL). Cholesterol was purchased from Sigma Aldrich (St. Louis, MO). All lipids were maintained as ethanol stocks.

### TCV Formation

Lipid components (ionizable cationic lipid, helper lipid, phospholipid, and cholesterol) were dissolved in ethanol at appropriate ratios to achieve a final concentration of about 20-35 mM total lipid. Unless otherwise noted, the lipid ratio of DODMA:DOPE:DSCP:cholesterol=20:30:10:40 mol % was used. An aqueous phase was prepared containing about 25 mM sodium acetate (approximately pH 4) buffer. The two solutions were combined via rapid-mixing. Specifically, the organic phase containing lipids was mixed with the aqueous phase through a T-junction mixer fabricated to meet the specifications of the PEEK Low Pressure Tee Assembly ( 1/16, 0.02 in thru hole, Part #P-712) at a final flow rate of about 20 m/min with about 1:3 organic:aqueous (v/v) ratio (Jeffs, Palmer, et al., Pharm Res. 2005; 22(3): 362-372.; Kulkarni et al., Nanoscale. 2017 Sep. 21; 9(36):13600-13609; Kulkarni et al., ACS Nano. 2018 May 22; 12(5):4787-4795.). The resulting suspension was dialyzed against 1000-fold volume of 25 mM sodium acetate (approximately pH 4) buffer to remove ethanol.

### Analysis of TCVs

Lipid concentrations were determined by assaying for the cholesterol content using a T-Cholesterol Assay Kit (Wako Chemicals, Mountain View, CA) and extrapolating total lipid concentration as described elsewhere (Chen et al., J Control Release. 2014 Dec. 28; 196:106-12.). Nucleic acid entrapment was determined using the RiboGreen Assay as previously described (Chen et al., J Control Release. 2014 Dec. 28; 196:106-12; Leung et al., J Phys Chem B. 2015 Jul. 16; 119(28):8698-706.).

### Example 2: Preparation of RNP

Recombinant Cas9 nuclease protein was obtained from IDT (San Jose, CA). Single-stranded gRNAs (sgRNAs) designed by Applicant were ordered from Synthego (Redwood City, CA). RNP formation was performed by combining a sgRNA solution of about 10 μM with a Cas9 solution of about 10 μM (at an approximately equimolar ratio between the total sgRNA and Cas9) and allowing to stand at room temperature for about 5 minutes prior to encapsulation of the RNP in TCVs.

### Example 3: Preparation of RNP-TCV with or without DNA Repair Template

### Encapsulation of RNP (No DNA Template)

An about 0.5-20 mM TCV solution (about pH 4) and an about 0.5-20 μM RNP solution (about pH7) were combined at a 467:1 to 5000:1 molar ratio (molar concentration for TCV is the concentration of total lipid components of the TCV). Typically, 8.33 μL of a 10 mM TCV solution and 10 μL of about 5 μM RNP were combined. The mixture was then allowed to incubate at room temperature for 5 minutes.

### Co-Encapsulation of RNP and DNA Template

An about 0.5-20 mM TCV solution (about pH 4), an about 0.5-20 μM RNP solution (about pH7), and an about 0.5-20 μM DNA template (e.g., ssODN) solution (about pH7) were combined at a 467:1:1 to 5000:1:1 molar ratio (molar concentration for TCV is the concentration of total lipid components of the TCV). Typically, 8.33 μL of a 10 mM TCV solution (molar of total lipid components) (about pH 4), 10 μL of a mixture containing about 5 μM RNP (about pH 7), and 5 μL of a 10 μM solution of ssODN (about pH 7) were combined. The mixture was then allowed to incubate at room temperature for 5 minutes.

Encapsulation of DNA Template Separate from RNP

An about 0.5-20 mM TCV solution (about pH 4) and an about 0.5-20 DNA template (e.g., ssODN) solution (about pH7) were combined at a 467:1 to 5000:1 molar ratio (molar concentration for TCV is the concentration of total lipid components of the TCV). Typically, 8.33 μL of a 10 mM TCV solution (molar of total lipid components) (about pH 4) and 5 μL of a 10 μM solution of ssODN (about pH 7) were combined. The mixture was then allowed to incubate at room temperature for 5 minutes.

### Example 4: Proof-of-Concept Gene Editing in HSCs, HSPCs, and/or Bone Marrow Cells

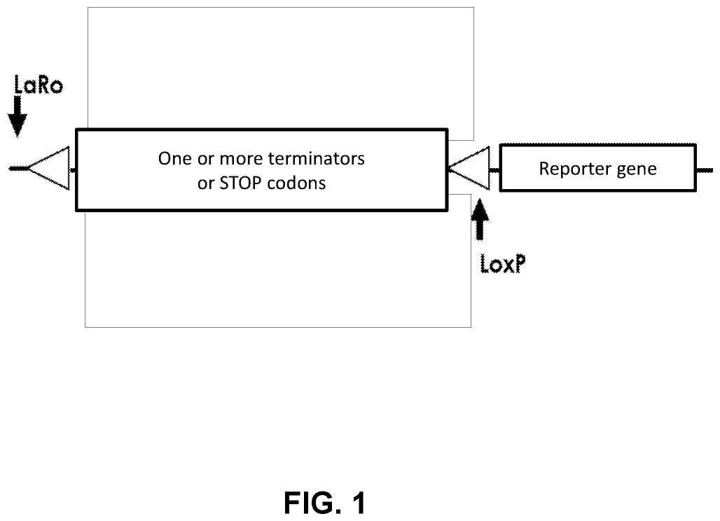

To first confirm that the TCVs according to the present disclosure are compatible with and are able to deliver a cargo to effect gene editing in HSCs, HSPCs, and/or bone marrow cells are harvested from a CRISPR/Cas gene editing reporter mouse (FIG. 1). For example, the femur and tibia will be harvested, and bone marrow cells will be collected by pushing saline through the marrow using a syringe and a needle, followed by several cycles of washing. For HSCs and/or HSPCs, CD34+ cells may be isolated from the collected bone marrow cells via anti-CD34 staining followed by FACS sorting or via a CD34+ cell magnetic isolation kit.

TCVs are generated according to Example 1. The TCV components of DODMA:DOPE:DSCP:cholesterol=20:30:10:40 may be used. Any of the gRNAs designed to effect interruption of the terminators and/or stop codons is complexed with Cas nuclease (e.g., Cas9, for example of Streptococcus pyogenes (SpCas9)) to generate RNPs according to Example 2. RNP encapsulation by the TCV will be performed as in Example 3.

The HSCs, HSPCs, and/or bone marrow cells are incubated with the RNP-TCV complex. Exemplary incubation protocol may be, for example, in Applicant's WO2020077007A1. Successful gene editing is confirmed via expression of the reporter gene, measured e.g., by flow cytometry and/or fluorescence microscopy.

### Example 5: Proof-of-Concept Gene Editing Via Intramarrow Injection in Reporter Mice

Next, Applicant will confirm that intramarrow injection of the pharmaceutical compositions according to the present disclosure allows the TCV to carry its cargo for gene editing into HSCs, HSPCs, and/or bone marrow cells and provide intended gene editing. The same RNP-TCV complex and the same reporter mouse as in Example 4 will be used.

For example, a composition comprising the RNP-TCV complex (comprising about 2700 pmol RNPs per mL) will be injected into the bone marrow (e.g., of the femur) of the reporter mice at up to about 50 μl per minute for 5, 10, 20, 30, or 60 minutes. One, two, three, five, seven, 14, or 30 days later, the whole blood will be harvested, mice will be sacrificed, and the bone marrow cells will be harvested.

The blood cells and bone marrow cells will be washed and stained with anti-CD34 antibody and analyzed for the reporter gene expression, e.g., by flow cytometry. Percentage of cells expressing the reporter gene among the total blood cells, total bone marrow cells, CD34+ cells in the blood, CD34+ cells in the bone marrow cells will be calculated to confirm successful gene editing in the cells.

### Example 6: Proof-of-Concept Gene Editing Via IV Injection Following Stem Cell Mobilization in Reporter Mice

Applicant will then confirm that injection of the pharmaceutical compositions according to the present disclosure to the peripheral circulation, following stem cell mobilization, allows the TCV to carry its cargo for gene editing into HSCs and/or HSPCs cells in the peripheral circulation and provide intended gene editing. The same RNP-TCV complex and the same reporter mouse as in Example 4 will be used.

Mice will receive G-CSF (Filgrastim) at a dose of about 10 μg/kg/day for 4 days. On day 4, plerixafor is also administered at a dose of about 0.24 mg/kg body weight. About 5 days after last plerixafor administration, a composition comprising the RNP-TCV complex (comprising about 2700 pmol RNPs per mL) will be IV injected to the reporter mice at up to about 50 μl per minute for 5, 10, 20, 30, or 60 minutes. One, two, three, five, seven, or 14 days later, the whole blood will be harvested, and mice will be sacrificed. The bone marrow cells may also be harvested and analyzed similarly, as some fraction of peripheral stem cells can return to the bone marrow.

The blood cells (and bone marrow cells) will be washed and stained with anti-CD34 antibody and analyzed for the expression of the reporter gene, e.g., by flow cytometry. Percentage of cells expressing the reporter gene among the total blood cells, (total bone marrow cells,) CD34+ cells in the blood, (CD34+ cells in the bone marrow cells) will be calculated to confirm successful gene editing in the cells.

### Example 7: SCD-Associated Gene Editing in SCD HSCs, HSPCs, or Bone Marrow Cells

To confirm that the gRNAs targeting SCD-associated genes according to the present disclosure are compatible with and are able to induce intended gene editing in HSCs, HSPCs, and/or bone marrow cells are harvested from mice (or another model animal) carrying at least one βS allele (SCD mice, for example, Noguchi et al., Blood Cells Mol Dis. November-December 2001; 27(6):971-7.). For example, the femur and tibia will be harvested, and bone marrow cells will be collected by pushing saline through the marrow using a syringe and a needle, followed by several cycles of washing. For HSCs and/or HSPCs, CD34+ cells may be isolated from the collected bone marrow cells via anti-CD34 staining followed by FACS sorting or via a CD34+ cell magnetic isolation kit. Alternatively, equivalent cells from SCD patients may be used.

TCVs are generated according to Example 1. The TCV components of DODMA:DOPE:DSCP:cholesterol=20:30:10:40 may be used. Any of the gRNAs designed to target a SCD-associated gene may be prepared. For example, for targeting the β3S allele, gRNA may comprise the target-complementary sequence of SEQ ID NO: 25, 45, 47, or 49; for targeting BCL11A, gRNA may comprise the target-complementary sequence of SEQ ID NO: 65, 67, or 69; for targeting KLF1, gRNA may comprise the target-complementary sequence of SEQ ID NO: 75 or 77; and for targeting HBG1 and/or HBG2, gRNA may comprise the target-complementary sequence of SEQ ID NO: 85. The gRNA is complexed with Cas nuclease (e.g., Cas9, for example of Streptococcus pyogenes (SpCas9)) to generate RNPs according to Example 2. For correcting a mutant HBB gene, a DNA repair template may also be prepared. For example, such a template may comprise any of SEQ ID NOS: 169-176 and 101-108. RNP (and optionally DNA template) encapsulation by the TCV will be performed as in Example 3.

The HSCs, HSPCs, and/or bone marrow cells are incubated with the RNP-TCV or RNP-DNA template-TCV complex. Exemplary incubation protocol may be, for example, in Applicant's WO2020077007A1. Successful gene editing is confirmed based on (i) the absence of the original gene and/or (ii) the presence of the corrected gene when a DNA template was used, using PCR.

### Example 8: SCD-Associated Gene Editing Via Intramarrow Injection in SCD Mice

Next, Applicant will confirm that intramarrow injection of the pharmaceutical compositions according to the present disclosure allows the TCV to carry it's CRISPR/Cas-effecting cargo for gene editing into HSCs, HSPCs, and/or bone marrow cells and provide gene editing (optionally including correction) to the SCD-associated gene(s). The same RNP-TCV or RNP-DNA template-TCV complex and the same SCD mice as in Example 7 (or mice of another strain, such as wild-type mice (with gRNAs designed to target the ortholog of the corresponding human SCD-associated gene) or humanized mice) will be used.

For example, a composition comprising the RNP-TCV or RNP-DNA template-TCV complex (comprising about 2700 pmol RNPs per mL) will be injected into the bone marrow (e.g., of the femur) of the SCD mice at up to about 50 μl per minute for 5, 10, 20, 30, or 60 minutes. One, two, three, five, seven, 14, or 30 days later, the whole blood will be harvested, mice will be sacrificed, and the bone marrow cells will be harvested and washed.

A portion of the blood cells and bone marrow cells will be stained with an anti-CD34 antibody to isolate CD34+ cells by sorting. Successful gene editing is confirmed based on (i) the absence of the original gene and/or (ii) the presence of the corrected gene when a DNA template was used, using PCR. Percentage of gene-edited cells among the total blood cells, total bone marrow cells, CD34+ cells in the blood, CD34+ cells in the bone marrow cells will be calculated to confirm successful gene editing in the cells. In some cases, the expression of HBG (gene and/or protein) and/or the amount of HbF will also be measured.

### Example 9: SCD-Associated Editing Via IV Injection Following Stem Cell Mobilization in SCD Mice

Applicant will then confirm that injection of the pharmaceutical compositions according to the present disclosure to the peripheral circulation, following stem cell mobilization, successfully lead to gene editing (optionally including correction) of SCD-associated genes in HSCs and/or HSPCs cells in the peripheral circulation. The same RNP-TCV or RNP-DNA template-TCV complex and the same SCD mice as in Example 7 will be used.

Mice will receive G-CSF (Filgrastim) at a dose of about 10 μg/kg/day for 4 days. On day 4, plerixafor is also administered at a dose of about 0.24 mg/kg body weight. About 5 days after last plerixafor administration, a composition comprising the RNP-TCV or RNP-DNA template-TCV complex (comprising about 2700 pmol RNPs per mL) will be IV injected to the reporter mice at up to about 50 μl per minute for 5, 10, 20, 30, or 60 minutes. One, two, three, five, seven, or 14 days later, the whole blood will be harvested, and mice will be sacrificed. The bone marrow cells may also be harvested and analyzed similarly, as some fraction of peripheral stem cells can return to the bone marrow.

The blood cells (and bone marrow cells) will be washed. A portion of the blood cells (and bone marrow cells) will be stained with an anti-CD34 antibody to isolate CD34+ cells by sorting. Successful gene editing is confirmed based on (i) the absence of the original gene and/or (ii) the presence of the corrected gene when a DNA template was used, using PCR. Percentage of gene-edited cells among the total blood cells, total bone marrow cells, CD34+ cells in the blood, CD34+ cells in the bone marrow cells will be calculated to confirm successful gene editing in the cells.

### Example 10: HBB Gene Correction Via Intramarrow Injection in SCD Mice

Applicant will further confirm that addition of a DNA repair template according to the present disclosure in the pharmaceutical composition successfully corrects a HBB gene by intramarrow injection.

TCVs are generated according to Example 1. The TCV components of DODMA:DOPE:DSCP:cholesterol=20:30:10:40 may be used. A gRNA may comprise the target-complementary sequence of SEQ ID NO: 25, 45, 47, or 49. The gRNA is complexed with Cas nuclease (e.g., Cas9, for example of Streptococcus pyogenes (SpCas9)) to generate RNPs according to Example 2. A ssODN according to the present disclosure (e.g., for correction to wildtype beta-globin-encoding sequence, any of SEQ ID NOS: 169-176 and 101-108 or a sequence complementary thereto) or a double stranded DNA template having such a ssODN sequence will be encapsulated in TCVs in a similar manner to Example 3 (template may be encapsulated in TCVs separately from RNPs or together with RNPs). Treatment of SCD mice will be performed in a similar manner to Example 8.

The blood cells and bone marrow cells will be harvested and washed. A portion of the blood cells and bone marrow cells will be stained with an anti-CD34 antibody to isolate CD34+ cells by sorting. Successful gene correction is confirmed based on (i) the absence of the original gene and/or (ii) the presence of the corrected sequence using PCR followed by sequencing. Percentage of gene-corrected cells among the total blood cells, total bone marrow cells, CD34+ cells in the blood, CD34+ cells in the bone marrow cells will be calculated to confirm successful gene correction in the cells.

### Example 11: HBB Gene Correction Via IV Injection Following Stem Cell Mobilization in SCD Mice

Applicant will further confirm that addition of a DNA repair template according to the present disclosure in the pharmaceutical composition successfully corrects a SCD-associated gene by IV injection following stem cell mobilization.

The pharmaceutical composition comprising the repair DNA template used in Example 10 will be used. Treatment of SCD mice will be performed in a similar manner to Example 9.

The blood cells (and bone marrow cells) will be washed. A portion of the blood cells (and bone marrow cells) will be stained with an anti-CD34 antibody to isolate CD34+ cells by sorting. Successful gene correction is confirmed based on (i) the absence of the original gene and/or (ii) the presence of the corrected sequence using PCR followed by sequencing. Percentage of gene-corrected cells among the total blood cells, total bone marrow cells, CD34+ cells in the blood, CD34+ cells in the bone marrow cells will be calculated to confirm successful gene correction in the cells.

### Example 12: BCL11A Gene Editing in Human Cells

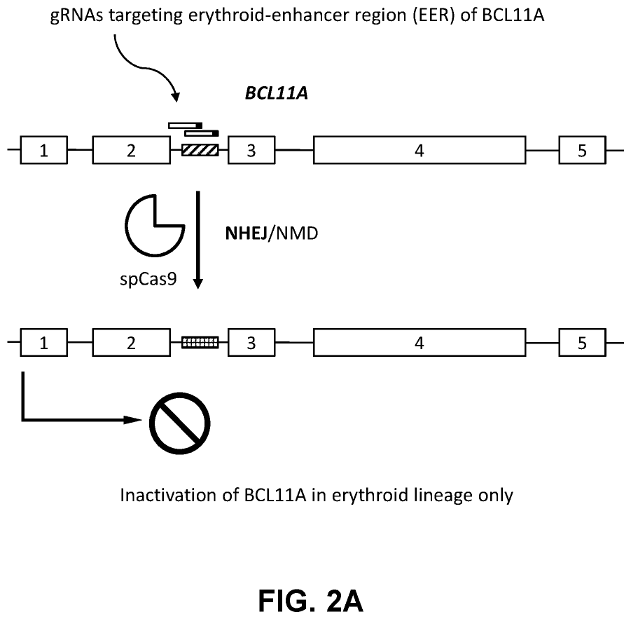

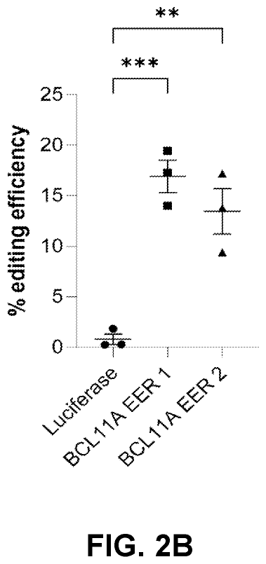

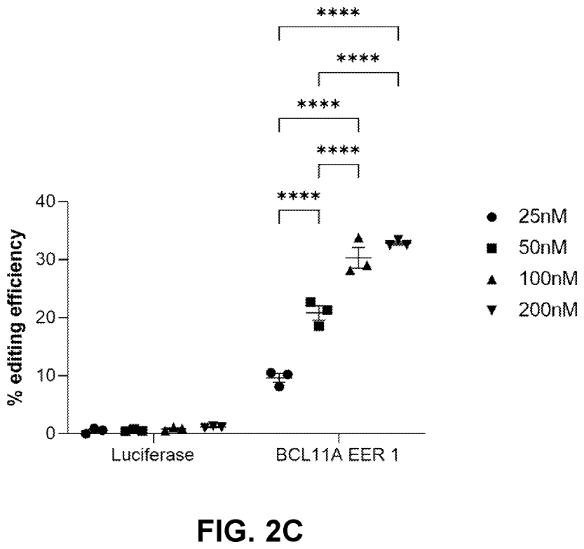

In this Example, the ability to disrupt the erythroid-enhancer region (EER) in intron 2 of BCL11A in human cells was tested. The editing strategy is visualized in FIG. 2A.

TCVs comprising DODMA:DOPE:DSCP:cholesterol=20:30:10:40 mol % were generated according to Example 1. RNPs for targeting the EER of BCL11A were generated by complexing a sgRNA comprising the target-complementary sequence of SEQ ID NO: 65 (complementary to region 1 of EER (EER 1) comprising SEQ ID NO: 64) or SEQ ID NO: 69 (complementary to region 2 of EER (EER 2) comprising SEQ ID NO: 68) with spCas9, as described in Example 2. Control RNPs for targeting luciferase were generated in the same manner using a sgRNA comprising the target-complementary sequence of SEQ ID NO: 55. RNPs were encapsulated by the TCVs as described in Example 3.

HEK293 cells were seeded at a density of 80,000 cells/mL on Day 0. Twenty-four hours after seeding (Day 1), the TCV-encapsulated RNPs targeting either luciferase or BCL11A EER were added to culture media at a final RNP concentration of 50 nM. Two hours after treatment (Day 1), culture media was changed, and cells were incubated for 22 hours prior to harvest. On Day 2, cells were harvested, and genomic DNA was extracted. A 788 base pair region of DNA flanking the two target sites of BCL11A EER was amplified using the forward and reverse primers of SEQ ID NOS: 61 and 62, respectively, and sent for Sanger sequencing. Percent editing efficiency at the target site was assessed using the Tracking of Indels by Decomposition (TIDE) analytical tool.

Exemplary results are shown in FIG. 2B. As shown in the graph, successful editing was observed at the respective target sites, BCL11A EER 1 and BCL11A EER 2.

### Example 13: Dose-Dependent BCL11A Gene Editing in Human Cells

In this Example, the effect of different doses of RNPs on editing of BCL11A EER in human cells was tested.

TCVs comprising DODMA:DOPE:DSCP:cholesterol=20:30:10:40 mol % were generated according to Example 1. RNPs for targeting the EER of BCL11A were generated by complexing a sgRNA comprising the target-complementary sequence of SEQ ID NO: 65 with spCas9, as described in Example 2. Control RNPs for targeting luciferase were generated in the same manner using a sgRNA comprising the target-complementary sequence of SEQ ID NO: 55. RNPs were encapsulated by the TCVs as described in Example 3.

HEK293 cells were seeded at a density of 80,000 cells/mL on Day 0. Twenty-four hours after seeding (Day 1), the TCV-encapsulated RNPs targeting either luciferase or BCL11A EER 1 were added to culture media at a final RNP concentration of either 25, 50, 100 or 200 nM (same stoichiometry of TCV:RNP as in Example 12 but added at increasing volumes). Two hours after treatment (Day 1), culture media was changed, and cells were incubated for 46 hours prior to harvest (Day 3). On Day 3, cells were harvested, and genomic DNA was extracted. A 788 base pair region of DNA flanking the BCL11A EER 1 target site was amplified and sequenced as described in Example 12. Percent editing efficiency at the target site was assessed using the TIDE analytical tool.

Exemplary results are shown in FIG. 2C. As shown in the graph, dose-dependent editing was observed at the target site (BCL11A EER 1).

### Example 14: Gene Editing in the BCL11A-Binding Site in the Promoter Region of HBG1 and HBG2 in Human Cells

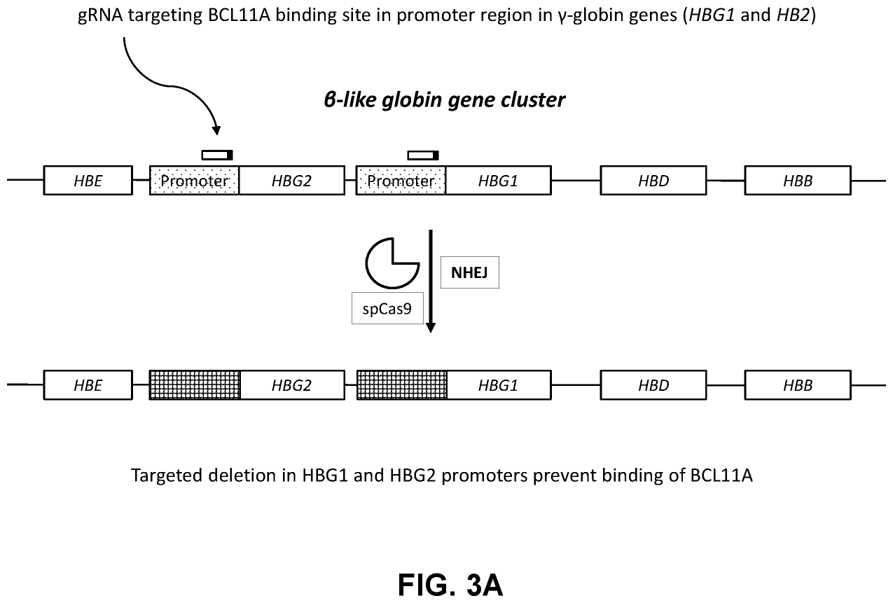

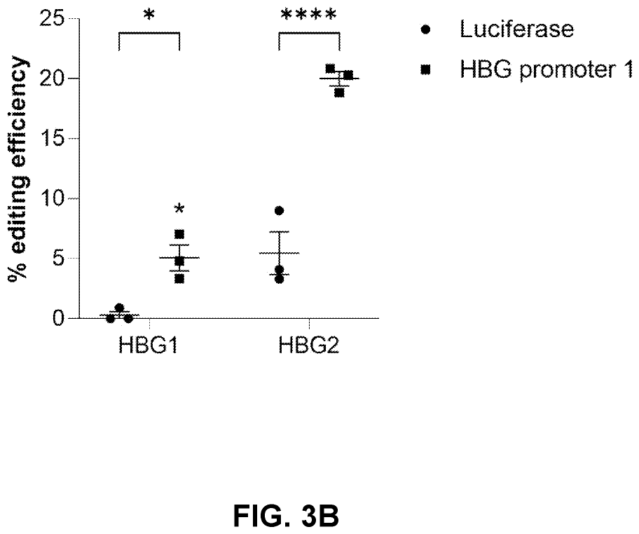

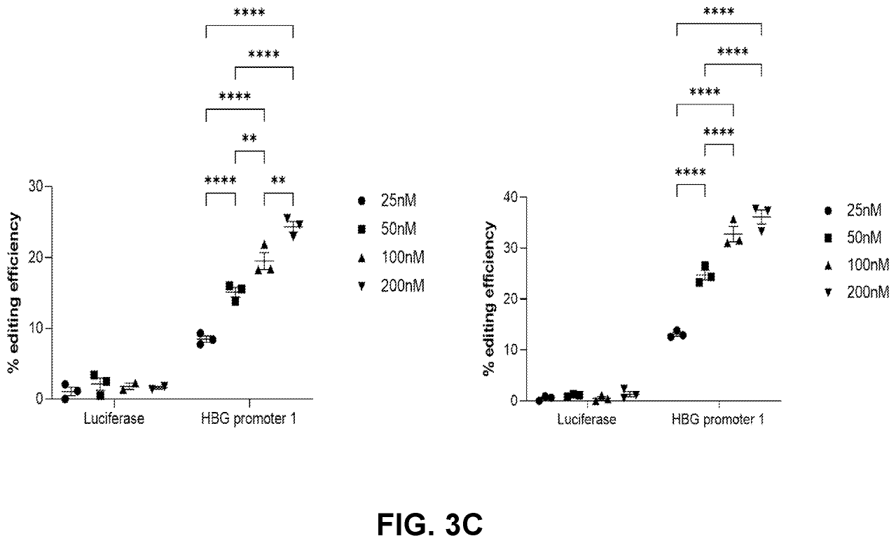

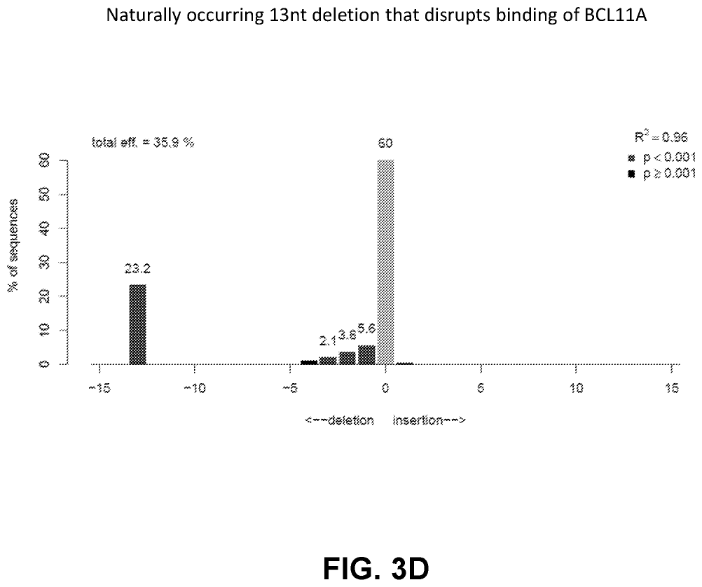

In this Example, the ability to disrupt the BCL11A-binding site in the promoter region of HBG1 and HBG2 in human cells was tested. The editing strategy is visualized in FIG. 3A.

TCVs comprising DODMA:DOPE:DSCP:cholesterol=20:30:10:40 mol % were generated according to Example 1. RNPs for targeting the BCL11A-binding site in the promoter region of HBG1 and HBG2 were generated by complexing a sgRNA comprising the target-complementary sequence of SEQ ID NO: 85 (complementary to the promoter region comprising SEQ ID NO: 84, designed to hybridize to both the BCL11A-binding promoter region of HBG1 and the BCL11A-binding promoter region of HBG2) with spCas9, as described in Example 2. Control RNPs for targeting luciferase were generated in the same manner using a sgRNA comprising the target-complementary sequence of SEQ ID NO: 55. RNPs were encapsulated by the TCVs as described in Example 3.

HEK293 cells were seeded at a density of 80,000 cells/mL on Day 0. Twenty-four hours after seeding (Day 1), TCV-encapsulated RNP targeting either luciferase or HBG1 and HBG2 promoter regions were added to culture media at a final RNP concentration of 50 nM. Two hours after treatment (Day 1), culture media was changed, and cells were incubated for 22 hours prior to harvest (Day 2). On Day 2, cells were harvested, and genomic DNA was extracted. A DNA region flanking the target HBG promoter site of HBG1 was amplified using the forward and reverse primers of SEQ ID NOS: 81 and 82, respectively, and sent for Sanger sequencing. A DNA region flanking the target HBG promoter site of HBG2 was amplified using the forward and reverse primers of SEQ ID NOS: 91 and 92, respectively, and sent for Sanger sequencing. Percent editing efficiency at HBG1 and HBG2 promoters was assessed using the TIDE analytical tool.

Exemplary results are shown in FIG. 3B. As shown in the graph, successful editing was observed in both of the target sites (the promoter of HBG1 and the promoter of HBG2).

### Example 15: Dose-Dependent Gene Editing in the BCL11A-Binding Site in the Promoter Region of HBG1 and HBG2 in Human Cells

In this Example, the effect of different doses of RNPs on editing of the BCL11A-binding site in the promoter region of HBG1 and HBG2 in human cells was tested.

TCVs comprising DODMA:DOPE:DSCP:cholesterol=20:30:10:40 mol % were generated according to Example 1. RNPs for targeting the BCL11A-binding site in the promoter region of HBG1 and HBG2 were generated by complexing a sgRNA comprising the target-complementary sequence of SEQ ID NO: 85 with spCas9, as described in Example 2. Control RNPs for targeting luciferase were generated in the same manner using a sgRNA comprising the target-complementary sequence of SEQ ID NO: 55. RNPs were encapsulated by the TCVs as described in Example 3.

HEK293 cells were seeded at a density of 80,000 cells/mL on Day 0. Twenty-four hours after seeding (Day 1), the TCV-encapsulated RNP targeting either luciferase or HBG1 and HBG2 promoter regions were added to culture media at a final RNP concentration of either 25, 50, 100 or 200 nM (same stoichiometry of TCV:RNP as in Example 14 but added at increasing volumes). Two hours after treatment (Day 1), culture media was changed, and cells were incubated for 46 hours prior to harvest (Day 3). On Day 3, cells were harvested, and genomic DNA was extracted. DNA regions flanking the target HBG promoter site of HBG1 or of HBG2 were amplified and sequenced as described in Example 14. Percent editing efficiency at HBG1 and HBG2 promoters and editing events were assessed using the TIDE analytical tool.

Exemplary editing efficiency results are shown in FIG. 3C. As shown in the graph, dose-dependent editing was observed in both of the target sites (the promoter of HBG1 and the promoter of HBG2). A representative histogram from the TIDE analysis showing distribution of specific editing events following treatment with the TCV-encapsulated RNP targeting the promoter region of HBG1 and HBG2 at 200 nM is shown in FIG. 3D. As shown in the graph, the most common editing event was a 13-nucleotide deletion, which is one known to be a naturally occurring mutation that leads to hereditary persistence of fetal hemoglobin, that disrupts the BCL11A-binding site within the HBG promoter. The 13-nucleotide deletion occurred at a frequency higher than would be expected by chance, as indicated by the p value of <0.001 calculated by TIDE.

### Example 16: Gene Editing in HBB Exon 1 in Human Cells

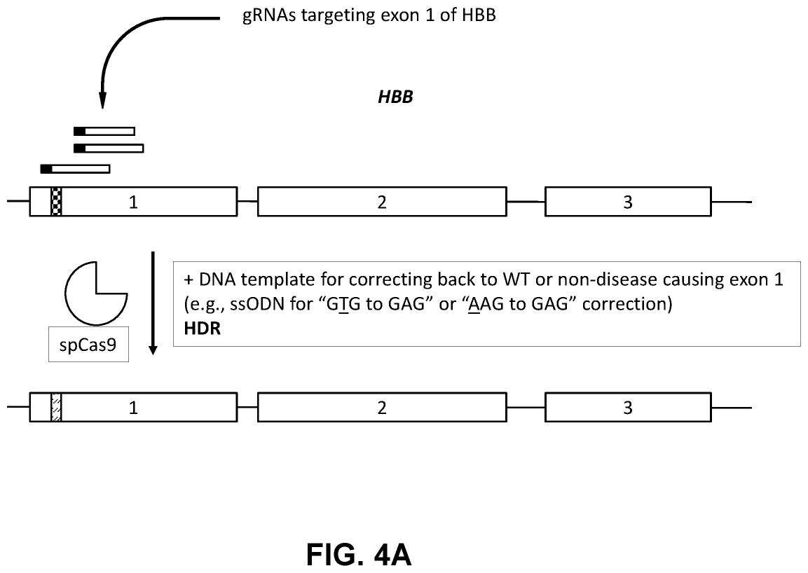

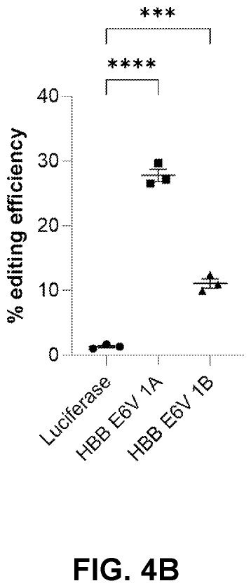

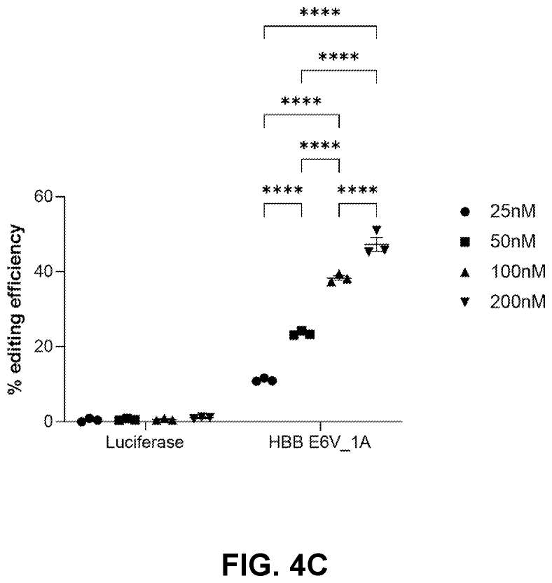

In this Example, the ability to edit or disrupt HBB exon 1 in human cells was tested. HBB exon 1 is the exon which contains the E-to-V mutation in the disease-causing HbS and HbC variants. Editing of HBB exon 1 as in this Example may be used as part of the gene correction strategy visualized in FIG. 4A.

TCVs comprising DODMA:DOPE:DSCP:cholesterol=20:30:10:40 mol % were generated according to Example 1. RNPs for targeting HBB exon 1 were generated by complexing a sgRNA comprising the target-complementary sequence of SEQ ID NO: 45 (complementary to HBB exon 1 region A (“E6V 1A”) comprising SEQ ID NO: 44) or SEQ ID NO: 47 (complementary to HBB exon 1 region B (“E6V 1B”) comprising SEQ ID NO: 46) with spCas9, as described in Example 2. Control RNPs for targeting luciferase were generated in the same manner using a sgRNA comprising the target-complementary sequence of SEQ ID NO: 55. RNPs were encapsulated by the TCVs as described in Example 3.

HEK293 cells were seeded at a density of 80,000 cells/mL on Day 0. Twenty-four hours after seeding (Day 1), TCV-encapsulated RNP targeting either luciferase, HBB E6V 1A, or HBB E6V 1B were added to culture media at a final RNP concentration of 50 nM. Two hours after treatment (Day 1), culture media was changed, and cells were incubated for 22 hours prior to harvest (Day 2). On Day 2, cells were harvested, and genomic DNA was extracted. A 746 base-pair region of DNA flanking the HBB E6V 1A and 1B target sites were amplified using the forward and reverse primers of SEQ ID NOS: 41 and 42, respectively, and sent for Sanger sequencing. Percent editing efficiency at the target sites was assessed using the TIDE analytical tool.

Exemplary results are shown in FIG. 4B. As shown in the graph, successful editing was observed at the respective target sites, HBB E6V 1A and HBB E6V 1B.

### Example 17: Dose-Dependent Gene Editing in HBB Exon 1 in Human Cells

In this Example, the effect of different doses of RNPs on editing of HBB exon 1 in human cells was tested.

TCVs comprising DODMA:DOPE:DSCP:cholesterol=20:30:10:40 mol % were generated according to Example 1. RNPs for targeting HBB exon 1 were generated by complexing a sgRNA comprising the target-complementary sequence of SEQ ID NO: 45 (complementary to HBB E6V 1A) with spCas9, as described in Example 2. Control RNPs for targeting luciferase were generated in the same manner using a sgRNA comprising the target-complementary sequence of SEQ ID NO: 55. RNPs were encapsulated by the TCVs as described in Example 3.

HEK293 cells were seeded at a density of 80,000 cells/mL on Day 0. Twenty-four hours after seeding (Day 1), TCV-encapsulated RNP targeting either luciferase or HBB exon 1 were added to culture media at a final RNP concentration of either 25, 50, 100 or 200 nM (same stoichiometry of TCV:RNP as in Example 16 but added at increasing volumes). Two hours after treatment (Day 1), culture media was changed, and cells were incubated for 46 hours prior to harvest (Day 3). On Day 3, cells were harvested, and genomic DNA was extracted. A 746 base-pair region of DNA flanking the HBB E6V 1A target site was amplified and sequenced as described in Example 16. Percent editing efficiency at the target site was assessed using the TIDE analytical tool.

Exemplary results are shown in FIG. 4C. As shown in the graph, dose-dependent editing was observed at the target site (HBB E6V 1A).

### Example 18: Proof-of-Concept Gene Correction in HBB Exon 1 in Human Cells

HBB exon 1 is the exon which contains the E-to-V mutation in the disease-causing HbS and HbC variants. To confirm the ability of providing gene correction at the nucleic acid position corresponding to the E-to-V mutation, in this Example, a E-to-V mutation will be introduced into human cells possessing WT HBB.

TCVs comprising DODMA:DOPE:DSCP:cholesterol=20:30:10:40 mol % are generated according to Example 1. RNPs for targeting HBB exon 1 are generated by complexing a sgRNA comprising the target-complementary sequence of SEQ ID NO: 45, 47, or 49 with spCas9, as described in Example 2. Control RNPs for targeting luciferase may be generated in the same manner using a sgRNA comprising the target-complementary sequence of SEQ ID NO: 55. RNPs are encapsulated by the TCVs as described in Example 3. A ssDNA such as one comprising any of SEQ ID NOS: 181-184 or a sequence complementary thereto or a double stranded DNA template having such a ssODN sequence will be encapsulated in TCVs in a similar manner to Example 3 (such a template may be encapsulated in TCVs separately from RNPs or together with RNPs).

HEK293 cells are seeded (e.g., at a density of 80,000 cells/mL) on Day 0. For example, twenty-four hours after seeding (Day 1), the RNP-DNA template-TCV complex (or a combination of the RNA-TCV complex and the DNA template-TCV complex) is added to culture media (e.g., at a final RNP concentration of 50 nM). For example, two hours after treatment (Day 1), culture media is changed and cells are incubated (e.g., for 22 hours prior to harvest (Day 2)). For example, on Day 2, cells are harvested, and genomic DNA is extracted. For example, a 746 base-pair region of DNA flanking the HBB target sites is amplified using the forward and reverse primers of SEQ ID NOS: 41 and 42, respectively, and sent for sequencing to confirm gene correction as intended. Percent editing efficiency at the target sites is assessed using the TIDE analytical tool.

### Example 19: SCD-Associated Editing Via Intramarrow Injection in Reporter Mice

That intramarrow injection of the pharmaceutical compositions according to the present disclosure allows the TCV to carry it's CRISPR/Cas-effecting cargo to facilitate gene editing into HSCs, HSPCs, and/or bone marrow cells and provide for effective gene editing (optionally including correction) to the SCD-associated gene(s) can be further confirmed, e.g., using methods disclosed in the present example. In particular this can be confirmed using the same reporter mouse as in Example 4.

In such experiments the same reporter gene targeting RNP as in Example 4 and a BCL11A-targeting RNP (e.g., targeting the mouse ortholog of BCL11A) will be prepared and both RNPs will be encapsulated in TCVs as described in Example 3. For example, a composition comprising the RNP-TCV or RNP-DNA template-TCV complex (comprising about 2700 pmol RNPs per mL) will be injected into the bone marrow (e.g., of the femur) of the reporter mice at up to about 50 μl per minute for 5, 10, 20, 30, or 60 minutes. One, two, three, five, seven, 14, or 30 days later, the whole blood will be harvested, mice will be sacrificed, and the bone marrow cells will be harvested and washed.

A portion of the blood cells and bone marrow cells will be stained with an anti-CD34 antibody to isolate CD34+ cells by sorting. Reporter gene-positive cells may be further sorted. Successful gene editing is confirmed based on the absence of the original gene (the mouse ortholog of human BCL11A), using PCR. Percentage of gene-edited cells among the total blood cells, total bone marrow cells, CD34+ cells in the blood, CD34+ cells in the bone marrow cells will be calculated to confirm successful gene editing in the cells. The expression of HBG (transcript by qPCR and/or protein) in the cell will also be measured. Additionally, the amount of HbF and/or the HbF/HbA ratio in the blood will also be measured by HPLC.

### Example 20: Gene Editing in the BCL11A Gene and the BCL11A-Binding Site in the Promoter Region of HBG1 and HBG2 in Human Erythroid Progenitor Cell Lines

The ability to disrupt the EER in intron 2 of BCL11A and the BCL11A-binding site in the promoter region of HBG1 and HBG2 in human erythroid progenitor cells (e.g., HUDEP-2 cells) according to the present invention can further be confirmed, e.g., using methods disclosed in the present example. The editing strategy is visualized in FIG. 2A.

TCVs comprising DODMA:DOPE:DSCP:cholesterol=20:30:10:40 mol % will be generated according to Example 1. RNPs for targeting the EER of BCL11A will be generated by complexing a sgRNA comprising the target-complementary sequence of SEQ ID NO: 65 (complementary to region 1 of EER (EER 1) comprising SEQ ID NO: 64) or SEQ ID NO: 69 (complementary to region 2 of EER (EER 2) comprising SEQ ID NO: 68) with spCas9, as described in Example 2. RNPs for targeting the BCL11A-binding site in the promoter region of HBG1 and HBG2 will be generated by complexing a sgRNA comprising the target-complementary sequence of SEQ ID NO: 85 (complementary to the promoter region comprising SEQ ID NO: 84, designed to hybridize to both the BCL11A-binding promoter region of HBG1 and the BCL11A-binding promoter region of HBG2) with spCas9, as described in Example 2. Control RNPs for targeting luciferase will be generated in the same manner using a sgRNA comprising the target-complementary sequence of SEQ ID NO: 55. RNPs will be encapsulated by the TCVs as described in Example 3.

Human erythroid progenitor cells (e.g., HUDEP-2 cells) will be seeded on Day 0. On Day 1, the TCV-encapsulated RNPs will be added to culture media at a final RNP concentration of 50 nM. On Day 1, culture media will be changed, and cells will be incubated for about 48 hours prior to harvest. On Day 3, cells will be harvested, and genomic DNA will be extracted. A 788 base pair region of DNA flanking the two target sites of BCL11A EER will be amplified using the forward and reverse primers of SEQ ID NOS: 61 and 62, respectively, and will be sent for Sanger sequencing. A DNA region flanking the target HBG promoter site of HBG1 was amplified using the forward and reverse primers of SEQ ID NOS: 81 and 82, respectively. A DNA region flanking the target HBG promoter site of HBG2 will be amplified using the forward and reverse primers of SEQ ID NOS: 91 and 92, respectively. Amplified DNAs will be sent for Sanger sequencing. Percent editing efficiency at each target site will be assessed using the TIDE analytical tool. The expression of BCL11A and HBG (transcript by qPCR and/or protein) in the cell will be measured. Additionally, the amount of HbF and/or the HbF/HbA ratio in the blood will also be measured by HPLC.

### Example 21: Gene Editing in HBB Exon 1 in SCD Patient-Derived Lymphoblastoid Cells

The ability to edit or disrupt HBB exon 1 in human SCD patient-derived lymphoblastoid cells (e.g., GM16265 lymphoblastoid cells homozygous for the E6V mutation in HBB) according to the present invention can additionally be confirmed, e.g., using methods disclosed in the present example. The gene editing may be used as part of the gene correction strategy visualized in FIG. 4A.

TCVs comprising DODMA:DOPE:DSCP:cholesterol=20:30:10:40 mol % will be generated according to Example 1. RNPs for targeting HBB exon 1 will be generated by complexing a sgRNA comprising the target-complementary sequence of SEQ ID NO: 25 (complementary to HBB exon 1 region comprising SEQ ID NO: 24), the target-complementary sequence of SEQ ID NO: 45 (complementary to HBB exon 1 region A (“E6V 1A”) comprising SEQ ID NO: 44), SEQ ID NO: 47 (complementary to HBB exon 1 region B (“E6V 1B”) comprising SEQ ID NO: 46), or SEQ ID NO: 49 (complementary to HBB exon 1 region B (“E6V 1B”) comprising SEQ ID NO: 48) with spCas9, as described in Example 2. Control RNPs for targeting luciferase will be generated in the same manner using a sgRNA comprising the target-complementary sequence of SEQ ID NO: 55. RNPs will be encapsulated by the TCVs as described in Example 3.

Cells will be seeded on Day 0. On Day 1, TCV-encapsulated RNP targeting either luciferase or HBB will be added to culture media at a final RNP concentration of 50 nM. On Day 1, culture media was changed, and cells were incubated for about 48 hours prior to harvest (Day 2). On Day 2, cells will be harvested, and genomic DNA will be extracted. A DNA fragment flanking the target site (e.g., flanking the HBB E6V 1A and 1B target sites) will be amplified using primers (e.g., the forward and reverse primers of SEQ ID NOS: 41 and 42, respectively) and sent for Sanger sequencing. Percent editing efficiency at the target sites will be assessed using the TIDE analytical tool.

### Example 22: Gene Correction in HBB Exon 1 in SCD Patient-Derived Lymphoblastoid Cells

The ability to correct the E6V mutation in HBB exon 1 in human SCD patient-derived lymphoblastoid cells (e.g., GM16265 lymphoblastoid cells homozygous for the E6V mutation in HBB) according to the invention can also be confirmed, e.g., using methods disclosed in the present example. The gene correction strategy is visualized in FIG. 4A.

TCVs comprising DODMA:DOPE:DSCP:cholesterol=20:30:10:40 mol % are generated according to Example 1. RNPs for targeting HBB exon 1 will be generated by complexing a sgRNA comprising the target-complementary sequence of SEQ ID NO: 25 (complementary to HBB exon 1 region comprising SEQ ID NO: 24), the target-complementary sequence of SEQ ID NO: 45 (complementary to HBB exon 1 region A (“E6V 1A”) comprising SEQ ID NO: 44), SEQ ID NO: 47 (complementary to HBB exon 1 region B (“E6V 1B”) comprising SEQ ID NO: 46), or SEQ ID NO: 49 (complementary to HBB exon 1 region B (“E6V 1B”) comprising SEQ ID NO: 48) with spCas9, as described in Example 2. Control RNPs for targeting luciferase may be generated in the same manner using a sgRNA comprising the target-complementary sequence of SEQ ID NO: 55. A ssDNA for correcting the E6V mutation back to wild-type HBB-encoding sequence such as one comprising any of SEQ ID NOS: 101-108 and 169-176 or a sequence complementary thereto or a double stranded DNA template having such a ssODN sequence will be encapsulated in TCVs in a similar manner to Example 3 (such a template may be encapsulated in TCVs separately from RNPs or together with RNPs).

Cells will be seeded (e.g., at a density of 80,000 cells/mL) on Day 0. On Day 1, the RNP-DNA template-TCV complex (or a combination of the RNA-TCV complex and the DNA template-TCV complex) will be added to culture media (e.g., at a final RNP concentration of 50 nM). For example, two hours after treatment (Day 1), culture media is changed and cells will be incubated (e.g., for 48 hours prior to harvest (Day 3)). For example, on Day 3, cells will be harvested, and genomic DNA will be extracted. A DNA fragment flanking the target site (e.g., flanking the HBB E6V 1A and 1B target sites) will be amplified using primers (e.g., the forward and reverse primers of SEQ ID NOS: 41 and 42, respectively) and sent for Sanger sequencing. Percent editing efficiency at the target sites (e.g., templated mutations as well as non-templated indels around the cut site) will be assessed using the TIDE analytical tool.

### APPENDIX: AMINO ACID AND NUCLEIC ACID SEQUENCES

HBB wild type:
Protein sequence
Human hemoglobin subunit beta (″beta-globin″, ″β-globin″, or ″HBB″), wild-type
SEQ ID NO: 1

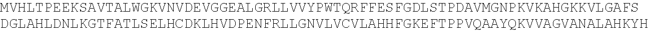

Nucleic acid sequence

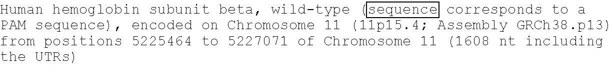

SEQ ID NO: 11

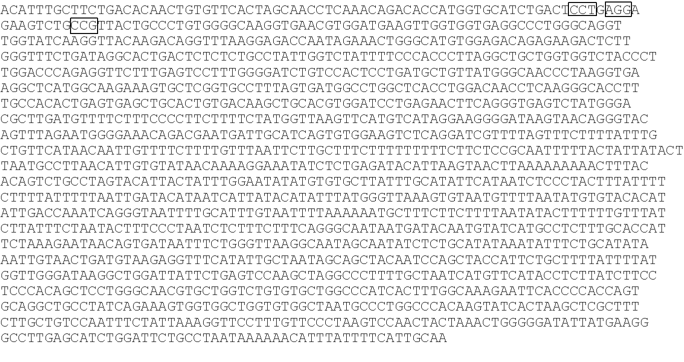

Nucleic acid sequence

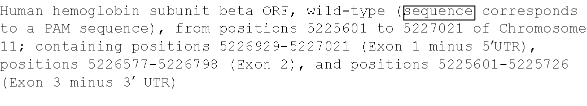

SEQ ID NO: 12

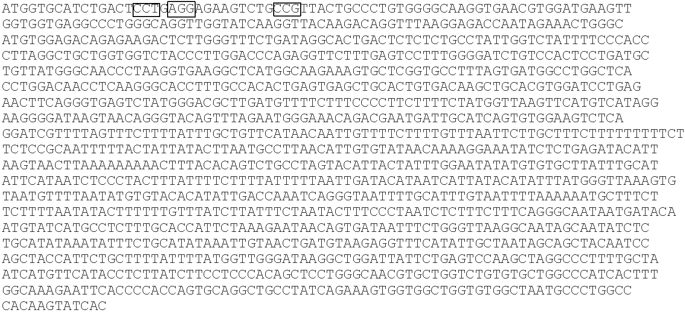

Nucleic acid sequence

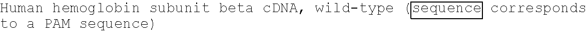

SEQ ID NO: 13

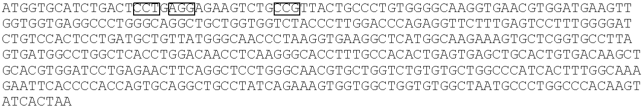

HBB HbS variant:
Protein sequence
Human hemoglobin subunit beta, Sickle Hb (HbS) variant
SEQ ID NO: 2

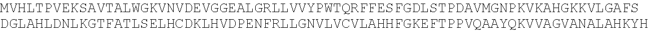

Nucleic acid sequence

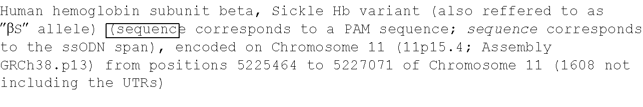

SEQ ID NO: 21

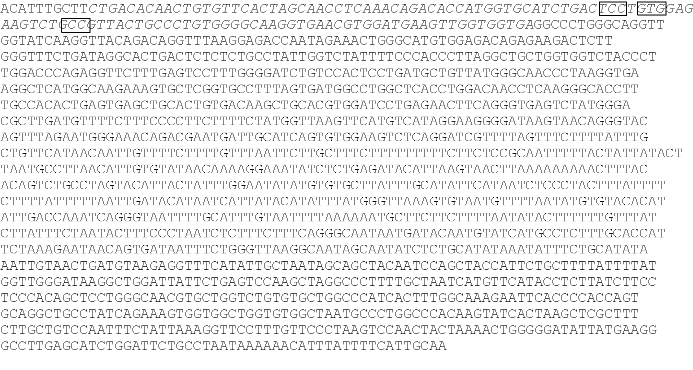

Nucleic acid sequence

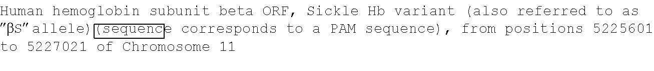

SEQ ID NO: 22

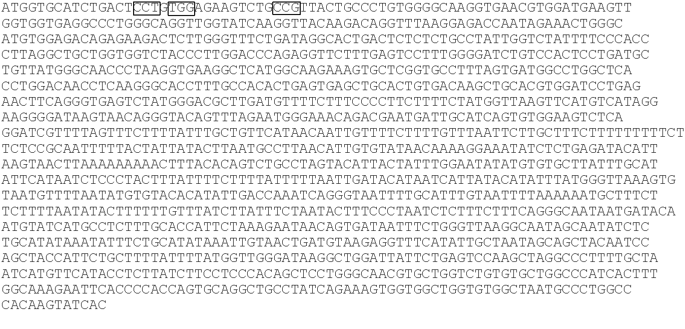

Nucleic acid sequence

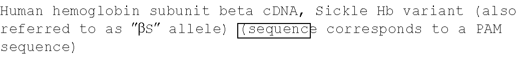

SEQ ID NO: 23

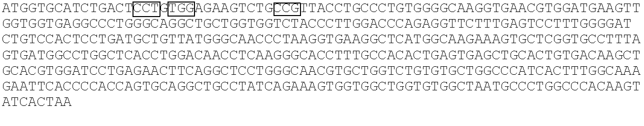

Nucleic acid sequence
Human HBB HbS variant CRISPR/Cas target sequence 1
SEQ ID NO: 24

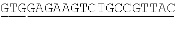

Nucleic acid sequence
gRNA target-complementary sequence, complementary to SEQ ID NO: 24
SEQ ID NO: 25

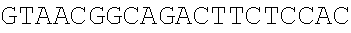

HBB HbC variant:
Protein sequence
Human hemoglobin subunit beta, Hb C (HbC) variant
SEQ ID NO: 3

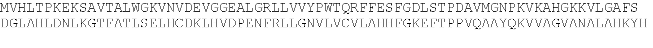

Nucleic acid sequence

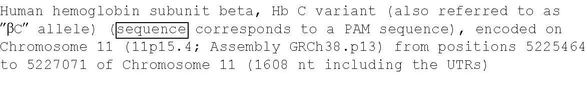

SEQ ID NO: 31

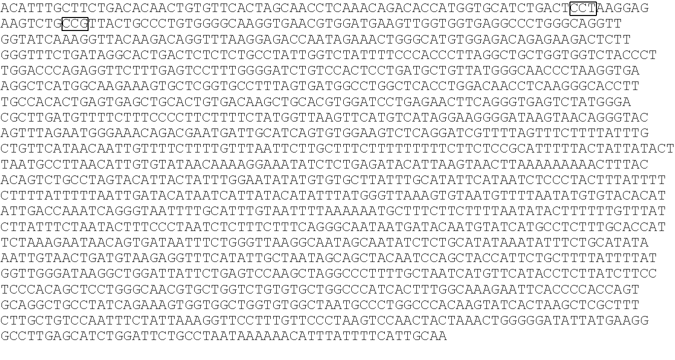

Nucleic acid sequence

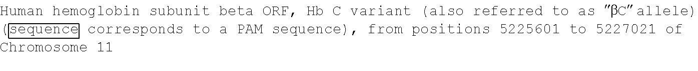

SEQ ID NO: 32

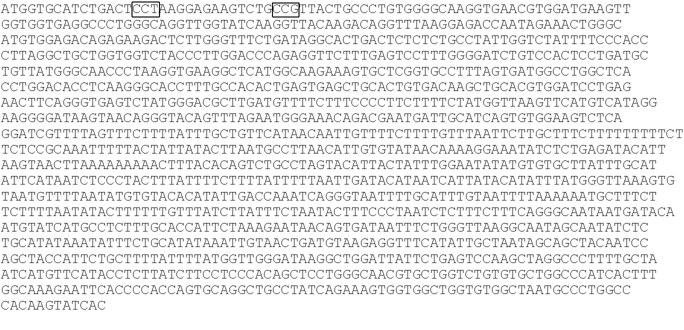

Nucleic acid sequence

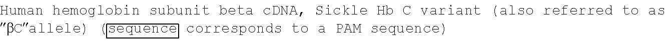

SEQ ID NO: 33

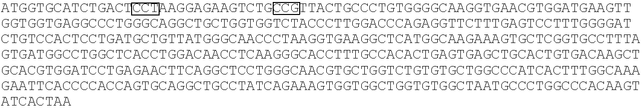

HBB exon 1 (any appropriate variant):
Nucleic acid sequence
Human HBB exon 1 forward primer
SEQ ID NO: 41
TGCTTACCAAGCTGTGATTCC
Nucleic acid sequence
Human HBB exon 1 reverse primer
SEQ ID NO: 42
CACTCAGTGTGGCAAAGGTG
Nucleic acid sequence
Human HBB exon 1 CRISPR/Cas target sequence 1
SEQ ID NO: 44
TTACTGCCCTGTGGGGCAAG
Nucleic acid sequence
gRNA target-complementary sequence, complementary to SEQ ID NO: 44
SEQ ID NO: 45
CTTGCCCCACAGGGCAGTAA
Nucleic acid sequence
Human HBB exon 1 CRISPR/Cas target sequence 2
SEQ ID NO: 46
TTACTGCCCTGTGGGGCA
Nucleic acid sequence
gRNA target-complementary sequence, complementary to SEQ ID NO: 46
SEQ ID NO: 47
TGCCCCACAGGGCAGTAA
Nucleic acid sequence
Human HBB exon 1 CRISPR/Cas target sequence 3
SEQ ID NO: 48
CAGGAGTCAGATGCACCATG
Nucleic acid sequence
gRNA target-complementary sequence, complementary to SEQ ID NO: 48
SEQ ID NO: 49
CATGGTGCATCTGACTCCTG

BCL11A:
Protein sequence
Human BAF chromatin remodeling complex sunbunit BCL11A (″BCL11A″)
SEQ ID NO: 6
MSRRKQGKPQHLSKREFSPEPLEAILTDDEPDHGPLGAPEGDHDLLTCGQCQMNFPLGDILIFIEHKRKQCNGSLCL
EKAVDKPPSPSPIEMKKASNPVEVGIQVTPEDDDCLSTSSRGICPKQEHIADKLLHWRGLSSPRSAHGALIPTPGMS
AEYAPQGICKDEPSSYTCTTCKQPFTSAWFLLQHAQNTHGLRIYLESEHGSPLTPRVGIPSGLGAECPSQPPLHGIHI
ADNNPFNLLRIPGSVSREASGLAEGRFPPTPPLFSPPPRHHLDPHRIERLGAEEMALATHHPSAFDRVLRLNPMAM
EPPAMDFSRRLRELAGNTSSPPLSPGRPSPMQRLLQPFQPGSKPPFLATPPLPPLQSAPPPSQPPVKSKSCEFCGKT
FKFQSNLVVHRRSHTGEKPYKCNLCDHACTQASKLKRHMKTHMHKSSPMTVKSDDGLSTASSPEPGTSDLVGSAS
SALKSVVAKFKSENDPNLIPENGDEEEEEDDEEEEEEEEEEEEEEELTESERVDYGFGLSLEAARHHENSSRGAVVGVGD
ESRALPDVMQGMVLSSMQHFSEAFHQVLGEKHKRGHLAEAEGHRDTCDEDSVAGESDRIDDGTVNGRGCSPGE
SASGGLSKKLLLGSPSSLSPFSKRIKLEKEFDLPPAAMPNTENVYSQWLAGYAASRQLKDPFLSFGDSRQSPFASSSE
HSSENGSLRFSTPPGELDGGISGRSGTGSGGSTPHISGPGPGRPSSKEGRRSDTCEYCGKVFKNCSNLTVHRRSHTG
ERPYKCELCNYACAQSSKLTRHMKTHGQVGKDVYKCEICKMPFSVYSTLEKHMKKWHSDRVLNNDIKTE
Nucleic acid sequence
Human BCL11A EER forward primer
SEQ ID NO: 61
TCCAAACTCTCAAACCACAGG
Nucleic acid sequence
Human BCL11A EER reverse primer
SEQ ID NO: 62
GGCAAGTCAGTTGGGAACAC
Nucleic acid sequence
Human BCL11A EER CRISPR/Cas target sequence 1 (BCL11A genomic
antisense sequence)
SEQ ID NO: 64
GTGATAAAAGCAACTGTTAG
Nucleic acid sequence
gRNA target-complementary sequence, complementary to SEQ ID NO: 64
SEQ ID NO: 65

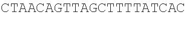

Nucleic acid sequence
Human BCL11A EER CRISPR/Cas target sequence 2 (BCL11A genomic
antisense sequence)
SEQ ID NO: 66
GGAGCCTGTGATAAAAGCAA
Nucleic acid sequence
grNA target-complementary sequence, complementary to SEQ ID NO: 66
SEQ ID NO: 67
TTGCTTTTATCACAGGCTCC
Nucleic acid sequence
Human BCL11A EER CRISPR/Cas target sequence 3 (BCL11A genomic
antisense sequence)
SEQ ID NO: 68
AACCCTTCCTGGAGCCTGTG
Nucleic acid sequence
gRNA target-complementary sequence, complementary to SEQ ID NO: 68
SEQ ID NO: 69

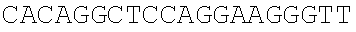

KLF1:
Protein sequence
Human Kruppel like factor 1 (″KLF1″)
SEQ ID NO: 7
MATAETALPSISTLTALGPFPDTQDDFLKWWRSEEAQDMGPGPPDPTEPPLHVKSEDQPGEEEDDERGADATW
DLDLLLTNFSGPEPGGAPQTCALAPSEAPGAQYPPPPETLGAYAGGPGLVAGLLGSEDHSGWVRPALRARAPDAF
VGPALAPAPAPEPKALALQPVYPGPGAGSSGGYFPRTGLSVPAASGAPYGLLSRYPAMYPAPQYQGHFQLFRGLQ
GPAPGPATSPSFLSCLGPGTVGTGLGGTAEDPGVIAETAPSKRGRRSWARKRQAAHTCAHPGCGKSYTKSSHLKA
HLRTHTGEKPYACTWEGCGWRFARSDELTRHYRKHTGQRPFRCQLCPRAFSRSDHLALHMKRHL
Nucleic acid sequence
Human KLF1 CRISPR/Cas target sequence 1 (KLF1 genomic antisense sequence)
SEQ ID NO: 74
CTTGCGCGCCCACGAACGTC
Nucleic acid sequence
gRNA target-complementary sequence, complementary to SEQ ID NO: 74
SEQ ID NO: 75
GACGTTCGTGGGCGCGCAAG
Nucleic acid sequence
Human KLF1 CRISPR/Cas target sequence 2 (KLF1 genomic antisense sequence)
SEQ ID NO: 76
AGCGCGCGAATCTCCAGCCG
Nucleic acid sequence
gRNA target-complementary sequence, complementary to SEQ ID NO: 76
SEQ ID NO: 77
CGGCTGGAGATTCGCGCGCT

HBG1 and HBG2:
Protein sequence
Human hemoglobin subunit gamma 1 (″A-gamma-globin″ or ″HBG1″)
SEQ ID NO: 8
MGHFTEEDKATITSLWGKVNVEDAGGETLGRLLVVYPWTQRFFDSFGNLSSASAIMGNPKVKAHGKKVLTSLGDA
IKHLDDLKGTFAQLSELHCDKLHVDPENFKLLGNVLVTVLAIHFGKEFTPEVQASWQKMVTAVASALSSRYH
Nucleic acid sequence
Human HBG1 promoter region forward primer
SEQ ID NO: 81
TCTCCCAAGGAAGTCAGCAC
Nucleic acid sequence
Human HBG1 promoter region reverse primer
SEQ ID NO: 82
CTTAGAAACCACTGCTAACTGAAAGAG
Protein sequence
Human hemoglobin subunit gamma 2 (″G-gamma-globin″ or ″HBG2″)
SEQ ID NO: 9
MGHFTEEDKATITSLWGKVNVEDAGGETLGRLLVVYPWTQRFFDSFGNLSSASAIMGNPKVKAHGKKVLTSLGDA
IKHLDDLKGTFAQLSELHCDKLHVDPENFKLLGNVLVTVLAIHFGKEFTPEVQASWQKMTVTGVASALSSRYH
Nucleic acid sequence
Human HBG2 promoter region forward primer
SEQ ID NO: 91
CAGAGGACAGGTTGCCAAAG
Nucleic acid sequence
Human HBG2 promoter region reverse primer
SEQ ID NO: 92
CCAATGCTTACTAAATGAGACTAAGACG
Nucleic acid sequence
Human HBG1 and HBG2 promoter region CRISPR/Cas target sequence 1
SEQ ID NO: 84
TGACCAATAGCCTTGACAAG
Nucleic acid sequence
gRNA target-complementary sequence, complementary to SEQ ID NO: 84
SEQ ID NO: 85

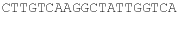

Luciferase (control):
Nucleic acid sequence
Luciferase CRISPR/Cas target sequence 1
SEQ ID NO: 54
ACCCAACGGACATTTCGAAG
Nucleic acid sequence
gRNA luciferase target-complementary sequence
SEQ ID NO: 55

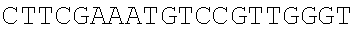

ssODN for correcting to HBB wild type:
Nucleic acid sequence
Human beta-globin (WT)-encoding ssODN sequence 1
SEQ ID NO: 101

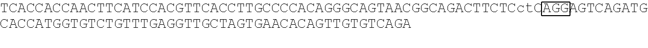

Nucleic acid sequence
Human beta-globin (WT)-encoding ssODN sequence 2
SEQ ID NO: 102

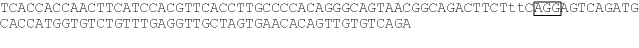

Nucleic acid sequence
Human beta-globin (WT)-encoding ssODN sequence 3
SEQ ID NO: 103

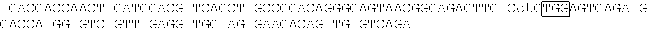

Nucleic acid sequence
Human beta-globin (WT)-encoding ssODN sequence 4
SEQ ID NO: 104

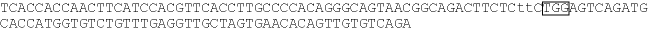

Nucleic acid sequence
Human beta-globin (WT)-encoding ssODN sequence 5
SEQ ID NO: 105

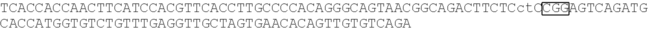

Nucleic acid sequence
Human beta-globin (WT)-encoding ssODN sequence 6
SEQ ID NO: 106

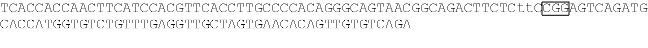

Nucleic acid sequence
Human beta-globin (WT)-encoding ssODN sequence 7
SEQ ID NO: 107

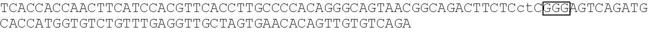

Nucleic acid sequence
Human beta-globin (WT)-encoding ssODN sequence 8
SEQ ID NO: 108

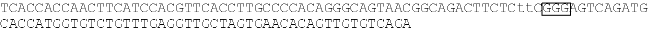

Nucleic acid sequence
Human beta-globin (WT)-encoding ssODN sequence 9 (80 bp Forward)
SEQ ID NO: 169
AGCAACCTCAAACAGACACCATGGTGCATCTGACTCCTGAGGAGAAGTCTGCCGTTACTGCCCTGTGGGGCAA
GGTGAAC
Nucleic acid sequence
Human beta-globin (WT)-encoding ssODN sequence 10 (80 bp Reverse)
SEQ ID NO: 170
GTTCACCTTGCCCCACAGGGCAGTAACGGCAGACTTCTCCTCAGGAGTCAGATGCACCATGGTGTCTGTTTGA
GGTTGCT
Nucleic acid sequence
Human beta-globin (WT)-encoding ssODN sequence 11 (100 bp Forward)
SEQ ID NO: 171
TGTGTTCACTAGCAACCTCAAACAGACACCATGGTGCATCTGACTCCTGAGGAGAAGTCTGCCGTTACTGCCCT
GTGGGGCAAGGTGAACGTGGATGAAG
Nucleic acid sequence
Human beta-globin (WT)-encoding ssODN sequence 12 (100 bp Reverse)
SEQ ID NO: 172
CTTCATCCACGTTCACCTTGCCCCACAGGGCAGTAACGGCAGACTTCTCCTCAGGAGTCAGATGCACCATGGT
GTCTGTTTGAGGTTGCTAGTGAACACA
Nucleic acid sequence
Human beta-globin (WT)-encoding ssODN sequence 13 (120 bp Forward)
SEQ ID NO: 173
CTGACACAACTGTGTTCACTAGCAACCTCAAACAGACACCATGGTGCATCTGACTCCTGAGGAGAAGTCTGCC
GTTACTGCCCTGTGGGGCAAGGTGAACGTGGATGAAGTTGGTGGTGA
Nucleic acid sequence
Human beta-globin (WT)-encoding ssODN sequence 14 (120 bp Reverse)
SEQ ID NO: 174
TCACCACCAACTTCATCCACGTTCACCTTGCCCCACAGGGCAGTAACGGCAGACTTCTCCTCAGGAGTCAGATG
CACCATGGTGTCTGTTTGAGGTTGCTAGTGAACACAGTTGTGTCAG
Nucleic acid sequence
Human beta-globin (WT)-encoding ssODN sequence 15 (with one missense; 111 bp Forward)
SEQ ID NO: 175

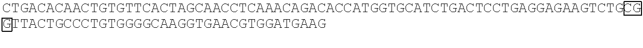

Nucleic acid sequence
Human beta-globin (WT)-encoding ssODN sequence 16 (with one missense; 111 bp Reverse)
SEQ ID NO: 176

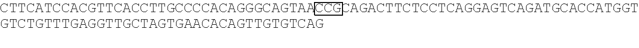

ssODN parts:
Nucleic acid sequence
5′ homology arm (60 nt) of SEQ ID NO: 101, 103, 105, and 107
SEQ ID NO: 111
TCACCACCAACTTCATCCACGTTCACCTTGCCCCACAGGGCAGTAACGGCAGACTTCTCc
Nucleic acid sequence
5′ homology (59 nt) of SEQ ID NO: 102, 104, 106, and 108
SEQ ID NO: 112
TCACCACCAACTTCATCCACGTTCACCTTGCCCCACAGGGCAGTAACGGCAGACTTCTC
Nucleic acid sequence
3′ homology arm (60 nt) of SEQ ID NO: 101, 102
SEQ ID NO: 121

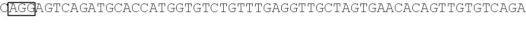

Nucleic acid sequence
3′ homology arm (58 nt) of SEQ ID NO: 103, 104, 105, 106, 107, and 108
SEQ ID NO: 122

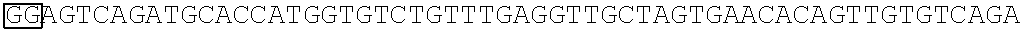

ssODN for introducing HBB E6V subtitution:
Nucleic acid sequence
Human beta-globin (E6V)-encoding ssODN sequence 1 (80 bp F)
SEQ ID NO: 181

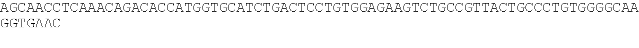

Nucleic acid sequence
Human beta-globin (E6V)-encoding ssODN sequence 2 (80 bp R)
SEQ ID NO: 182

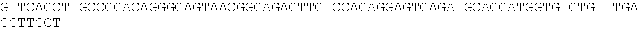

Nucleic acid sequence
Human beta-globin (E6V)-encoding ssODN sequence 3 (100 bp F)
SEQ ID NO: 183

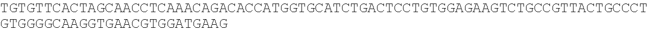

Nucleic acid sequence
Human beta-globin (E6V)-encoding ssODN sequence 4 (100 bp R)
SEQ ID NO: 184

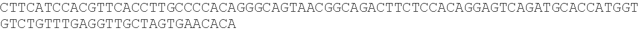

gRNA subparts:
Nucleic acid sequence
crRNA flagpole sequence (option 1)
SEQ ID NO: 131
GUUUUAGAGCUA
Nucleic acid sequence
crRNA flagpole sequence (option 2)
SEQ ID NO: 132

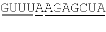

Nucleic acid sequence
crRNA first flagpole extension (optional)
SEQ ID NO: 133
UGCUG
Nucleic acid sequence
crRNA second flagpole extension (optional)
SEQ ID NO: 134
UUUUG
Nucleic acid sequence (optional)
tracrRNA first extension
SEQ ID NO: 135
CAGCA
Nucleic acid sequence
tracrRNA flagpole (option 1)
SEQ ID NO: 136
UAGCAAGUUAAAA
Nucleic acid sequence
tracrRNA flagpole (option 2)
SEQ ID NO: 137

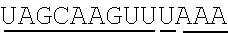

Nucleic acid sequence
tracrRNA nuclease binding domain (may be followed by multiple uracils)
SEQ ID NO: 138
UAAGGCUAGUCCGUUAUCAACUUGAAAAAGUGGCACCGAGUCGGUGC
Nucleic acid sequence
Linker (linker between crRNA sequence and tracrRNA sequence)
SEQ ID NO: 139
GAAA
Nucleic acid sequence
sgRNA backbone (3′ to targeting sequence) (option 1) (may be followed
by multiple uracils)
SEQ ID NO: 141
GUUUUAGAGCUAGAAAUAGCAAGUUAAAAUAAGGCUAGUCCGUUAUCAACUUGAAAAAGUGGCACCGA
GUCGGUGC
Nucleic acid sequence
sgRNA backbone (3′ to targeting sequence) (option 2) (may be followed
by multiple uracils)
SEQ ID NO: 142

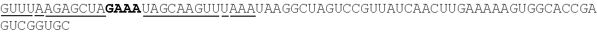

Nucleic acid sequence
sgRNA backbone (3′ to targeting sequence) (option 3) (may be followed
by multiple uracils)
SEQ ID NO: 143
GUUUUAGAGCUAUGCUGGAAACAGCAUAGCAAGUUAAAAUAAGGCUAGUCCGUUAUCAACUUGAAAAA
GUGGCACCGAGUCGGUGC
Nucleic acid sequence
sgRNA backbone (3′ to targeting sequence) (option 4) (may be followed
by multiple uracils)
SEQ ID NO: 144

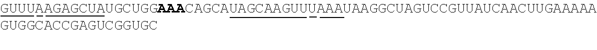

Nucleic acid sequence
dgRNA crRNA backbone (option 1)
SEQ ID NO: 145
GUUUUAGAGCUAUGCUGUUUUG
Nucleic acid sequence
dgRNA tracrRNA (option 1)
SEQ ID NO: 146

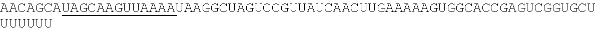

Nucleic acid sequence
dgRNA crRNA backbone (option 2)
SEQ ID NO: 147

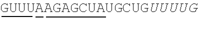

Nucleic acid sequence
dgRNA tracrRNA (option 2)
SEQ ID NO: 148

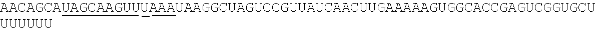

SpCas9 and Cas9 variants:
Protein sequence
SpCas9 WT
SEQ ID NO: 150
MDKKYSIGLDIGTNSVGWAVITDEYKVPSKKFKVLGNTDRHSIKKNLIGALLFDSGETAEATRLKRTARRRYTRRKNR
ICYLQEIFSNEMAKVDDSFFHRLEESFLVEEDKKHERHPIFGNIVDEVAYHEKYPTIYHLRKKLVDSTDKADLRLIYLAL
AHMIKFRGHFLIEGDLNPDNSDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAKNLSDAILLSDILRVNTEITKAP
LSASMIKRYDEHHQDLTLLKALVRQQLPEKYKEIFFDQSKNGYAGYIDGGASQEEFYKFIKPILEKMDGTEELLVKLN
REDLLRKQRTFDNGSIPHQIHLGELHAILRRQEDFYPFLKDNREKIEKILTFRIPYYVGPLARGNSRFAWMTRKSEETI
TPWNFEEVVDKGASAQSFIERMTNFDKNLPNEKVLPKHSLLYEYFTVYNELTKVKYVTEGMRKPAFLSGEQKKAIV
DLLFKTNRKVTVKQLKEDYFKKIECFDSVEISGVEDRFNASLGTYHDLLKIIKDKDFLDNEENEDILEDIVLTLTLFEDRE
MIEERLKTYAHLFDDKVMKQLKRRRYTGWGRLSRKLINGIRDKQSGKTILDFLKSDGFANRNFMQLIHDDSLTFKE
DIQKAQVSGQGDSLHEHIANLAGSPAIKKGILQTVKVVDELVKVMGRHKPENIVIEMARENQTTQKGQKNSRERM
KRIEEGIKELGSQILKEHPVENTQLQNEKLYLYYLQNGRDMYVDQELDINRLSDYDVDHIVPQSFLKDDSIDNKVLTR
SDKNRGKSDNVPSEEVVKKMKNYWRQLLNAKLITQRKFDNLTKAERGGLSELDKAGFIKRQLVETRQITKHVAQIL
DSRMNTKYDENDKLIREVKVITLKSKLVSDFRKDFQFYKVREINNYHHAHDAYLNAVVGTALIKKYPKLESEFVYGDY
KVYDVRKMIAKSEQEIGKATAKYFFYSNIMNFFKTEITLANGEIRKRPLIETNGETGEIVWDKGRDFATVRKVLSMP
QVNIVKKTEVQTGGFSKESILPKRNSDKLIARKKDWDPKKYGGFDSPTVAYSVLVVAKVEKGKSKKLKSVKELLGITI
MERSSFEKNPIDFLEAKGYKEVKKDLIIKLPKYSLFELENGRKRMLASAGELQKGNELALPSKYVNFLYLASHYEKLKG
SPEDNEQKQLFVEQHKHYLDEIIEQISEFSKRVILADANLDKVLSAYNKHRDKPIREQAENIIHLFTLTNLGAPAAFKYF
DTTIDRKRYTSTKEVLDATLIHQSITGLYETRIDLSQLGGD
Protein sequence
SpCas9 Variant 1
SEQ ID NO: 151
MAPKKKRKVDKKYSIGLDIGTNSVGWAVITDEYKVPSKKFKVLGNTDRHSIKKNLIGALLFDSGETAEATRLKRTARR
RYTRRKNRICYLQEIFSNEMAKVDDSFFHRLEESFLVEEDKKHERHPIFGNIVDEVAYHEKYPTIYHLRKKLVDSTDKA
DLRLIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSDILR
VNTEITKAPLSASMIKRYDEHHQDLTLLKALVRQQLPEKYKEIFFDQSKNGYAGYIDGGASQEEFYKFIKPILEKMDG
TEELLVKLNREDLLRKQRTFDNGSIPHQIHLGELHAILRRQEDFYPFLKDNREKIEKILTFRIPYYVGPLARGNSRFAW
MTRKSEETITPWNFEEVVDKGASAQSFIERMTNFDKNLPNEKVLPKHSLLYEYFTVYNELTKVKYVTEGMRKPAFLS
GEQKKAIVDLLFKTNRKVTVKQLKEDYFKKIECFDSVEISGVEDRFNASLGTYHDLLKIIKDKDFLDNEENEDILEDIVL
TLTLFEDREMIEERLKTYAHLFDDKVMKQLKRRRYTGWGRLSRKLINGIRDKQSGKTILDFLKSDGFANRNFMQLIH
DDSLTFKEDIQKAQVSGQGDSLHEHIANLAGSPAIKKGILQTVKVVDELVKVMGRHKPENIVIEMARENQTTQKGQ
KNSRERMKRIEEGIKELGSQILKEHPVENTQLQNEKLYLYYLQNGRDMYVDQELDINRLSDYDVDHIVPQSFLKDDS
IDNKVLTRSDKNRGKSDNVPSEEVVKKMKNYWRQLLNAKLITQRKFDNLTKAERGGLSELDKAGFIKRQLVETRQIT
KHVAQILDSRMNTKYDENDKLIREVKVITLKSKLVSDFRKDFQFYKVREINNYHHAHDAYLNAVVGTALIKKYPKLES
EFVYGDYKVYDVRKMIAKSEQEIGKATAKYFFYSNIMNFFKTEITLANGEIRKRPLIETNGETGEIVWDKGRDFATVR
KVLSMPQVNIVKKTEVQTGGFSKESILPKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRKRMLASAGELQKGNELALPSKYVNFLYLASHY
EKLKGSPEDNEQKQLFVEQHKHYLDEIIEQISEFSKRVILADANLDKVLSAYNKHRDKPIREQAENIIHLFTLTNLGAP
AAFKYFDTTIDRKRYTSTKEVLDATLIHQSITGLYETRIDLSQLGGDSRADPKKKRKVHHHHHH
Protein sequence
SpCas9 Variant 2
SEQ ID NO: 152
MAPKKKRKVDKKYSIGLDIGTNSVGWAVITDEYKVPSKKFKVLGNTDRHSIKKNLIGALLFDSGETAEATRLKRTARR
RYTRRKNRILYLQEIFSNEMAKVDDSFFHRLEESFLVEEDKKHERHPIFGNIVDEVAYHEKYPTIYHLRKKLVDSTDKA
DLRLIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSDILR
VNTEITKAPLSASMIKRYDEHHQDLTLLKALVRQQLPEKYKEIFFDQSKNGYAGYIDGGASQEEFYKFIKPILEKMDG
TEELLVKLNREDLLRKQRTFDNGSIPHQIHLGELHAILRRQEDFYPFLKDNREKIEKILTFRIPYYVGPLARGNSRFAW
MTRKSEETITPWNFEEVVDKGASAQSFIERMTNFDKNLPNEKVLPKHSLLYEYFTVYNELTKVKYVTEGMRKPAFLS
GEQKKAIVDLLFKTNRKVTVKQLKEDYFKKIEEFDSVEISGVEDRFNASLGTYHDLLKIIKDKDFLDNEENEDILEDIVLT
LTLFEDREMIEERLKTYAHLFDDKVMKQLKRRRYTGWGRLSRKLINGIRDKQSGKTILDFLKSDGFANRNFMQLIHD
DSLTFKEDIQKAQVSGQGDSLHEHIANLAGSPAIKKGILQTVKVVDELVKVMGRHKPENIVIEMARENQTTQKGQK
NSRERMKRIEEGIKELGSQILKEHPVENTQLQNEKLYLYYLQNGRDMYVDQELDINRLSDYDVDHIVPQSFLKDDSI
DNKVLTRSDKNRGKSDNVPSEEVVKKMKNYWRQLLNAKLITQRKFDNLTKAERGGLSELDKAGFIKRQLVETRQIT
KHVAQILDSRMNTKYDENDKLIREVKVITLKSKLVSDFRKDFQFYKVREINNYHHAHDAYLNAVVGTALIKKYPKLES
EFVYGDYKVYDVRKMIAKSEQEIGKATAKYFFYSNIMNFFKTEITLANGEIRKRPLIETNGETGEIVWDKGRDFATVR
KVLSMPQVNIVKKTEVQTGGFSKESILPKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRKRMLASAGELQKGNELALPSKYVNFLYLASHY
EKLKGSPEDNEQKQLFVEQHKHYLDEIIEQISEFSKRVILADANLDKVLSAYNKHRDKPIREQAENIIHLFTLTNLGAP
AAFKYFDTTIDRKRYTSTKEVLDATLIHQSITGLYETRIDLSQLGGDSRADPKKKRKVHHHHHH
Protein sequence
SpCas9 Variant 3
SEQ ID NO: 153
MGSSHHHHHHHHENLYFQGSMDKKYSIGLDIGTNSVGWAVITDEYKVPSKKFKVLGNTDRHSIKKNLIGALLFDSG
ETAEATRLKRTARRRYTRRKNRICYLQEIFSNEMAKVDDSFFHRLEESFLVEEDKKHERHPIFGNIVDEVAYHEKYPTI
YHLRKKLVDSTDKADLRLIYLALAHMIKFRGHFLIEGDLNPDNSDVDKLFIQLVQTYNQLFEENPINASGVDAKAILSA
RLSKSRRLENLIAQLPGEKKNGLFGNLIALSLGLTPNFKSNFDLAEDAKLQLSKDTYDDDLDNLLAQIGDQYADLFLA
AKNLSDAILLSDILRVNTEITKAPLSASMIKRYDEHHQDLTLLKALVRQQLPEKYKEIFFDQSKNGYAGYIDGGASQEE
FYKFIKPILEKMDGTEELLVKLNREDLLRKQRTFDNGSIPHQIHLGELHAILRRQEDFYPFLKDNREKIEKILTFRIPYYV
GPLARGNSRFAWMTRKSEETITPWNFEEVVDKGASAQSFIERMTNFDKNLPNEKVLPKHSLLYEYFTVYNELTKVK
YVTEGMRKPAFLSGEQKKAIVDLLFKTNRKVTVKQLKEDYFKKIECFDSVEISGVEDRFNASLGTYHDLLKIIKDKDFL
DNEENEDILEDIVLTLTLFEDREMIEERLKTYAHLFDDKVMKQLKRRRYTGWGRLSRKLINGIRDKQSGKTILDFLKSD
GFANRNFMQLIHDDSLTFKEDIQKAQVSGQGDSLHEHIANLAGSPAIKKGILQTVKVVDELVKVMGRHKPENIVIE
MARENQTTQKGQKNSRERMKRIEEGIKELGSQILKEHPVENTQLQNEKLYLYYLQNGRDMYVDQELDINRLSDYD
VDHIVPQSFLKDDSIDNKVLTRSDKNRGKSDNVPSEEVVKKMKNYWRQLLNAKLITQRKFDNLTKAERGGLSELDK
AGFIKRQLVETRQITKHVAQILDSRMNTKYDENDKLIREVKVITLKSKLVSDFRKDFQFYKVREINNYHHAHDAYLNA
VVGTALIKKYPKLESEFVYGDYKVYDVRKMIAKSEQEIGKATAKYFFYSNIMNFFKTEITLANGEIRKRPLIETNGETGE
IVWDKGRDFATVRKVLSMPQVNIVKKTEVQTGGFSKESILPKRNSDKLIARKKDWDPKKYGGFDSPTVAYSVLVVA
KVEKGKSKKLKSVKELLGITIMERSSFEKNPIDFLEAKGYKEVKKDLIIKLPKYSLFELENGRKRMLASAGELQKGNELA
LPSKYVNFLYLASHYEKLKGSPEDNEQKQLFVEQHKHYLDEIIEQISEFSKRVILADANLDKVLSAYNKHRDKPIREQA
ENIIHLFTLTNLGAPAAFKYFDTTIDRKRYTSTKEVLDATLIHQSITGLYETRIDLSQLGGDGGGSPKKKRKV
Protein sequence
SpCas9 Variant 4
SEQ ID NO: 154
MAHHHHHHGGSPKKKRKVDKKYSIGLDIGTNSVGWAVITDEYKVPSKKFKVLGNTDRHSIKKNLIGALLFDSGETA
EATRLKRTARRRYTRRKNRICYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFEENPINASGVDAKAILSARLS
KSRRLENLIAQLPGEKKNGLFGNLIALSLGLTPNFKSNFDLAEDAKLQLSKDTYDDDLDNLLAQIGDQYADLFLAAKN
LSDAILLSDILRVNTEITKAPLSASMIKRYDEHHQDLTLLKALVRQQLPEKYKEIFFDQSKNGYAGYIDGGASQEEFYKF
IKPILEKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISGVEDRFNASLGTYHDLLKIIKDKDFLDNEE
NEDILEDIVLTLTLFEDREMIEERLKTYAHLFDDKVMKQLKRRRYTGWGRLSRKLINGIRDKQSGKTILDFLKSDGFA
NRNFMQLIHDDSLTFKEDIQKAQVSGQGDSLHEHIANLAGSPAIKKGILQTVKVVDELVKVMGRHKPENIVIEMAR
ENQTTQKGQKNSRERMKRIEEGIKELGSQILKEHPVENTQLQNEKLYLYYLQNGRDMYVDQELDINRLSDYDVDHI
VPQSFLKDDSIDNKVLTRSDKNRGKSDNVPSEEVVKKMKNYWRQLLNAKLITQRKFDNLTKAERGGLSELDKAGFI
KRQLVETRQITKHVAQILDSRMNTKYDENDKLIREVKVITLKSKLVSDFRKDFQFYKVREINNYHHAHDAYLNAVVG
TALIKKYPKLESEFVYGDYKVYDVRKMIAKSEQEIGKATAKYFFYSNIMNFFKTEITLANGEIRKRPLIETNGETGEIVW
DKGRDFATVRKVLSMPQVNIVKKTEVQTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPKYSLFELENGRKRMLASAGELQKGNELALPS
KYVNFLYLASHYEKLKGSPEDNEQKQLFVEQHKHYLDEIIEQISEFSKRVILADANLDKVLSAYNKHRDKPIREQAENII
HLFTLTNLGAPAAFKYFDTTIDRKRYTSTKEVLDATLIHQSITGLYETRIDLSQLGGDSRADPKKKRKV
Protein sequence
SpCas9 Variant 5
SEQ ID NO: 155
MAPKKKRKVDKKYSIGLDIGTNSVGWAVITDEYKVPSKKFKVLGNTDRHSIKKNLIGALLFDSGETAEATRLKRTARR
RYTRRKNRICYLQEIFSNEMAKVDDSFFHRLEESFLVEEDKKHERHPIFGNIVDEVAYHEKYPTIYHLRKKLVDSTDKA
DLRLIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSDILR
VNTEITKAPLSASMIKRYDEHHQDLTLLKALVRQQLPEKYKEIFFDQSKNGYAGYIDGGASQEEFYKFIKPILEKMDG
TEELLVKLNREDLLRKQRTFDNGSIPHQIHLGELHAILRRQEDFYPFLKDNREKIEKILTFRIPYYVGPLARGNSRFAW
MTRKSEETITPWNFEEVVDKGASAQSFIERMTNFDKNLPNEKVLPKHSLLYEYFTVYNELTKVKYVTEGMRKPAFLS
GEQKKAIVDLLFKTNRKVTVKQLKEDYFKKIECFDSVEISGVEDRFNASLGTYHDLLKIIKDKDFLDNEENEDILEDIVL
TLTLFEDREMIEERLKTYAHLFDDKVMKQLKRRRYTGWGRLSRKLINGIRDKQSGKTILDFLKSDGFANRNFMQLIH
DDSLTFKEDIQKAQVSGQGDSLHEHIANLAGSPAIKKGILQTVKVVDELVKVMGRHKPENIVIEMARENQTTQKGQ
KNSRERMKRIEEGIKELGSQILKEHPVENTQLQNEKLYLYYLQNGRDMYVDQELDINRLSDYDVDHIVPQSFLKDDS
IDNKVLTRSDKNRGKSDNVPSEEVVKKMKNYWRQLLNAKLITQRKFDNLTKAERGGLSELDKAGFIKRQLVETRQIT
KHVAQILDSRMNTKYDENDKLIREVKVITLKSKLVSDFRKDFQFYKVREINNYHHAHDAYLNAVVGTALIKKYPKLES
EFVYGDYKVYDVRKMIAKSEQEIGKATAKYFFYSNIMNFFKTEITLANGEIRKRPLIETNGETGEIVWDKGRDFATVR
KVLSMPQVNIVKKTEVQTGGFSKESILPKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRKRMLASAGELQKGNELALPSKYVNFLYLASHY
EKLKGSPEDNEQKQLFVEQHKHYLDEIIEQISEFSKRVILADANLDKVLSAYNKHRDKPIREQAENIIHLFTLTNLGAP
AAFKYFDTTIDRKRYTSTKEVLDATLIHQSITGLYETRIDLSQLGGDSRADHHHHHH
Protein sequence
SpCas9 Variant 6
SEQ ID NO: 156
MAHHHHHHGGSDKKYSIGLDIGTNSVGWAVITDEYKVPSKKFKVLGNTDRHSIKKNLIGALLFDSGETAEATRLKRT
ARRRYTRRKNRICYLQEIFSNEMAKVDDSFFHRLEESFLVEEDKKHERHPIFGNIVDEVAYHEKYPTIYHLRKKLVDST
DKADLRLIYLALAHMIKFRGHFLIEGDLNPDNSDVDKLFIQLVQTYNQLFEENPINASGVDAKAILSARLSKSRRLENL
IAQLPGEKKNGLFGNLIALSLGLTPNFKSNFDLAEDAKLQLSKDTYDDDLDNLLAQIGDQYADLFLAAKNLSDAILLS
DILRVNTEITKAPLSASMIKRYDEHHQDLTLLKALVRQQLPEKYKEIFFDQSKNGYAGYIDGGASQEEFYKFIKPILEK
MDGTEELLVKLNREDLLRKQRTFDNGSIPHQIHLGELHAILRRQEDFYPFLKDNREKIEKILTFRIPYYVGPLARGNSR
FAWMTRKSEETITPWNFEEVVDKGASAQSFIERMTNFDKNLPNEKVLPKHSLLYEYFTVYNELTKVKYVTEGMRKP
AFLSGEQKKAIVDLLFKTNRKVTVKQLKEDYFKKIECFDSVEISGVEDRFNASLGTYHDLLKIIKDKDFLDNEENEDILE
DIVLTLTLFEDREMIEERLKTYAHLFDDKVMKQLKRRRYTGWGRLSRKLINGIRDKQSGKTILDFLKSDGFANRNFM
QLIHDDSLTFKEDIQKAQVSGQGDSLHEHIANLAGSPAIKKGILQTVKVVDELVKVMGRHKPENIVIEMARENQTT
QKGQKNSRERMKRIEEGIKELGSQILKEHPVENTQLQNEKLYLYYLQNGRDMYVDQELDINRLSDYDVDHIVPQSF
LKDDSIDNKVLTRSDKNRGKSDNVPSEEVVKKMKNYWRQLLNAKLITQRKFDNLTKAERGGLSELDKAGFIKRQLV
ETRQITKHVAQILDSRMNTKYDENDKLIREVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEITLANGEIRKRPLIETNGETGEIVWDKGRD
FATVRKVLSMPQVNIVKKTEVQTGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRKRMLASAGELQKGNELALPSKYVNFLY
LASHYEKLKGSPEDNEQKQLFVEQHKHYLDEIIEQISEFSKRVILADANLDKVLSAYNKHRDKPIREQAENIIHLFTLTN
LGAPAAFKYFDTTIDRKRYTSTKEVLDATLIHQSITGLYETRIDLSQLGGDSRADPKKKRKV
Protein sequence
SpCas9 Variant 7
SEQ ID NO: 157
MGSSHHHHHHHHENLYFQGSMDKKYSIGLDIGTNSVGWAVITDEYKVPSKKFKVLGNTDRHSIKKNLIGALLFDSG
ETAEATRLKRTARRRYTRRKNRICYLQEIFSNEMAKVDDSFFHRLEESFLVEEDKKHERHPIFGNIVDEVAYHEKYPTI
YHLRKKLVDSTDKADLRLIYLALAHMIKFRGHFLIEGDLNPDNSDVDKLFIQLVQTYNQLFEENPINASGVDAKAILSA
RLSKSRRLENLIAQLPGEKKNGLFGNLIALSLGLTPNFKSNFDLAEDAKLQLSKDTYDDDLDNLLAQIGDQYADLFLA
AKNLSDAILLSDILRVNTEITKAPLSASMIKRYDEHHQDLTLLKALVRQQLPEKYKEIFFDQSKNGYAGYIDGGASQEE
FYKFIKPILEKMDGTEELLVKLNREDLLRKQRTFDNGSIPHQIHLGELHAILRRQEDFYPFLKDNREKIEKILTFRIPYYV
GPLARGNSRFAWMTRKSEETITPWNFEEVVDKGASAQSFIERMTNFDKNLPNEKVLPKHSLLYEYFTVYNELTKVK
YVTEGMRKPAFLSGEQKKAIVDLLFKTNRKVTVKQLKEDYFKKIECFDSVEISGVEDRFNASLGTYHDLLKIIKDKDFL
DNEENEDILEDIVLTLTLFEDREMIEERLKTYAHLFDDKVMKQLKRRRYTGWGRLSRKLINGIRDKQSGKTILDFLKSD
GFANRNFMQLIHDDSLTFKEDIQKAQVSGQGDSLHEHIANLAGSPAIKKGILQTVKVVDELVKVMGRHKPENIVIE
MARENQTTQKGQKNSRERMKRIEEGIKELGSQILKEHPVENTQLQNEKLYLYYLQNGRDMYVDQELDINRLSDYD
VDHIVPQSFLKDDSIDNKVLTRSDKNRGKSDNVPSEEVVKKMKNYWRQLLNAKLITQRKFDNLTKAERGGLSELDK
AGFIKRQLVETRQITKHVAQILDSRMNTKYDENDKLIREVKVITLKSKLVSDFRKDFQFYKVREINNYHHAHDAYLNA
VVGTALIKKYPKLESEFVYGDYKVYDVRKMIAKSEQEIGKATAKYFFYSNIMNFFKTEITLANGEIRKRPLIETNGETGE
IVWDKGRDFATVRKVLSMPQVNIVKKTEVQTGGFSKESILPKRNSDKLIARKKDWDPKKYGGFDSPTVAYSVLVVA
KVEKGKSKKLKSVKELLGITIMERSSFEKNPIDFLEAKGYKEVKKDLIIKLPKYSLFELENGRKRMLASAGELQKGNELA
LPSKYVNFLYLASHYEKLKGSPEDNEQKQLFVEQHKHYLDEIIEQISEFSKRVILADANLDKVLSAYNKHRDKPIREQA
ENIIHLFTLTNLGAPAAFKYFDTTIDRKRYTSTKEVLDATLIHQSITGLYETRIDLSQLGGDGGGSPKKKRKV
Protein sequence
SpCas9 Variant 8
SEQ ID NO: 158
MAPKKKRKVDKKYSIGLDIGTNSVGWAVITDEYKVPSKKFKVLGNTDRHSIKKNLIGALLFDSGETAEATRLKRTARR
RYTRRKNRICYLQEIFSNEMAKVDDSFFHRLEESFLVEEDKKHERHPIFGNIVDEVAYHEKYPTIYHLRKKLVDSTDKA
DLRLIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSDILR
VNTEITKAPLSASMIKRYDEHHQDLTLLKALVRQQLPEKYKEIFFDQSKNGYAGYIDGGASQEEFYKFIKPILEKMDG
TEELLVKLNREDLLRKQRTFDNGSIPHQIHLGELHAILRRQEDFYPFLKDNREKIEKILTFRIPYYVGPLARGNSRFAW
MTRKSEETITPWNFEEVVDKGASAQSFIERMTNFDKNLPNEKVLPKHSLLYEYFTVYNELTKVKYVTEGMRKPAFLS
GEQKKAIVDLLFKTNRKVTVKQLKEDYFKKIECFDSVEISGVEDRFNASLGTYHDLLKIIKDKDFLDNEENEDILEDIVL
TLTLFEDREMIEERLKTYAHLFDDKVMKQLKRRRYTGWGRLSRKLINGIRDKQSGKTILDFLKSDGFANRNFMQLIH
DDSLTFKEDIQKAQVSGQGDSLHEHIANLAGSPAIKKGILQTVKVVDELVKVMGRHKPENIVIEMARENQTTQKGQ
KNSRERMKRIEEGIKELGSQILKEHPVENTQLQNEKLYLYYLQNGRDMYVDQELDINRLSDYDVDHIVPQSFLKDDS
IDNAVLTRSDKNRGKSDNVPSEEVVKKMKNYWRQLLNAKLITQRKFDNLTKAERGGLSELDKAGFIKRQLVETRQI
TKHVAQILDSRMNTKYDENDKLIREVKVITLKSKLVSDFRKDFQFYKVREINNYHHAHDAYLNAVVGTALIKKYPKLE
SEFVYGDYKVYDVRKMIAKSEQEIGKATAKYFFYSNIMNFFKTEITLANGEIRKRPLIETNGETGEIVWDKGRDFATV
RKVLSMPQVNIVKKTEVQTGGFSKESILPKRNSDKLIARKKDWDPKKYGGFDSPTVAYSVLVVAKVEKGKSKKLKSV
KELLGITIMERSSFEKNPIDFLEAKGYKEVKKDLIIKLPKYSLFELENGRKRMLASAGELQKGNELALPSKYVNFLYLAS
HYEKLKGSPEDNEQKQLFVEQHKHYLDEIIEQISEFSKRVILADANLDKVLSAYNKHRDKPIREQAENIIHLFTLTNLG
APAAFKYFDTTIDRKRYTSTKEVLDATLIHQSITGLYETRIDLSQLGGDSRADPKKKRKVHHHHHH
Protein sequence
SpCas9 Variant 9
SEQ ID NO: 159
MAPKKKRKVDKKYSIGLDIGTNSVGWAVITDEYKVPSKKFKVLGNTDRHSIKKNLIGALLFDSGETAEATRLKRTARR
RYTRRKNRICYLQEIFSNEMAKVDDSFFHRLEESFLVEEDKKHERHPIFGNIVDEVAYHEKYPTIYHLRKKLVDSTDKA
DLRLIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSDILR
VNTEITKAPLSASMIKRYDEHHQDLTLLKALVRQQLPEKYKEIFFDQSKNGYAGYIDGGASQEEFYKFIKPILEKMDG
TEELLVKLNREDLLRKQRTFDNGSIPHQIHLGELHAILRRQEDFYPFLKDNREKIEKILTFRIPYYVGPLARGNSRFAW
MTRKSEETITPWNFEEVVDKGASAQSFIERMTNFDKNLPNEKVLPKHSLLYEYFTVYNELTKVKYVTEGMRKPAFLS
GEQKKAIVDLLFKTNRKVTVKQLKEDYFKKIECFDSVEISGVEDRFNASLGTYHDLLKIIKDKDFLDNEENEDILEDIVL
TLTLFEDREMIEERLKTYAHLFDDKVMKQLKRRRYTGWGRLSRKLINGIRDKQSGKTILDFLKSDGFANRNFMQLIH
DDSLTFKEDIQKAQVSGQGDSLHEHIANLAGSPAIKKGILQTVKVVDELVKVMGRHKPENIVIEMARENQTTQKGQ
KNSRERMKRIEEGIKELGSQILKEHPVENTQLQNEALYLYYLQNGRDMYVDQELDINRLSDYDVDHIVPQSFLKDDS
IDNKVLTRSDKNRGKSDNVPSEEVVKKMKNYWRQLLNAKLITQRKFDNLTKAERGGLSELDKAGFIKRQLVETRQIT
KHVAQILDSRMNTKYDENDKLIREVKVITLKSKLVSDFRKDFQFYKVREINNYHHAHDAYLNAVVGTALIKKYPALES
EFVYGDYKVYDVRKMIAKSEQEIGKATAKYFFYSNIMNFFKTEITLANGEIRKAPLIETNGETGEIVWDKGRDFATVR
KVLSMPQVNIVKKTEVQTGGFSKESILPKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRKRMLASAGELQKGNELALPSKYVNFLYLASHY
EKLKGSPEDNEQKQLFVEQHKHYLDEIIEQISEFSKRVILADANLDKVLSAYNKHRDKPIREQAENIIHLFTLTNLGAP
AAFKYFDTTIDRKRYTSTKEVLDATLIHQSITGLYETRIDLSQLGGDSRADPKKKRKVHHHHHH
Protein sequence
SpCas9 Variant 10
SEQ ID NO: 160
MAPKKKRKVDKKYSIGLDIGTNSVGWAVITDEYKVPSKKFKVLGNTDRHSIKKNLIGALLFDSGETAEATRLKRTARR
RYTRRKNRICYLQEIFSNEMAKVDDSFFHRLEESFLVEEDKKHERHPIFGNIVDEVAYHEKYPTIYHLRKKLVDSTDKA
DLRLIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSDILR
VNTEITKAPLSASMIKRYDEHHQDLTLLKALVRQQLPEKYKEIFFDQSKNGYAGYIDGGASQEEFYKFIKPILEKMDG
TEELLVKLNREDLLRKQRTFDNGSIPHQIHLGELHAILRRQEDFYPFLKDNREKIEKILTFRIPYYVGPLARGNSRFAW
MTRKSEETITPWNFEEVVDKGASAQSFIERMTNFDKNLPNEKVLPKHSLLYEYFTVYNELTKVKYVTEGMRKPAFLS
GEQKKAIVDLLFKTNRKVTVKQLKEDYFKKIECFDSVEISGVEDRFNASLGTYHDLLKIIKDKDFLDNEENEDILEDIVL
TLTLFEDREMIEERLKTYAHLFDDKVMKQLKRRRYTGWGRLSRKLINGIRDKQSGKTILDFLKSDGFANRNFMQLIH
DDSLTFKEDIQKAQVSGQGDSLHEHIANLAGSPAIKKGILQTVKVVDELVKVMGRHKPENIVIEMARENQTTQKGQ
KNSRERMKRIEEGIKELGSQILKEHPVENTQLQNEKLYLYYLQNGRDMYVDQELDINRLSDYDVDHIVPQSFLADDS
IDNKVLTRSDKNRGKSDNVPSEEVVKKMKNYWRQLLNAKLITQRKFDNLTKAERGGLSELDKAGFIKRQLVETRQIT
KHVAQILDSRMNTKYDENDKLIREVKVITLKSKLVSDFRKDFQFYKVREINNYHHAHDAYLNAVVGTALIKKYPALES
EFVYGDYKVYDVRKMIAKSEQEIGKATAKYFFYSNIMNFFKTEITLANGEIRKAPLIETNGETGEIVWDKGRDFATVR
KVLSMPQVNIVKKTEVQTGGFSKESILPKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRKRMLASAGELQKGNELALPSKYVNFLYLASHY
EKLKGSPEDNEQKQLFVEQHKHYLDEIIEQISEFSKRVILADANLDKVLSAYNKHRDKPIREQAENIIHLFTLTNLGAP
AAFKYFDTTIDRKRYTSTKEVLDATLIHQSITGLYETRIDLSQLGGDSRADPKKKRKVHHHHHH
Protein sequence
SpCas9 Variant 11
SEQ ID NO: 161
MAPKKKRKVDKKYSIGLDIGTNSVGWAVITDEYKVPSKKFKVLGNTDRHSIKKNLIGALLFDSGETAEATRLKRTARR
RYTRRKNRICYLQEIFSNEMAKVDDSFFHRLEESFLVEEDKKHERHPIFGNIVDEVAYHEKYPTIYHLRKKLVDSTDKA
DLRLIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSDILR
VNTEITKAPLSASMIKRYDEHHQDLTLLKALVRQQLPEKYKEIFFDQSKNGYAGYIDGGASQEEFYKFIKPILEKMDG
TEELLVKLNREDLLRKQRTFDNGSIPHQIHLGELHAILRRQEDFYPFLKDNREKIEKILTFRIPYYVGPLARGNSRFAW
MTRKSEETITPWNFEEVVDKGASAQSFIERMTAFDKNLPNEKVLPKHSLLYEYFTVYNELTKVKYVTEGMRKPAFLS
GEQKKAIVDLLFKTNRKVTVKQLKEDYFKKIECFDSVEISGVEDRFNASLGTYHDLLKIIKDKDFLDNEENEDILEDIVL
TLTLFEDREMIEERLKTYAHLFDDKVMKQLKRRRYTGWGALSRKLINGIRDKQSGKTILDFLKSDGFANRNFMALIH
DDSLTFKEDIQKAQVSGQGDSLHEHIANLAGSPAIKKGILQTVKVVDELVKVMGRHKPENIVIEMARENQTTQKGQ
KNSRERMKRIEEGIKELGSQILKEHPVENTQLQNEKLYLYYLQNGRDMYVDQELDINRLSDYDVDHIVPQSFLKDDS
IDNKVLTRSDKNRGKSDNVPSEEVVKKMKNYWRQLLNAKLITQRKFDNLTKAERGGLSELDKAGFIKRQLVETRAIT
KHVAQILDSRMNTKYDENDKLIREVKVITLKSKLVSDFRKDFQFYKVREINNYHHAHDAYLNAVVGTALIKKYPKLES
EFVYGDYKVYDVRKMIAKSEQEIGKATAKYFFYSNIMNFFKTEITLANGEIRKRPLIETNGETGEIVWDKGRDFATVR
KVLSMPQVNIVKKTEVQTGGFSKESILPKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRKRMLASAGELQKGNELALPSKYVNFLYLASHY
EKLKGSPEDNEQKQLFVEQHKHYLDEIIEQISEFSKRVILADANLDKVLSAYNKHRDKPIREQAENIIHLFTLTNLGAP
AAFKYFDTTIDRKRYTSTKEVLDATLIHQSITGLYETRIDLSQLGGDSRADPKKKRKVHHHHHH

What is claimed is:

1. One or more isolated guide RNAs (gRNAs) for Clustered Regularly Interspaced Short Palindromic Repeats (CRISPR)-mediated gene editing, wherein the gRNAs comprise at least one CRISPR RNA (crRNA) sequence comprising a target-complementary sequence comprising at least 17 nucleic acids, optionally comprising 17, 18, 19, 20, 21, 22, 23, 24, 25, 26, 27, 28, 29, or 30 nucleic acids, the target-complementary sequence comprising:
(i) the polynucleotide sequence of SEQ ID NO: 85, 25, 45, 47, 49, 65, 67, 69, 75, or 77; or
(ii) a polynucleotide sequence comprising one or more (optionally one, two, three, four, or five) mutations relative to the polynucleotide sequence of SEQ ID NO: 85, 25, 45, 47, 49, 65, 67, 69, 75, or 77, said mutations optionally at any nucleic acid position(s) other than the 4th to the 7th nucleic acid positions from the 3′-end of the polynucleotide sequence of SEQ ID NO: 85, 25, 45, 47, 49, 65, 67, 69, 75, or 77, respectively,
optionally wherein the gRNA is:
(I) a single guide RNA (sgRNA) comprising (i) a crRNA sequence comprising the target-complementary sequence and a crRNA backbone sequence and (ii) a trans-activating CRISPR RNA (tracrRNA) sequence in a single strand, optionally wherein the crRNA sequence and the tracrRNA sequence are linked via a linker optionally comprising SEQ ID NO: 139, further optionally wherein the gRNA comprises the target-complementary sequence followed by a sgRNA backbone sequence of any of SEQ ID NOS: 141-144, optionally wherein the sgRNA backbone sequence is followed by one or more uracils, further optionally 1-10 uracils, or
(II) a dual guide RNA (dgRNA) formed by hybridization between (i) a crRNA sequence comprising the target-complementary sequence and a crRNA backbone sequence and (ii) a tracrRNA, optionally wherein the crRNA backbone sequence and the tracrRNA comprise SEQ ID NOS: 145 and 146, respectively, or SEQ ID NOS: 147 and 148, respectively,
and optionally wherein:
(i) the one or more gRNAs are synthetic or recombinant; and/or
(ii) the one or more gRNAs comprise a synthetic sgRNA and comprises at least one chemical modification, optionally (ii-1) 2-O-methylation further optionally at first three and last three bases and/or (ii-2) one or more 3′ phosphorothioate bonds, further optionally between first three and last two bases.

2. A composition comprising one or more isolated gRNAs according to claim 1.

3. A polynucleotide or polynucleotides encoding the one or more isolated gRNAs of claim 1.

4. A vector comprising the polynucleotide or polynucleotides of claim 3 operably linked to one or more regulatory sequences.

5. A ribonucleoprotein (RNP), which comprises:
(a) one or more isolated gRNAs of claim 1; which is complexed with
(b) a Cas endonuclease,
optionally wherein: the Cas endonuclease is:
(i) selected from the group consisting of Cas9, Cas3, Cas8a2, Cas8b, Cas8c, Cas10, Cas11, Cas12, Cas12a or Cpf1, Cas13, Cas13a, C2c1, C2c3, and C2c2;
(ii) a class 2 Cas endonuclease, optionally a type II, type V, or type VI Cas nuclease;
(iii) Cas9 of Streptococcus pyogenes (SpCas9), Staphylococcus aureus Cas9 (SaCas9), Streptococcus thermophilus (StCas9), Neisseria meningitidis (NmCas9), Francisella novicida (FnCas9), Campylobacter jejuni (CjCas9), Streptococcus canis (ScCas9), Staphylococcus auricularis (SauriCas9), or any engineered variants thereof, including SaCas9-HF, SpCas9-HF1, KKHSaCas9, eSpCas9, HypaCas9, FokI-Fused dCas9, xCas9, SpRY (variant of SpCas9), and SpG (variant of SpCas9); and/or
(iv) Cas9, optionally comprising any one of SEQ ID NOS: 150-161,
and optionally wherein the RNP is formed by mixing at an approximately equimolar ratio (I) a solution comprising the one or more isolated gRNAs, optionally wherein the pH of the solution is about 6 to 8, about 6.5 to 7.5, further optionally about 7, and (II) a solution comprising the Cas endonuclease, optionally wherein the pH of the solution is about 6 to 8, about 6.5 to 7.5, further optionally about 7, further optionally wherein the mixing is for about 5 minutes.

6. A pharmaceutical composition comprising at least one cargo encapsulated in a carrier, optionally a lipid-based, transfection competent vesicle (TCV),
wherein the at least one cargo is capable of:
(i) effecting gene editing of at least one Sickle cell disease (SCD)-associated gene and/or a promoter or enhancer thereof in vivo in a subject in need thereof; and/or
(ii) altering the expression, function, and/or effect of at least one SCD-associated gene in vivo in a subject in need thereof,
optionally wherein the subject has or has a risk of developing SCD, which is optionally sickle cell anemia (SCA), Sickle cell-hemoglobin C (HbSC), or HbS β-thalassaemia,
optionally wherein the composition is for:
(I) direct injection into the bone marrow of the subject; and/or
(II) intravenous injection into the subject, optionally wherein the subject is administered at least one agent that promotes stem cell mobilization.

7. The pharmaceutical composition of claim 6, wherein the carrier is a lipid-based TCV which comprises at least one ionizable cationic lipid, optionally wherein:
(i) the at least one ionizable cationic lipid comprises, essentially consists of, or consists of a lipid selected from the group consisting of N,N-dimethyl-2,3-dioleyloxy)propylamine (DODMA), 1,2-dioleoyl-3-dimethylammonium propane (“DODAP”), 1,2-Dilinoleoyl-3-dimethylaminopropane (DLinDAP), N,N-dimethyl-2,2-di-(9Z,12Z)-9,12-octadecadien-1-yl-1,3-dioxolane-4-ethanamine (KC2), (6Z,9Z,28Z,31Z)-heptatriaconta-6,9,28,31-tetraen-19-yl 4-(dimethylamino)butanoate (MC3), N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxyl)propyl)-N,N,N-trimethylammonium chloride (DOTMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA·Cl), 1,2-Dilinoleoyl-3-trimethylaminopropane chloride salt (DLin-TAR·Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLin-K-DMA), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyl)tetrahydro-3 aH-cyclopenta[d][1,3]dioxol-5-amine (ALNY-100), N-(2,3-dioleyloxyl)propyl-N,N—N-triethylammonium chloride (“DOTMA”); 1,2-Dioleyloxy-3-trimethylaminopropane chloride salt (“DOTAP·Cl”); 3.beta.-(N—(N′,N′-dimethylaminoethane)-carbamoyl)cholesterol (“DC-Chol”), N-(1-(2,3-dioleyloxyl)propyl)-N-2-(sperminecarboxamido)ethyl)-N,N-dimethyl-ammonium trifluoracetate (“DOSPA”), dioctadecylamidoglycyl carboxyspermine (“DOGS”), and N-(1,2-dimyristyloxyprop-3-yl)-N,N-dimethyl-N-hydroxyethyl ammonium bromide (“DMRIE”), and any combinations thereof;
(ii) the TCV further comprises at least one helper lipid, optionally wherein the at least one helper lipid comprises, essentially consists of, or consists of a lipid selected from the group consisting of dioleoylphosphatidylethanolamine (DOPE), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), and any combinations thereof;
(iii) the TCV further comprises at least one phospholipid, optionally wherein the at least one phospholipid comprises, essentially consists of, or consists of a lipid selected from the group consisting of distearoylphosphatidylcholine (DSPC), dioleoyl phosphatidylethanolamine (DOPE), dipalmitoylphosphatidylcholine (DPPC), phosphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dilinoleoylphosphatidylcholine distearoylphophatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine, and any combinations thereof;
(iv) the TCV further comprises at least one cholesterol or cholesterol derivative, optionally wherein the at least one cholesterol or cholesterol derivative comprises, essentially consists of, or consists of a cholesterol or cholesterol derivative selected from the group consisting of cholesterol, N,N-dimethyl-N-ethylcarboxamidocholesterol (DC-Chol), 1,4-bis(3-N-oleylamino-propyl)piperazine, imidazole cholesterol ester (ICE), and any combinations thereof;
(v) the TCV further comprises at least one PEG or PEG-lipid, optionally wherein the at least one PEG-lipid comprises, essentially consists of, or consists of a PEG-lipid selected from the group consisting of PEG-myristoyl diglyceride (PEG-DMG) (e.g., 1,2-dimyristoyl-rac-glycero-3-methoxypolyethylene glycol-2000 (Avanti® Polar Lipids (Birmingham, AL)), which is a mixture of 1,2-DMG PEG2000 and 1,3-DMG PEG2000 (e.g., in about 97:3 ratio)), PEG-phosphatidylethanolamine and phosphatidic acid, PEG-ceramide conjugates (e.g., PEG-CerC14 or PEG-CerC20), PEG-modified dialkylamines, PEG-modified 1,2-diacyloxypropan-3-amines, and any combinations thereof; and/or
(vi) the TCV is substantially, essentially, or entirely free of destabilizing agents,
or any combination of (i) to (v),
optionally wherein the TCV is formed by:
(a) generating a first solution by dissolving all components of the TCV, optionally at about 20-35 mM, in ethanol;
(b) providing a second solution, which is aqueous and contains sodium acetate and/or sodium citrate, optionally at about 25 mM, optionally wherein the pH of the solution is about 4;
(c) combining the first and second solutions by gentle mixing (optionally repeated manual reciprocation of the TCV-generating fluid in a pipette), micromixing optionally using a staggered herringbone micromixer (SHM) or T-junction or Y-junction mixing, or extrusion; and
(d) removing ethanol, optionally by dialysis or evaporation,
further optionally the size of the TCV before encapsulation of the at least one cargo is in a range of about 9 nm to about 80 nm at pH of about 4.

8. The pharmaceutical composition of claim 6 or 7, wherein:
(a) the amount of the at least one ionizable cationic lipid relative to the total components of the TCV is: (a-1) about 10 mol % to about 70 mol %, about 10 mol % to about 60 mol %, about 10 mol % to about 50 mol %, about 10 mol % to about 40 mol %, about 10 mol % to about 30 mol %, about 15 mol % to about 25 mol %, about 18 mol % to about 22 mol %, about 19 mol % to about 21 mol %, about 19.5 mol % to about 20.5 mol %, about 19.8 mol % to about 20.2 mol %, or about 20 mol %; or (a-2) about 10 mol % to about 70 mol %, about 20 mol % to about 70 mol %, about 30 mol % to about 70 mol %, about 40 mol % to about 70 mol %, about 40 mol % to about 60 mol %, about 45 mol % to about 55 mol %, about 48 mol % to about 52 mol %, about 49 mol % to about 51 mol %, about 49.5 mol % to about 50.5 mol %, about 49.8 mol % to about 50.2 mol %, or about 50 mol %;
(b) in (ii), the amount of the at least one helper lipid relative to the total components of the TCV is about 10 mol % to about 60 mol %, about 10 mol % to about 50 mol %, about 10 mol % to about 40 mol %, about 20 mol % to about 40 mol %, about 25 mol % to about 35 mol %, about 28 mol % to about 32 mol %, about 29 mol % to about 31 mol %, about 29.5 mol % to about 30.5 mol %, about 29.8 mol % to about 30.2 mol %, or about 30 mol %;
(c) in (iii), the amount of the at least one phospholipid relative to the total components of the TCV is about 5 mol % to about 65 mol %, about 5 mol % to about 55 mol %, about 5 mol % to about 45 mol %, about 5 mol % to about 35 mol %, about 5 mol % to about 25 mol %, about 5 mol % to about 15 mol %, about 8 mol % to about 12 mol %, about 9 mol % to about 11 mol %, about 9.5 mol % to about 10.5 mol %, about 9.8 mol % to about 10.2 mol %, or about 10 mol %;
(d) in (iv), the amount of the at least one cholesterol or cholesterol derivative relative to the total components of the TCV is about 20 mol % to about 60 mol %, about 25 mol % to about 55 mol %, about 30 mol % to about 50 mol %, about 35 mol % to about 45 mol %, about 38 mol % to about 42 mol %, about 39 mol % to about 41 mol %, about 39.5 mol % to about 40.5 mol %, about 39.8 mol % to about 40.2 mol %, or about 40 mol %, or about 39%; and/or
(e) in (v), the amount of the at least one PEG or PEG-lipid relative to the total components of the TCV is about 0.1 mol % to about 5 mol %, 0.1 mol % to about 4 mol %, 0.1 mol % to about 3 mol %, 0.1 mol % to about 2 mol %, 0.5 mol % to about 1.5 mol %, 0.8 mol % to about 1.2 mol %, 0.9 mol % to about 1.1 mol %, or about 1 mol %.

9. The pharmaceutical composition of any one of claims 6-8, wherein:
(I) the TCV comprises, essentially consists of, or consists of:
(i) at least one ionizable cationic lipid, which is optionally DODMA;
(ii) at least one helper lipid, which is optionally DOPE;
(iii) at least one phospholipid, which is optionally DSPC; and
(iv) at least one cholesterol or cholesterol derivative,
optionally wherein the amounts of the at least one ionizable cationic lipid, the at least one helper lipid, the at least one phospholipid, and the at least one cholesterol or cholesterol derivative, relative to the total components of the TCV, is about 20 mol %, about 30 mol %, about 10 mol %, and about 40 mol %, respectively; or
(II) the TCV comprises, essentially consists of, or consists of:
(i) at least one ionizable cationic lipid, which is optionally DODMA;
(ii) at least one helper lipid, which is optionally DOPE;
(iii) at least one phospholipid, which is optionally DSPC;
(iv) at least one cholesterol or cholesterol derivative; and
(v) at least one PEG or PEG-lipid, which is optionally PEG-DMG,
optionally wherein the amounts of the at least one ionizable cationic lipid, the at least one helper lipid, the at least one phospholipid, the at least one cholesterol or cholesterol derivative, and the at least one PEG or PEG-lipid, relative to the total components of the TCV, is about 20 mol %, about 30 mol %, about 10 mol %, about 39 mol %, and about 1 mol %, respectively,
and wherein the TCV is substantially, essentially, or entirely free of ethanol, methanol, isopropanol, tetrahydrofuran (THF), dimethyl sulfoxide (DMSO), dimethyl formamide (DMF), and acetonitrile (ACN),
optionally wherein the TCV is substantially, essentially, or entirely free of organic solvents and detergents,
further optionally wherein the TCV is substantially, essentially, or entirely free of destabilizing agents and/or is stable for prolonged periods of time at about 1 to about 40° C., about 5 to about 35° C., about 10 to about 30° C., or about 15 to about 25° C.,
and further optionally wherein the TCV or the pharmaceutical composition further comprises and/or is stored in the presence of at least one cryoprotectant, optionally wherein:
(a) the cryoprotectant comprises a sugar-based molecule, which is optionally sucrose, trehalose, or a combination thereof;
(b) the concentration of the cryoprotectant is about 1% to about 40%, about 3% to about 30%, about 5% to about 30%, about 10% to about 20%, or about 15%;
(c) the TCV is stable at a freezing temperature, optionally at about −20° C. or about −80° C., optionally for at least about one week, at least about two weeks, at least about three weeks, at least about a month, at least about two months, at least about four months, at least about five months, at least about six months, at least about nine months, at least about a year, or at least about two years, or longer, further optionally for about one week to about two year, about two weeks to about a year, about three weeks to about nine month, about one to about six months, about one to five months, about one to four months, about one to three months, or about one to two months; or
(d) any combination of (a)-(c).

10. The pharmaceutical composition of any one of claims 6-9, wherein the at least one SCD-associated gene:
(i) comprises one or more genes selected from the group consisting of HBB (the sickle cell hemoglobin (HbS) variant, also known as the βS allele), BCL11A, KLF1, SOX6, GATA1, NF-E4 (or NFE4), COUP-TF, NR2C1 (also known as TR2), NR2C2 (also known as TR4), genes encoding members of the MBD2 protein complex, IKZF1 (also known as Ikaros), genes encoding other members of PYR complex (CHD4, HDAC2, RBBP7, SMARCB1, SMARCC1, SM4RCC2, SMARCD1, and SMARCE1), BRG1, and genes that directly or indirectly modulate the expression thereof;
(ii) is HBB (such as the sickle cell hemoglobin (HbS) variant of HBB, also known as the βS allele, or the hemoglobin C (HbC) variant of HBB), optionally comprising the polynucleotide sequence of SEQ ID NO: 11, 21 or 31 and/or encoding the amino acid sequence of SEQ ID NO: 1, 2 or 3, and/or a promoter or enhancer region of HBB;
(iii) is BCL11A, optionally encoding the amino acid sequence of SEQ ID NO: 6, and/or a promoter or enhancer region of BCL11A, preferably the erythroid-enhancer region (EER) of BCL11A;
(iv) is KLF1, optionally encoding the amino acid sequence of SEQ ID NO: 7, and/or a promoter or enhancer region of KLF1;
(v) is HBG1, optionally encoding the amino acid sequence of SEQ ID NO: 8, and/or a promoter or enhancer region of HBG1; and/or
(vi) is HBG2, optionally encoding the amino acid sequence of SEQ ID NO: 9, and/or a promoter or enhancer region of HBG2.

11. The pharmaceutical composition of any one of claims 6-10, wherein:
(I) the gene editing is mediated by a protease, nuclease, endonuclease, meganuclease, zinc finger nuclease (ZFN), transcription activator-like nuclease (TALEN), or clustered regularly interspaced short palindromic repeats (CRISPR)-associated (Cas) nuclease, optionally resulting in at least one nucleic acid insertion, deletion, or replacement (e.g., resulting in a nonsense, missense, or silent mutation) in the at least one SCD-associated gene; and/or
(II) the at least one cargo is capable of effecting gene editing comprises, essentially consists of, or consists of:
(a) a Cas nuclease, a RNA encoding a Cas nuclease, or a nucleic acid such as a DNA or RNA encoding a Cas nuclease, optionally wherein the Cas nuclease is:
(i) selected from the group consisting of Cas 9, Cas3, Cas8a2, Cas8b, Cas8c, Cas10, Cas11, Cas12, Cas12a or Cpf1, Cas13, Cas13a, C2c1, C2c3, and C2c2,
(ii) a class 2 Cas nuclease, optionally a type II, type V, or type VI Cas nuclease,
(iii) Cas 9. optionally Cas9 of Streptococcus pyogenes (SpCas9), Staphylococcus aureus Cas9 (SaCas9), Streptococcus thermophilus (StCas9), Neisseria meningitidis (NmCas9), Francisella novicida (FnCas9), Campylobacter jejuni (CjCas9), Streptococcus canis (ScCas9), Staphylococcus auricularis (SauriCas9), or any engineered variants thereof, including SaCas9-HF, SpCas9-HF1, KKHSaCas9, eSpCas9, HypaCas9, FokI-Fused dCas9, xCas9, SpRY (variant of SpCas9), and SpG (variant of SpCas9); and/or
(iv) Cas9 comprising any one of SEQ ID NOS: 150-161; and
(b) a guide RNA (gRNA) comprising a target-complementary sequence which is complementary to a target sequence within the at least one SCD-associated gene and/or a promoter or enhancer thereof, or a nucleic acid encoding said gRNA,
wherein:
(i) the gRNA is a single guide RNA (sgRNA) comprising (i-1) a crRNA sequence comprising the target-complementary sequence and a crRNA backbone sequence and (i-2) a trans-activating CRISPR RNA (tracrRNA) sequence in a single strand, optionally wherein the crRNA sequence and the tracrRNA sequence are linked via a linker optionally comprising SEQ ID NO: 139, further optionally wherein the gRNA comprises the target-complementary sequence followed by a sgRNA backbone sequence of any of SEQ ID NOS: 141-144, optionally wherein the sgRNA backbone sequence is followed by one or more uracils, further optionally 1-10 uracils, or
(ii) the gRNA is a dual guide RNA (dgRNA) formed by hybridization between (ii-1) a crRNA sequence comprising the target-complementary sequence and a crRNA backbone sequence and (ii-2) a tracrRNA, optionally wherein the crRNA backbone sequence and the tracrRNA comprise SEQ ID NOS: 145 and 146, respectively, or SEQ ID NOS: 147 and 148, respectively,
optionally wherein the at least one cargo comprises, essentially consists of, or consists of a ribonucleoprotein (RNP), which is a complex of the gRNA and the Cas nuclease, optionally the RNP according to claim 5,
further optionally wherein the RNP is formed by mixing Cas9 and gRNA at an approximately equimolar ratio, optionally for about 5 minutes,
further optionally wherein the pharmaceutical composition or the at least one cargo further comprises a DNA repair template,
yet further optionally wherein the at least one cargo (which comprises the RNP or the RNP and the DNA repair template) encapsulated in the TCV is obtained by:
(i) providing an aqueous solution comprising the TCV, optionally wherein the pH of the aqueous solution is about 3 to about 8, further optionally about 4 to about 7.5; and
(ii) mixing the at least one cargo with the aqueous solution, wherein mixing is effected under conditions suitable for the at least one cargo to be encapsulate within the TCV,
wherein the mixing comprises gentle mixing (optionally repeated manual reciprocation of the TCV-generating fluid in a pipette), micromixing optionally using a staggered herringbone micromixer (SHM) or T-junction or Y-junction mixing, or extrusion, optionally wherein the mixing time is about 0.1 second to about 20 minutes;
wherein the aqueous solution is substantially, essentially, or entirely free of ethanol, methanol, isopropanol, tetrahydrofuran (THF), dimethyl sulfoxide (DMSO), dimethyl formamide (DMF), and acetonitrile (ACN), optionally substantially, essentially, or entirely free of organic solvents and detergents, further optionally substantially, essentially, or entirely free of destabilizing agents,
wherein the mixing is performed substantially, essentially, or entirely free of ethanol, methanol, isopropanol, tetrahydrofuran (THF), dimethyl sulfoxide (DMSO), dimethyl formamide (DMF), and acetonitrile (ACN), optionally substantially, essentially, or entirely free of organic solvents and detergents, further optionally substantially, essentially, or entirely free of destabilizing agents, and
optionally wherein the final ethanol concentration after encapsulation is 5% (v/v) or below, preferably 0.5% (v/v) or below,
further optionally the size of the TCV after encapsulation of the at least one cargo is in a range of about 80 nm to about 1000 nm and/or in arrange of about 100 nm to about 250 nm, at pH of about 7.5,
and optionally wherein the at least one cargo (which comprises the RNP or the RNP and the DNA repair template) encapsulated in the TCV is comprised in a matrix vesicle, which is optionally for gradual release of the TCV.

12. The pharmaceutical composition of claim 11, wherein in (II):
(A) the at least one SCD-associated gene comprises or consists of HBB (such as the sickle cell hemoglobin (HbS) variant of HBB, also known as the βS allele, or the hemoglobin C (HbC) variant of HBB) and/or a promoter or enhancer region of HBB,
optionally wherein the gRNA directs the Cas protein to and hybridize to a target sequence, which is located between nucleotide positions 5225464 to 5227071 of Chromosome 11 (according to Gene Assembly GRCh38.p13, positive or negative strand) and which is optionally within the polynucleotide sequence of SEQ ID NO: 11, 21, or 31 or the sequence complementary thereto, further optionally within or overlapping with exon 1 of HBB,
further optionally wherein the pharmaceutical composition or the at least one cargo further comprises a DNA repair template which allows for a knock-in of or correction to the wildtype HBB gene sequence (SEQ ID NO: 11) or the polynucleotide sequence encoding the wildtype beta-globin amino acid sequence (SEQ ID NO: 1);
(B) the at least one SCD-associated gene comprises or consists of BCL11A and/or the erythroid-enhancer region (EER) of BCL11A and/or a promoter or enhancer region of BCL11A, optionally wherein the gRNA directs the Cas protein to and hybridize to a target sequence, which is located between nucleotide positions 60450520 to 60/553,654 of Chromosome 2 (according to Gene Assembly GRCh38.p13, positive or negative strand) and/or a promoter or enhancer region of BCL11A; and/or
(C) the at least one SCD-associated gene comprises or consists of KLF1 and/or a promoter or enhancer region of KLF1,
optionally wherein the gRNA directs the Cas protein to and hybridize to a target sequence, which is located between nucleotide positions 12884422 to 12887201 of Chromosome 19 (according to Gene Assembly GRCh38.p13, positive or negative strand) and/or a promoter or enhancer region of KLF1,
(D) the at least one SCD-associated gene comprises or consists of HBG1 and/or a promoter or enhancer region of HBG1,
optionally wherein the gRNA directs the Cas protein to and hybridize to a target sequence, which is located between nucleotide positions 5248269 to 5249857 of Chromosome 11 (according to Gene Assembly GRCh38.p14, positive or negative strand) and/or a promoter or enhancer region of HBG1, preferably in the BCL11A-binding site thereof; and/or
(E) the at least one SCD-associated gene comprises or consists of HBG2 and/or a promoter or enhancer region of HBG2,
optionally wherein the gRNA directs the Cas protein to and hybridize to a target sequence, which is located between nucleotide positions 5253188 to 5254781 of Chromosome 11 (according to Gene Assembly GRCh38.p14, positive or negative strand) and/or a promoter or enhancer region of HBG2, preferably in the BCL11A-binding site thereof.

13. The pharmaceutical composition of claim 12, wherein:
in (A), (i) the target sequence is or comprises SEQ ID NO: 24 or the first 17, 18, or 19 nucleotides from the 5′ end of SEQ ID NO: 24, and/or the target-complementary sequence comprises the polynucleotide sequence of SEQ ID NO: 25, or the first 17, 18, or 19 nucleotides thereof from the 3′ end of SEQ ID NO: 25;
(ii) the target sequence is or comprises SEQ ID NO: 44 or the first 17, 18, or 19 nucleotides from the 5′ end of SEQ ID NO: 44 such as SEQ ID NO: 46, and/or the target-complementary sequence comprises the polynucleotide sequence of SEQ ID NO: 45, or the first 17, 18, or 19 nucleotides thereof from the 3′ end of SEQ ID NO: 45 such as SEQ ID NO: 47; and/or
(iii) the target sequence is or comprises SEQ ID NO: 48 or the first 17, 18, or 19 nucleotides from the 5′ end of SEQ ID NO: 48, and/or the target-complementary sequence comprises the polynucleotide sequence of SEQ ID NO: 49, or the first 17, 18, or 19 nucleotides thereof from the 3′ end of SEQ ID NO: 49,
optionally wherein the pharmaceutical composition or the at least one cargo further comprises a DNA repair template, which optionally comprise:
(I) a single-strand oligo DNA nucleotide molecule (ssODN) comprising or consisting of a 5′ homology arm, a central region, and a 3′ homology arm, wherein:
(a) (i) the 5′ homology arm comprises or consists of (i-1) the sequence of SEQ ID NO: 112, (i-2) the sequence corresponding to the first nucleotide to at least the 20th nucleotide (e.g., at least the 30th, such as the 39th, at least the 40th, such as the 49th, or at least the 50th, such as the 50th or the 59th) counting from the 3′-end of SEQ ID NO: 112, (i-3) or a sequence comprising at least one (such as one, two, three, four, five, six, seven, eight, nine, or ten) silent mutation(s) relative to the sequence of (i-1) or (i-2),
(ii) the central region has the sequence of 5′-CTCA-3′, 5′-TTCA-3′, 5′-CTCT-3′, 5′-TTCT-3′, 5′-CTCC-3′, 5′-TTCC-3′, 5′-CTCG-3′, or 5′-TTCG-3′, and
(iii) the 3′ homology arm comprises or consists of (i-1) the sequence of SEQ ID NO: 122, (i-2) the sequence corresponding to the first nucleotide to at least the 20th nucleotide (e.g., at least the 30th, such as the 37th, at least the 40th, such as the 47th, or at least the 50th, such as the 57th) counting from the 5′-end of SEQ ID NO: 122, (i-3) or a sequence comprising at least one (such as one, two, three, four, five, six, seven, eight, nine, or ten) silent mutation(s) relative to the sequence of (iii-1) or (iii-2),
optionally wherein the ssODN comprises the consists of the sequence of any of SEQ ID NOs: 170, 172, 174, 176, and 101-108;
(b) the sequence of the ssODN is fully complementary to any of the ssODNs of (a), optionally wherein the sequence of the ssODN is or comprises any of SEQ ID NOs: 169, 171, 173, and 175; or
(II) a double-strand DNA molecule, which comprises a first strand comprising any of the ssODN sequences of (I) and a second strand complementary to the first strand;
in (B), (i) the target sequence is or comprises SEQ ID NO: 64 or the first 17, 18, or 19 nucleotides from the 5′ end of SEQ ID NO: 64, and/or the target-complementary sequence comprises the polynucleotide sequence of SEQ ID NO: 65, or the first 17, 18, or 19 nucleotides thereof from the 3′ end of SEQ ID NO: 65; and/or
(ii) the target sequence is or comprises SEQ ID NO: 66 or the first 17, 18, or 19 nucleotides from the 5′ end of SEQ ID NO: 66, and/or the target-complementary sequence comprises the polynucleotide sequence of SEQ ID NO: 67 or the first 17, 18, or 19 nucleotides thereof from the 3′ end of SEQ ID NO: 67; and/or
(iii) the target sequence is or comprises SEQ ID NO: 68 or the first 17, 18, or 19 nucleotides from the 5′ end of SEQ ID NO: 68, and/or the target-complementary sequence comprises the polynucleotide sequence of SEQ ID NO: 69 or the first 17, 18, or 19 nucleotides thereof from the 3′ end of SEQ ID NO: 69;
in (C), (i) the target sequence is or comprises SEQ ID NO: 74 or the first 17, 18, or 19 nucleotides from the 5′ end of SEQ ID NO: 74, and/or the target-complementary sequence comprises the polynucleotide sequence of SEQ ID NO: 75 or the first 17, 18, or 19 nucleotides thereof from the 3′ end of SEQ ID NO: 75; and/or
(ii) the target sequence is SEQ ID NO: 76 or the first 17, 18, or 19 nucleotides from the 5′ end of SEQ ID NO: 76, and/or the target-complementary sequence comprises the polynucleotide sequence of SEQ ID NO: 77 or the first 17, 18, or 19 nucleotides thereof from the 3′ end of SEQ ID NO: 77; and/or
in (D) and/or (E), the target sequence is or comprises SEQ ID NO: 84 or the first 17, 18, or 19 nucleotides from the 5′ end of SEQ ID NO: 84, and/or the target-complementary sequence comprises the polynucleotide sequence of SEQ ID NO: 85 or the first 17, 18, or 19 nucleotides thereof from the 3′ end of SEQ ID NO: 85,
and/or wherein the gRNA comprises at least one gRNA according to claim 1.

14. The pharmaceutical composition of any one of claims 6-13, wherein the at least one cargo is capable of altering the expression and comprises, essentially consists of, or consists of a nucleic acid molecule, optionally a ribonucleic acid (RNA), a single or double stranded RNA, a small interfering RNA (siRNA), a short hairpin RNA, a microRNA (miRNA), a messenger RNA (mRNA), a deoxyribonucleic acid (DNA), a double or single stranded DNA, a plasmid DNA, a complementary DNA (cDNA), and/or a locked nucleic acid,
optionally wherein the at least one cargo encapsulated in the TCV is obtained by:
(i) providing an aqueous solution comprising the TCV, optionally wherein the pH of the aqueous solution is about 3 to about 8, further optionally about 4 to about 7.5; and
(ii) mixing the at least one cargo with the aqueous solution, wherein mixing is effected under conditions suitable for the at least one cargo to be encapsulate within the TCV,
wherein the mixing comprises gentle mixing (optionally repeated manual reciprocation of the TCV-generating fluid in a pipette), micromixing optionally using a staggered herringbone micromixer (SHM) or T-junction or Y-junction mixing, or extrusion, optionally wherein the mixing time is about 0.1 second to about 20 minutes;
wherein the aqueous solution is substantially, essentially, or entirely free of ethanol, methanol, isopropanol, tetrahydrofuran (THF), dimethyl sulfoxide (DMSO), dimethyl formamide (DMF), and acetonitrile (ACN), optionally substantially, essentially, or entirely free of organic solvents and detergents, further optionally substantially, essentially, or entirely free of destabilizing agents,
wherein the mixing is performed substantially, essentially, or entirely free of ethanol, methanol, isopropanol, tetrahydrofuran (THF), dimethyl sulfoxide (DMSO), dimethyl formamide (DMF), and acetonitrile (ACN), optionally substantially, essentially, or entirely free of organic solvents and detergents, further optionally substantially, essentially, or entirely free of destabilizing agents, and
optionally wherein the final ethanol concentration after encapsulation is 5% (v/v) or below, preferably 0.5% (v/v) or below.

15. The pharmaceutical composition of any one of claims 6-14, wherein:
(A) the pharmaceutical composition comprises at least another cargo, which is encapsulated in the TCV encapsulating said at least one cargo or in a different TCV, optionally wherein the at least another cargo is according to the at least one cargo of any one of claims 6-14;
(B) the pharmaceutical composition further comprises at least one agent that promotes stem cell mobilization, optionally selected from the group consisting of granulocyte colony-stimulating factor (G-CSF), granulocyte-macrophage colony-stimulating factor (GM-CSF), Plerixafor, stem cell factor (SCF), CXCR4 antagonists (e.g., POL6326, BKT-140, TG-0054), CXCL12 neutralizers (e.g., NOX-A12), Sphingosine-1-phosphate (SIP) antagonists (e.g., SEW2871), vascular cell adhesion molecule-1/Very Late Antigen 4 (VCAM/VLA-4) inhibitors (e.g., BIO 5192), parathyroid hormone, protease inhibitors (e.g., Bortezomib), Grop (e.g., SB-251353), and hypoxia inducible factor (HIF) stabilizers (e.g., FG-4497);
(C) the pharmaceutical composition further comprises at least one agent that promotes erythropoiesis, which optionally comprises:
(i) an agent selected from the group consisting of SCF, GM-CSF, interleukin-3 (IL-3), interleukin-9 (IL-9), erythropoietin (EPO) (or an engineered EPO or EPO mimetic), TGF-beta, growth differentiating factor 11 (GDF11), Activin A, Transferrin (Tf), ferritin, ferroportin, hepcidin, vitamin B12, folic acid, and copper,
(ii) an agent selected from the group consisting of GATA-1, STAT5A, STAT5B, MCL-1, BCL-xL, and HSP70, a RNA or DNA encoding thereof, optionally wherein the agent is encapsulated in the TCV encapsulating said at least one cargo or in a different TCV; and/or
(iii) an inhibitor or silencer of a negative regulator of erythropoiesis, optionally wherein the negative regulator is selected from the group consisting of inhibin, TGF-beta, BID (a member of the BCL-2 family), Fas ligand, Fas, and caspases, optionally wherein the agent is encapsulated in the TCV encapsulating said at least one cargo or in a different TCV; and/or
(D) the TCV comprises at least one targeting moiety which allows the TCV to carry the at least one cargo preferentially into one or more target cells, optionally wherein the one or more target cells comprise hematopoietic stem cells (HSCs), hematopoietic stem and progenitor cells (HSPCs), multipotent progenitor cells (MPPs), common myeloid progenitors (CMPs), megakaryocyte-erythroid progenitors (MEPs), hematopoietic progenitor cells (HPCs), erythroid progenitors (e.g., burst-forming unit erythroid cells (BFU-Es), colony-forming unit erythroid cells (CFU-Es)), proerythroblasts, erythroblasts (basophilic erythroblasts, early erythroblasts (e.g., type I, type II), polychromatic erythroblasts, intermediate erythroblasts, acidophilic erythroblasts, late erythroblasts, normoblasts, reticulocytes (before nucleus expulsion), or any combinations thereof, preferably HSCs and/or HSPCs, optionally wherein the targeting moiety targets CD34.

16. A method of effecting gene editing and/or gene expression alteration in one or more target cells in vivo in a subject in need thereof, the method comprising injecting the pharmaceutical composition of any one of claims 6-15 into the bone marrow of the subject,
wherein the one or more target cells comprise HSCs, HSPCs, MPPs, CMPs, MEPs, HPCs, erythroid progenitors (e.g., BFU-Es, CFU-Es), proerythroblasts, erythroblasts (basophilic erythroblasts, early erythroblasts (e.g., type I, type II), polychromatic erythroblasts, intermediate erythroblasts, acidophilic erythroblasts, late erythroblasts, normoblasts, reticulocytes (before nucleus expulsion), or any combinations thereof, preferably HSCs and/or HSPCs.
optionally the subject has or has a risk of developing SCD, which is optionally SCA, HbSC, or HbS β-thalassaemia.

17. The method of claim 16, which comprises one or more of the following features:
(i) the pharmaceutical composition is according to the pharmaceutical composition of any of one of claims 6-15, and comprises per mL about 300 pmol to about 30000 pmol, optionally about 500 to about 10000 pmol, about 1000 to about 5000 pmol, about 2000 to about 4000 pmol, about 2500 to about 3000 pmol, or about 2700 pmol of the RNP or the nucleic acid molecule;
(ii) the injecting comprising injecting the pharmaceutical composition in a continuous flow of about 25 mL to 125 mL per minute, optionally about 25 mL to 50 mL per minute, about 50 mL to 100 mL per minute, about 100 mL to 125 mL per minute, about 40 mL to about 80 mL per minute, or about 50 mL to about 70 mL per minute;
(iii) the injecting is a slow bolus push using an instrument with an intraosseous device or needle optionally having a needle length of about 50 to about 100 mm or about 70 to about 80 nm;
(iv) the injecting is effected, optionally two or more times, to reach a minimum of about 10%, about 15%, about 20%, about 30%, or an about final 15-30% or about final 20-40% HSCs and HSPCs with successful gene editing and/or gene expression alteration among the total HSCs and HSPCs in the bone marrow;
(v) the injecting is effected two or more times, optionally about 3-5 time, optionally about once a week, about every 2 weeks, or about every 3 weeks, about once a month, about every 3 months, about every 6 months, or about once per year;
(vi) the bone marrow is of tibia, femur, sternum, skull, ribs, pelvis (e.g., iliac), or any combinations thereof; and/or
(vii) the subject (a) is in the immediate post-natal period, optionally about 6 weeks old or younger, (b) is about 3 month old or younger, (c) still comprises sufficient amount of fetal hemoglobin (HbF) relative to adult hemoglobin (HbA) (e.g., HbF:HbA is about 2:1, about 1:1, about 1:2, about 1:3, about 1:4, about 1:5, or about 1:10), and/or (d) has not fully developed SCD and is prior to manifesting a symptom or complication.

18. A method of effecting gene editing and/or gene expression alteration in one or more target cells in vivo in a subject in need thereof, the method comprising:
(I) administering, optionally intravenously, to the subject at least one agent that promotes stem cell mobilization (from the bone marrow to the peripheral circulation), optionally selected from the group consisting of G-CSF (filgrastim), GM-CSF, Plerixafor, SCF, CXCR4 antagonists (e.g., POL6326, BKT-140, TG-0054), CXCL12 neutralizers (e.g., NOX-A12), Sphingosine-1-phosphate (SIP) antagonists (e.g., SEW2871), VCAM/VLA-4 inhibitors (e.g., BIO 5192), parathyroid hormone, protease inhibitors (e.g., Bortezomib), Grop (e.g., SB-251353), and hypoxia inducible factor (HIF) stabilizers (e.g., FG-4497), and
(II) injecting, optionally intravenously, the pharmaceutical composition of any one of claims 6-15 into the peripheral circulation of the subject,
wherein the one or more target cells comprise HSCs, HSPCs, MPPs, CMPs, MEPs, HPCs, erythroid progenitors (e.g., BFU-Es, CFU-Es), proerythroblasts, erythroblasts (basophilic erythroblasts, early erythroblasts (e.g., type I, type II), polychromatic erythroblasts, intermediate erythroblasts, acidophilic erythroblasts, late erythroblasts, normoblasts, reticulocytes (before nucleus expulsion), or any combinations thereof, preferably HSCs and/or HSPCs,
optionally the subject has or has a risk of developing SCD, which is optionally SCA, HbSC, or HbS β-thalassaemia.

19. The method of claim 18, which comprises one or more of the following features:
(i) said administering comprises intravenous administration of G-CSF followed by intravenous administration of plerixafor prior to said injecting, optionally wherein:
(a) the dosing of G-CSF is about 5-30 μg/kg/day, preferably about 10 μg/kg/day, for about 3-5 days, preferably 4 days,
(b) the dosing of plerixafor starts once the peripheral blood CD34⁺ cells are <20 cells/μL and/or on the day of the last G-CSF administration or the following day, and/or
(c) the dosing of plerixafor is about 0.1-0.5 mg/kg, preferably about 0.2-0.3 mg/kg or about 0.24 mg/kg;
(ii) the pharmaceutical composition is according to the pharmaceutical composition of any of one claims 6-15 and comprises per mL about 300 pmol to about 30000 pmol, optionally about 500 to about 10000 pmol, about 1000 to about 5000 pmol, about 2000 to about 4000 pmol, about 2500 to about 3000 pmol, or about 2700 pmol of the RNP or the nucleic acid molecule;
(iii) said injecting:
(a) starts once the peripheral blood CD34⁺ cells are 60 cells/μL or more,
(b) is a single injection, optionally about 3-7 days, about 4-6 days, or about 5 days after the last plerixafor administration, and/or
(c) occurs once daily for one week following the last plerixafor administration;
(iv) the injecting comprising injecting the pharmaceutical composition in a continuous flow of about 25 mL to 125 mL per minute, optionally about 25 mL to 50 mL per minute, about 50 mL to 100 mL per minute, about 100 mL to 125 mL per minute, about 40 mL to about 80 mL per minute, or about 50 mL to about 70 mL per minute;
(v) the combination of said administering and said injecting is effected, optionally two or more times, to reach a minimum of about 10%, about 15%, about 20%, about 30%, or an about final 15-30% or about final 20-40% HSCs and HSPCs with successful gene editing and/or gene expression alteration among the total HSCs and HSPCs in the peripheral circulation;
(vi) the combination of said administering and said injecting is effected, optionally two or more times, to reach a minimum of about 10%, about 15%, about 20%, about 30%, or an about final 20-30% increase in the peripheral HSCs and HSPCs expressing HbF, or a minimum of about 10%, about 15%, about 20%, about 30%, or an about final 20-30% increase in the total HbF expression levels in the total HSCs and HSPCs in the peripheral circulation, optionally wherein the SCD-associated gene is BCL11A, HBG1, HBG2, or KLF1;
(vii) the combination of said administering and said injecting is effected two or more times, optionally about 3-5 time, optionally about once a week, about every 2 weeks, or about every 3 weeks, about once a month, about every 3 months, about every 6 months, or about once per year;
(viii) the subject (a) is in the immediate post-natal period, optionally about 6 weeks old or younger, (b) is about 3 month old or younger, (c) still comprises sufficient amount of HbF relative to HbA (e.g., HbF:HbA is about 2:1, about 1:1, about 1:2, about 1:3, about 1:4, about 1:5, or about 1:10), and/or (d) has not fully developed SCD and is prior to manifesting a symptom or complication.

20. A method of preventing, ameliorating, or treating SCD, which is optionally SCA, HbSC, or HbS β-thalassaemia, in a subject in need thereof, the method comprises the method according to claim 16 or 17 and/or the method according to claim 18 or 19,
optionally the effect of the method is evaluated based on:
(i) % HSCs and HSPCs in the blood with successful gene editing and/or gene expression alteration;
(ii) the number of HSCs and HSPCs in the blood with successful gene editing and/or gene expression alteration;
(iii) % HSCs and HSPCs expressing HbF, optionally wherein the SCD-associated gene is BCL11A or KLF1;
(iv) the number of HSCs and HSPCs expressing HbF, optionally wherein the SCD-associated gene is BCL11A, HBG1, HBG2, or KLF1;
(v) the expression level of the at least one SCD-associated gene or gene product or molecule, optionally beta-globin, beta-globin variant (HbS variant or HbC variant), gamma-globin 1, gamma-globin 2, HbF, HbA, BCL11A, and/or KLF1; and/or
(iv) changes in the symptom optionally pain, swelling of hands and feet, infection frequency, growth, and/or symptoms associated with vision,
and optionally wherein the method further comprises:
(A) administering at least one agent that promotes erythropoiesis, which optionally comprises:
(a) an agent selected from the group consisting of SCF, GM-CSF, IL-3, IL-9, EPO (or an engineered EPO or EPO mimetic), TGF-beta, GDF11, Activin A, Tf, ferritin, ferroportin, hepcidin, vitamin B12, folic acid, and copper,
(b) an agent selected from the group consisting of GATA-1, STAT5A, STAT5B, MCL-1, BCL-xL, and HSP70, a RNA or DNA encoding thereof, optionally wherein the agent is encapsulated in the TCV encapsulating said at least one cargo or in a different TCV, and/or
(c) an inhibitor or silencer of a negative regulator of erythropoiesis, optionally wherein the negative regulator is selected from the group consisting of inhibin, TGF-beta, BID (a member of the BCL-2 family), Fas ligand, Fas, and caspases, optionally wherein the agent is encapsulated in the TCV encapsulating said at least one cargo or in a different TCV; and/or
(B) administering at least one other agent for treating SCD, which optionally comprises hydroxyurea, L-glutamine oral powder, crizanlizumab, a general pain medication, voxelotor, or any combination thereof.

21. A method of manufacturing the pharmaceutical composition of claim 11(II), 12, or 13, comprising:
(a) providing an aqueous solution comprising the TCV, optionally wherein the pH of the aqueous solution is about 3 to about 8, further optionally about 3.5 to about 7.5, about 3.5 to about 5.5, or about 4; and
(b) mixing the at least one cargo with the aqueous solution under conditions suitable for the at least one cargo to be encapsulate within the TCV,
optionally wherein the at least one cargo comprises the RNP of claim 5,
and optionally wherein the mixing is for about 0.1 second to about 20 minutes, optionally via gentle mixing (optionally repeated manual reciprocation of the TCV-generating fluid in a pipette), optionally via micromixing, further optionally using a staggered herringbone micromixer (SHM) or T-junction or Y-junction mixing, or extrusion.


SEQUENCE LISTING (184 sequences)

SEQ ID NO: 1 - protein, 147 aa, synthetic construct
  source 1..147: note = Human hemoglobin subunit beta (beta-globin or HBB), wild-type
MVHLTPEEKSAVTALWGKVNVDEVGGEALGRLLVVYPWTQRFFESFGDLSTPDAVMGNPKVKAHGKKVLGAFSDGLAHLDNLKGTFATLSELHCDKLHVDPENFRLLGNVLVCVLAHHFGKEFTPPVQAAYQKVVAGVANALAHKYH

SEQ ID NO: 2 - protein, 147 aa, synthetic construct [sequence omitted]

SEQ ID NO: 3 - protein, 147 aa, synthetic construct [sequence omitted]

SEQ ID NO: 4-5 - skipped (2 entries)

SEQ ID NO: 6 - protein, 835 aa, synthetic construct
  source 1..835: note = Human BAF chromatin remodeling complex subunit BCL11A (BCL11A)
MSRRKQGKPQHLSKREFSPEPLEAILTDDEPDHGPLGAPEGDHDLLTCGQCQMNFPLGDILIFIEHKRKQCNGSLCLEKAVDKPPSPSPIEMKKASNPVEVGIQVTPEDDDCLSTSSRGICPKQEHIADKLLHWRGLSSPRSAHGALIPTPGMSAEYAPQGICKDEPSSYTCTTCKQPFTSAWFLLQHAQNTHGLRIYLESEHGSPLTPRVGIPSGLGAECPSQPPLHGIHIADNNPFNLLRIPGSVSREASGLAEGRFPPTPPLFSPPPRHHLDPHRIERLGAEEMALATHHPSAFDRVLRLNPMAMEPPAMDFSRRLRELAGNTSSPPLSPGRPSPMQRLLQPFQPGSKPPFLATPPLPPLQSAPPPSQPPVKSKSCEFCGKTFKFQSNLVVHRRSHTGEKPYKCNLCDHACTQASKLKRHMKTHMHKSSPMTVKSDDGLSTASSPEPGTSDLVGSASSALKSVVAKFKSENDPNLIPENGDEEEEEDDEEEEEEEEEEEEELTESERVDYGFGLSLEAARHHENSSRGAVVGVGDESRALPDVMQGMVLSSMQHFSEAFHQVLGEKHKRGHLAEAEGHRDTCDEDSVAGESDRIDDGTVNGRGCSPGESASGGLSKKLLLGSPSSLSPFSKRIKLEKEFDLPPAAMPNTENVYSQWLAGYAASRQLKDPFLSFGDSRQSPFASSSEHSSENGSLRFSTPPGELDGGISGRSGTGSGGSTPHISGPGPGRPSSKEGRRSDTCEYCGKVFKNCSNLTVHRRSHTGERPYKCELCNYACAQSSKLTRHMKTHGQVGKDVYKCEICKMPFSVYSTLEKHMKKWHSDRVLNNDIKTE

SEQ ID NO: 7 - protein, 362 aa, synthetic construct [sequence omitted]

SEQ ID NO: 8 - protein, 147 aa, synthetic construct
  source 1..147: note = Human hemoglobin subunit gamma 1 (A-gamma-globin or HBG1)
MGHFTEEDKATITSLWGKVNVEDAGGETLGRLLVVYPWTQRFFDSFGNLSSASAIMGNPKVKAHGKKVLTSLGDAIKHLDDLKGTFAQLSELHCDKLHVDPENFKLLGNVLVTVLAIHFGKEFTPEVQASWQKMVTAVASALSSRYH

SEQ ID NO: 9 - protein, 147 aa, synthetic construct
  source 1..147: note = Human hemoglobin subunit gamma 2 (G-gamma-globin or HBG2)
MGHFTEEDKATITSLWGKVNVEDAGGETLGRLLVVYPWTQRFFDSFGNLSSASAIMGNPKVKAHGKKVLTSLGDAIKHLDDLKGTFAQLSELHCDKLHVDPENFKLLGNVLVTVLAIHFGKEFTPEVQASWQKMVTGVASALSSRYH

SEQ ID NO: 10 - skipped

SEQ ID NO: 11 - DNA, 1608 bp, synthetic construct
  source 1..1608: mol_type = other DNA; note = Human hemoglobin subunit beta, wild-type, encoded on Chromosome 11 (11p15.4; Assembly GRCh38.p13) from positions 5225464 to 5227071 of Chromosome 11 (1608 nt including the UTRs)
acatttgcttctgacacaactgtgttcactagcaacctcaaacagacaccatggtgcatctgactcctgaggagaagtctgccgttactgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 12 - DNA, 1421 bp, synthetic construct
  source 1..1421: mol_type = other DNA; note = Human hemoglobin subunit beta ORF, wild-type, from positions 5225601 to 5227021 of Chromosome 11; containing positions 5226929-5227021 (Exon 1 minus 5 UTR), positions 5226577-5226798 (Exon 2), and positions 5225601-5225726 (Exon 3 minus 3 UTR)
atggtgcatctgactcctgaggagaagtctgccgttactgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcaccccaccagtgcaggctgcctatcagaaagtggtggctggtgtggctaatgccctggcccacaagtatcac

SEQ ID NO: 13 - DNA, 444 bp, synthetic construct
  source 1..444: mol_type = other DNA; note = Human hemoglobin subunit beta cDNA, wild-type
atggtgcatctgactcctgaggagaagtctgccgttactgccctgtggggcaaggtgaacgtggatgaagttggtggtgaggccctgggcaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcaggctcctgggcaacgtgctggtctgtgtgctggcccatcactttggcaaagaattcaccccaccagtgcaggctgcctatcagaaagtggtggctggtgtggctaatgccctggcccacaagtatcactaa

SEQ ID NO: 14-20 - skipped (7 entries)

SEQ ID NO: 21 - DNA, 1608 bp, synthetic construct
  source 1..1608: mol_type = other DNA; note = Human hemoglobin subunit beta, Sickle Hb variant (also referred to as betaS allele), encoded on Chromosome 11 (11p15.4; Assembly GRCh38.p13) from positions 5225464 to 5227071 of Chromosome 11 (1608 nt including the UTRs)
acatttgcttctgacacaactgtgttcactagcaacctcaaacagacaccatggtgcatctgactcctgtggagaagtctgccgttactgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 22 - DNA, 1421 bp, synthetic construct
  source 1..1421: mol_type = other DNA; note = Human hemoglobin subunit beta ORF, Sickle Hb variant (also referred to as betaS allele), from positions 5225601 to 5227021 of Chromosome 11
atggtgcatctgactcctgtggagaagtctgccgttactgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcaccccaccagtgcaggctgcctatcagaaagtggtggctggtgtggctaatgccctggcccacaagtatcac

SEQ ID NO: 23 - DNA, 444 bp, synthetic construct
  source 1..444: mol_type = other DNA; note = Human hemoglobin subunit beta cDNA, Sickle Hb variant (also referred to as betaS allele)
atggtgcatctgactcctgtggagaagtctgccgttactgccctgtggggcaaggtgaacgtggatgaagttggtggtgaggccctgggcaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcaggctcctgggcaacgtgctggtctgtgtgctggcccatcactttggcaaagaattcaccccaccagtgcaggctgcctatcagaaagtggtggctggtgtggctaatgccctggcccacaagtatcactaa

SEQ ID NO: 24 - DNA, 20 bp, synthetic construct
  source 1..20: mol_type = other DNA; note = Human HBB HbS variant CRISPR/Cas target sequence 1
gtggagaagtctgccgttac

SEQ ID NO: 25 - RNA, 20 nt, synthetic construct
  source 1..20: mol_type = other RNA; note = gRNA target-complementary sequence, complementary to Human HBB HbS variant CRISPR/Cas target sequence 1
gtaacggcagacttctccac

SEQ ID NO: 26-30 - skipped (5 entries)

SEQ ID NO: 31 - DNA, 1608 bp, synthetic construct
  source 1..1608: mol_type = other DNA; note = Human hemoglobin subunit beta, Hb C variant (also referred to as betaC allele), encoded on Chromosome 11 (11p15.4; Assembly GRCh38.p13) from positions 5225464 to 5227071 of Chromosome 11 (1608 nt including the UTRs)
acatttgcttctgacacaactgtgttcactagcaacctcaaacagacaccatggtgcatctgactcctaaggagaagtctgccgttactgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 32 - DNA, 1421 bp, synthetic construct
  source 1..1421: mol_type = other DNA; note = Human hemoglobin subunit beta ORF, Hb C variant (also referred to as betaC allele), from positions 5225601 to 5227021 of Chromosome 11
atggtgcatctgactcctaaggagaagtctgccgttactgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcaccccaccagtgcaggctgcctatcagaaagtggtggctggtgtggctaatgccctggcccacaagtatcac

SEQ ID NO: 33 - DNA, 444 bp, synthetic construct
  source 1..444: mol_type = other DNA; note = Human hemoglobin subunit beta cDNA, Sickle Hb C variant (also referred to as betaC allele)
atggtgcatctgactcctaaggagaagtctgccgttactgccctgtggggcaaggtgaacgtggatgaagttggtggtgaggccctgggcaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcaggctcctgggcaacgtgctggtctgtgtgctggcccatcactttggcaaagaattcaccccaccagtgcaggctgcctatcagaaagtggtggctggtgtggctaatgccctggcccacaagtatcactaa

SEQ ID NO: 34-40 - skipped (7 entries)

SEQ ID NO: 41 - DNA, 21 bp, synthetic construct
  source 1..21: mol_type = other DNA; note = Human HBB exon 1 forward primer
tgcttaccaagctgtgattcc

SEQ ID NO: 42 - DNA, 20 bp, synthetic construct
  source 1..20: mol_type = other DNA; note = Human HBB exon 1 reverse primer
cactcagtgtggcaaaggtg

SEQ ID NO: 43 - skipped

SEQ ID NO: 44 - DNA, 20 bp, synthetic construct
  source 1..20: mol_type = other DNA; note = Human HBB exon 1 CRISPR/Cas target sequence 1
ttactgccctgtggggcaag

SEQ ID NO: 45 - RNA, 20 nt, synthetic construct
  source 1..20: mol_type = other RNA; note = gRNA target-complementary sequence, complementary to Human HBB exon 1 CRISPR/Cas target sequence 1
cttgccccacagggcagtaa

SEQ ID NO: 46 - DNA, 18 bp, synthetic construct
  source 1..18: mol_type = other DNA; note = Human HBB exon 1 CRISPR/Cas target sequence 2
ttactgccctgtggggca

SEQ ID NO: 47 - RNA, 18 nt, synthetic construct
  source 1..18: mol_type = other RNA; note = gRNA target-complementary sequence, complementary to Human HBB exon 1 CRISPR/Cas target sequence 2
tgccccacagggcagtaa

SEQ ID NO: 48 - DNA, 20 bp, synthetic construct
  source 1..20: mol_type = other DNA; note = Human HBB exon 1 CRISPR/Cas target sequence 3
caggagtcagatgcaccatg

SEQ ID NO: 49 - RNA, 20 nt, synthetic construct
  source 1..20: mol_type = other RNA; note = gRNA target-complementary sequence, complementary to Human HBB exon 1 CRISPR/Cas target sequence 3
catggtgcatctgactcctg

SEQ ID NO: 50-53 - skipped (4 entries)

SEQ ID NO: 54 - DNA, 20 bp, synthetic construct
  source 1..20: mol_type = other DNA; note = Luciferase CRISPR/Cas target sequence 1
acccaacggacatttcgaag

SEQ ID NO: 55 - RNA, 20 nt, synthetic construct
  source 1..20: mol_type = other RNA; note = gRNA luciferase target-complementary sequencem complementary to Luciferase CRISPR/Cas target sequence 1
cttcgaaatgtccgttgggt

SEQ ID NO: 56-60 - skipped (5 entries)

SEQ ID NO: 61 - DNA, 21 bp, synthetic construct
  source 1..21: mol_type = other DNA; note = Human BCL11A EER forward primer
tccaaactctcaaaccacagg

SEQ ID NO: 62 - DNA, 20 bp, synthetic construct
  source 1..20: mol_type = other DNA; note = Human BCL11A EER reverse primer
ggcaagtcagttgggaacac

SEQ ID NO: 63 - skipped

SEQ ID NO: 64 - DNA, 20 bp, synthetic construct
  source 1..20: mol_type = other DNA; note = Human BCL11A EER CRISPR/Cas target sequence 1 (BCL11A genomic antisense sequence)
gtgataaaagcaactgttag

SEQ ID NO: 65 - RNA, 20 nt, synthetic construct
  source 1..20: mol_type = other RNA; note = gRNA target-complementary sequence, complementary to Human BCL11A EER CRISPR/Cas target sequence 1
ctaacagttgcttttatcac

SEQ ID NO: 66 - DNA, 20 bp, synthetic construct
  source 1..20: mol_type = other DNA; note = Human BCL11A EER CRISPR/Cas target sequence 2 (BCL11A genomic antisense sequence)
ggagcctgtgataaaagcaa

SEQ ID NO: 67 - RNA, 20 nt, synthetic construct
  source 1..20: mol_type = other RNA; note = gRNA target-complementary sequence, complementary to Human BCL11A EER CRISPR/Cas target sequence 2
ttgcttttatcacaggctcc

SEQ ID NO: 68 - DNA, 20 bp, synthetic construct
  source 1..20: mol_type = other DNA; note = Human BCL11A EER CRISPR/Cas target sequence 3 (BCL11A genomic antisense sequence)
aacccttcctggagcctgtg

SEQ ID NO: 69 - RNA, 20 nt, synthetic construct
  source 1..20: mol_type = other RNA; note = gRNA target-complementary sequence, complementary to Human BCL11A EER CRISPR/Cas target sequence 3
cacaggctccaggaagggtt

SEQ ID NO: 70-73 - skipped (4 entries)

SEQ ID NO: 74 - DNA, 20 bp, synthetic construct
  source 1..20: mol_type = other DNA; note = Human KLF1 CRISPR/Cas target sequence 1 (KLF1 genomic antisense sequence)
cttgcgcgcccacgaacgtc

SEQ ID NO: 75 - RNA, 20 nt, synthetic construct
  source 1..20: mol_type = other RNA; note = gRNA target-complementary sequence, complementary to Human KLF1 CRISPR/Cas target sequence 1
gacgttcgtgggcgcgcaag

SEQ ID NO: 76 - DNA, 20 bp, synthetic construct
  source 1..20: mol_type = other DNA; note = Human KLF1 CRISPR/Cas target sequence 2 (KLF1 genomic antisense sequence)
agcgcgcgaatctccagccg

SEQ ID NO: 77 - RNA, 20 nt, synthetic construct
  source 1..20: mol_type = other RNA; note = gRNA target-complementary sequence, complementary to Human KLF1 CRISPR/Cas target sequence 2
cggctggagattcgcgcgct

SEQ ID NO: 78-80 - skipped (3 entries)

SEQ ID NO: 81 - DNA, 20 bp, synthetic construct
  source 1..20: mol_type = other DNA; note = Human HBG1 promoter region forward primer
tctcccaaggaagtcagcac

SEQ ID NO: 82 - DNA, 27 bp, synthetic construct
  source 1..27: mol_type = other DNA; note = Human HBG1 promoter region reverse primer
cttagaaaccactgctaactgaaagag

SEQ ID NO: 83 - skipped

SEQ ID NO: 84 - DNA, 20 bp, synthetic construct
  source 1..20: mol_type = other DNA; note = Human HBG1 and HBG2 promoter region CRISPR/Cas target sequence 1
tgaccaatagccttgacaag

SEQ ID NO: 85 - RNA, 20 nt, synthetic construct
  source 1..20: mol_type = other RNA; note = gRNA target-complementary sequence, complementary to Human HBG1 and HBG2 promoter region CRISPR/Cas target sequence 1
cttgtcaaggctattggtca

SEQ ID NO: 86-90 - skipped (5 entries)

SEQ ID NO: 91 - DNA, 20 bp, synthetic construct
  source 1..20: mol_type = other DNA; note = Human HBG2 promoter region forward primer
cagaggacaggttgccaaag

SEQ ID NO: 92 - DNA, 28 bp, synthetic construct
  source 1..28: mol_type = other DNA; note = Human HBG2 promoter region reverse primer
ccaatgcttactaaatgagactaagacg

SEQ ID NO: 93-100 - skipped (8 entries)

SEQ ID NO: 101 - DNA, 121 bp, synthetic construct
  source 1..121: mol_type = other DNA; note = Human beta-globin (WT)-encoding ssODN sequence 1
tcaccaccaacttcatccacgttcaccttgccccacagggcagtaacggcagacttctcctcaggagtcagatgcaccatggtgtctgtttgaggttgctagtgaacacagttgtgtcaga

SEQ ID NO: 102 - DNA, 121 bp, synthetic construct
  source 1..121: mol_type = other DNA; note = Human beta-globin (WT)-encoding ssODN sequence 2
tcaccaccaacttcatccacgttcaccttgccccacagggcagtaacggcagacttctcttcaggagtcagatgcaccatggtgtctgtttgaggttgctagtgaacacagttgtgtcaga

SEQ ID NO: 103 - DNA, 121 bp, synthetic construct
  source 1..121: mol_type = other DNA; note = Human beta-globin (WT)-encoding ssODN sequence 3
tcaccaccaacttcatccacgttcaccttgccccacagggcagtaacggcagacttctcctctggagtcagatgcaccatggtgtctgtttgaggttgctagtgaacacagttgtgtcaga

SEQ ID NO: 104 - DNA, 121 bp, synthetic construct
  source 1..121: mol_type = other DNA; note = Human beta-globin (WT)-encoding ssODN sequence 4
tcaccaccaacttcatccacgttcaccttgccccacagggcagtaacggcagacttctcttctggagtcagatgcaccatggtgtctgtttgaggttgctagtgaacacagttgtgtcaga

SEQ ID NO: 105 - DNA, 121 bp, synthetic construct
  source 1..121: mol_type = other DNA; note = Human beta-globin (WT)-encoding ssODN sequence 5
tcaccaccaacttcatccacgttcaccttgccccacagggcagtaacggcagacttctcctccggagtcagatgcaccatggtgtctgtttgaggttgctagtgaacacagttgtgtcaga

SEQ ID NO: 106 - DNA, 121 bp, synthetic construct
  source 1..121: mol_type = other DNA; note = Human beta-globin (WT)-encoding ssODN sequence 6
tcaccaccaacttcatccacgttcaccttgccccacagggcagtaacggcagacttctcttccggagtcagatgcaccatggtgtctgtttgaggttgctagtgaacacagttgtgtcaga

SEQ ID NO: 107 - DNA, 121 bp, synthetic construct
  source 1..121: mol_type = other DNA; note = Human beta-globin (WT)-encoding ssODN sequence 7
tcaccaccaacttcatccacgttcaccttgccccacagggcagtaacggcagacttctcctcgggagtcagatgcaccatggtgtctgtttgaggttgctagtgaacacagttgtgtcaga

SEQ ID NO: 108 - DNA, 121 bp, synthetic construct
  source 1..121: mol_type = other DNA; note = Human beta-globin (WT)-encoding ssODN sequence 8
tcaccaccaacttcatccacgttcaccttgccccacagggcagtaacggcagacttctcttcgggagtcagatgcaccatggtgtctgtttgaggttgctagtgaacacagttgtgtcaga

SEQ ID NO: 109-110 - skipped (2 entries)

SEQ ID NO: 111 - DNA, 60 bp, synthetic construct
  source 1..60: mol_type = other DNA; note = 5 homology arm (60 nt) of SEQ ID NO: 101, 103, 105, and 107
tcaccaccaacttcatccacgttcaccttgccccacagggcagtaacggcagacttctcc

SEQ ID NO: 112 - DNA, 59 bp, synthetic construct
  source 1..59: mol_type = other DNA; note = 5 homology arm (59 nt) of SEQ ID NO: 102, 104, 106, and 108
tcaccaccaacttcatccacgttcaccttgccccacagggcagtaacggcagacttctc

SEQ ID NO: 113-120 - skipped (8 entries)

SEQ ID NO: 121 - DNA, 60 bp, synthetic construct
  source 1..60: mol_type = other DNA; note = 3 homology arm (60 nt) of SEQ ID NO: 101, 102
caggagtcagatgcaccatggtgtctgtttgaggttgctagtgaacacagttgtgtcaga

SEQ ID NO: 122 - DNA, 58 bp, synthetic construct
  source 1..58: mol_type = other DNA; note = 3 homology arm (58 nt) of SEQ ID NO: 103, 104, 105, 106, 107, and 108
ggagtcagatgcaccatggtgtctgtttgaggttgctagtgaacacagttgtgtcaga

SEQ ID NO: 123-130 - skipped (8 entries)

SEQ ID NO: 131 - RNA, 12 nt, synthetic construct
  source 1..12: mol_type = other RNA; note = crRNA flagpole sequence (option 1)
gttttagagcta

SEQ ID NO: 132 - RNA, 12 nt, synthetic construct
  source 1..12: mol_type = other RNA; note = crRNA flagpole sequence (option 2)
gtttaagagcta

SEQ ID NO: 133-135 - skipped (3 entries)

SEQ ID NO: 136 - RNA, 13 nt, synthetic construct
  source 1..13: mol_type = other RNA; note = tracrRNA flagpole (option 1)
tagcaagttaaaa

SEQ ID NO: 137 - RNA, 13 nt, synthetic construct
  source 1..13: mol_type = other RNA; note = tracrRNA flagpole (option 2)
tagcaagtttaaa

SEQ ID NO: 138 - RNA, 47 nt, synthetic construct
  source 1..47: mol_type = other RNA; note = tracrRNA nuclease binding domain (may be followed by multiple uracils)
taaggctagtccgttatcaacttgaaaaagtggcaccgagtcggtgc

SEQ ID NO: 139-140 - skipped (2 entries)

SEQ ID NO: 141 - RNA, 76 nt, synthetic construct
  source 1..76: mol_type = other RNA; note = sgRNA backbone (3 to targeting sequence) (option 1) (may be followed by multiple uracils)
gttttagagctagaaatagcaagttaaaataaggctagtccgttatcaacttgaaaaagtggcaccgagtcggtgc

SEQ ID NO: 142 - RNA, 76 nt, synthetic construct
  source 1..76: mol_type = other RNA; note = sgRNA backbone (3' to targeting sequence) (option 2) (may be followed by multiple uracils)
gtttaagagctagaaatagcaagtttaaataaggctagtccgttatcaacttgaaaaagtggcaccgagtcggtgc

SEQ ID NO: 143 - RNA, 86 nt, synthetic construct
  source 1..86: mol_type = other RNA; note = sgRNA backbone (3 to targeting sequence) (option 3) (may be followed by multiple uracils)
gttttagagctatgctggaaacagcatagcaagttaaaataaggctagtccgttatcaacttgaaaaagtggcaccgagtcggtgc

SEQ ID NO: 144 - RNA, 86 nt, synthetic construct
  source 1..86: mol_type = other RNA; note = sgRNA backbone (3 to targeting sequence) (option 4) (may be followed by multiple uracils)
gtttaagagctatgctggaaacagcatagcaagtttaaataaggctagtccgttatcaacttgaaaaagtggcaccgagtcggtgc

SEQ ID NO: 145 - RNA, 22 nt, synthetic construct
  source 1..22: mol_type = other RNA; note = dgRNA crRNA backbone (option 1)
gttttagagctatgctgttttg

SEQ ID NO: 146 - RNA, 74 nt, synthetic construct
  source 1..74: mol_type = other RNA; note = dgRNA tracrRNA (option 1)
aacagcatagcaagttaaaataaggctagtccgttatcaacttgaaaaagtggcaccgagtcggtgcttttttt

SEQ ID NO: 147 - RNA, 22 nt, synthetic construct
  source 1..22: mol_type = other RNA; note = dgRNA crRNA backbone (option 2)
gtttaagagctatgctgttttg

SEQ ID NO: 148 - RNA, 74 nt, synthetic construct
  source 1..74: mol_type = other RNA; note = dgRNA tracrRNA (option 2)
aacagcatagcaagtttaaataaggctagtccgttatcaacttgaaaaagtggcaccgagtcggtgcttttttt

SEQ ID NO: 149 - skipped

SEQ ID NO: 150 - protein, 1368 aa, synthetic construct [sequence omitted]

SEQ ID NO: 151 - protein, 1393 aa, synthetic construct
  source 1..1393: note = SpCas9 Variant 1
MAPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVHHHHHH

SEQ ID NO: 152 - protein, 1393 aa, synthetic construct
  source 1..1393: note = SpCas9 Variant 2
MAPKKKRKVDKKYSIGLDIGTNSVGWAVITDEYKVPSKKFKVLGNTDRHSIKKNLIGALLFDSGETAEATRLKRTARRRYTRRKNRILYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIEEFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVHHHHHH

SEQ ID NO: 153 - protein, 1399 aa, synthetic construct
  source 1..1399: note = SpCas9 Variant 3
MGSSHHHHHHHHENLYFQGSMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKV

SEQ ID NO: 154 - protein, 1396 aa, synthetic construct
  source 1..1396: note = SpCas9 Variant 4
MAHHHHHHGGSPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKV

SEQ ID NO: 155 - protein, 1386 aa, synthetic construct
  source 1..1386: note = SpCas9 Variant 5
MAPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADHHHHHH

SEQ ID NO: 156 - protein, 1389 aa, synthetic construct
  source 1..1389: note = SpCas9 Variant 6
MAHHHHHHGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKV

SEQ ID NO: 157 - protein, 1399 aa, synthetic construct; identical to SEQ ID NO: 153
  source 1..1399: note = SpCas9 Variant 7

SEQ ID NO: 158 - protein, 1393 aa, synthetic construct
  source 1..1393: note = SpCas9 Variant 8
MAPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNAVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVHHHHHH

SEQ ID NO: 159 - protein, 1393 aa, synthetic construct
  source 1..1393: note = SpCas9 Variant 9
MAPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEALYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPALESEFVYGDYKVYDVRKMIAKSEQEIGKATAKYFFYSNIMNFFKTEITLANGEIRKAPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVHHHHHH

SEQ ID NO: 160 - protein, 1393 aa, synthetic construct
  source 1..1393: note = SpCas9 Variant 10
MAPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLADDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPALESEFVYGDYKVYDVRKMIAKSEQEIGKATAKYFFYSNIMNFFKTEITLANGEIRKAPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVHHHHHH

SEQ ID NO: 161 - protein, 1393 aa, synthetic construct
  source 1..1393: note = SpCas9 Variant 11
MAPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTAFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGALSRKLINGIRDKQSGKTILDFLKSDGFANRNFMALIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRAITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVHHHHHH

SEQ ID NO: 162-168 - skipped (7 entries)

SEQ ID NO: 169 - DNA, 80 bp, synthetic construct
  source 1..80: mol_type = other DNA; note = Human beta-globin (WT)-encoding ssODN sequence 9 (80 bp Forward)
agcaacctcaaacagacaccatggtgcatctgactcctgaggagaagtctgccgttactgccctgtggggcaaggtgaac

SEQ ID NO: 170 - DNA, 80 bp, synthetic construct
  source 1..80: mol_type = other DNA; note = Human beta-globin (WT)-encoding ssODN sequence 10 (80 bp Reverse)
gttcaccttgccccacagggcagtaacggcagacttctcctcaggagtcagatgcaccatggtgtctgtttgaggttgct

SEQ ID NO: 171 - DNA, 100 bp, synthetic construct
  source 1..100: mol_type = other DNA; note = Human beta-globin (WT)-encoding ssODN sequence 11 (100 bp Forward)
tgtgttcactagcaacctcaaacagacaccatggtgcatctgactcctgaggagaagtctgccgttactgccctgtggggcaaggtgaacgtggatgaag

SEQ ID NO: 172 - DNA, 100 bp, synthetic construct
  source 1..100: mol_type = other DNA; note = Human beta-globin (WT)-encoding ssODN sequence 12 (100 bp Reverse)
cttcatccacgttcaccttgccccacagggcagtaacggcagacttctcctcaggagtcagatgcaccatggtgtctgtttgaggttgctagtgaacaca

SEQ ID NO: 173 - DNA, 120 bp, synthetic construct
  source 1..120: mol_type = other DNA; note = Human beta-globin (WT)-encoding ssODN sequence 13 (120 bp Forward)
ctgacacaactgtgttcactagcaacctcaaacagacaccatggtgcatctgactcctgaggagaagtctgccgttactgccctgtggggcaaggtgaacgtggatgaagttggtggtga

SEQ ID NO: 174 - DNA, 120 bp, synthetic construct
  source 1..120: mol_type = other DNA; note = Human beta-globin (WT)-encoding ssODN sequence 14 (120 bp Reverse)
tcaccaccaacttcatccacgttcaccttgccccacagggcagtaacggcagacttctcctcaggagtcagatgcaccatggtgtctgtttgaggttgctagtgaacacagttgtgtcag

SEQ ID NO: 175 - DNA, 110 bp, synthetic construct
  source 1..110: mol_type = other DNA; note = Human beta-globin (WT)-encoding ssODN sequence 15 (with one missense; 111 bp Forward)
ctgacacaactgtgttcactagcaacctcaaacagacaccatggtgcatctgactcctgaggagaagtctgcggttactgccctgtggggcaaggtgaacgtggatgaag

SEQ ID NO: 176 - DNA, 110 bp, synthetic construct
  source 1..110: mol_type = other DNA; note = Human beta-globin (WT)-encoding ssODN sequence 16 (with one missense; 111 bp Reverse)
cttcatccacgttcaccttgccccacagggcagtaaccgcagacttctcctcaggagtcagatgcaccatggtgtctgtttgaggttgctagtgaacacagttgtgtcag

SEQ ID NO: 177-180 - skipped (4 entries)

SEQ ID NO: 181 - DNA, 80 bp, synthetic construct
  source 1..80: mol_type = other DNA; note = Human beta-globin (E6V)-encoding ssODN sequence 1 (80 bp F)
agcaacctcaaacagacaccatggtgcatctgactcctgtggagaagtctgccgttactgccctgtggggcaaggtgaac

SEQ ID NO: 182 - DNA, 80 bp, synthetic construct
  source 1..80: mol_type = other DNA; note = Human beta-globin (E6V)-encoding ssODN sequence 2 (80 bp R)
gttcaccttgccccacagggcagtaacggcagacttctccacaggagtcagatgcaccatggtgtctgtttgaggttgct

SEQ ID NO: 183 - DNA, 100 bp, synthetic construct
  source 1..100: mol_type = other DNA; note = Human beta-globin (E6V)-encoding ssODN sequence 3 (100 bp F)
tgtgttcactagcaacctcaaacagacaccatggtgcatctgactcctgtggagaagtctgccgttactgccctgtggggcaaggtgaacgtggatgaag

SEQ ID NO: 184 - DNA, 100 bp, synthetic construct
  source 1..100: mol_type = other DNA; note = Human beta-globin (E6V)-encoding ssODN sequence 4 (100 bp R)
cttcatccacgttcaccttgccccacagggcagtaacggcagacttctccacaggagtcagatgcaccatggtgtctgtttgaggttgctagtgaacaca